(19) United States
(12) Patent Application Publication
(10) Pub. No.: US 2026/0240166 A1
(43) Pub. Date: Aug. 20, 2026

(54) ANTIMICROBIAL AND ANTIVIRAL, BIOLOGICALLY ACTIVE POLYMER COMPOSITES EFFECTIVE AGAINST SARS-COV-2 AND OTHER VIRAL, BACTERIAL AND FUNGAL TARGETS, AND RELATED METHODS, MATERIALS, COATINGS AND DEVICES

(71) Applicants: David John VACHON, Spokne, WA (US); Mark Robert VACHON, Spokane, WA (US)

(72) Inventors: David John VACHON, Spokne, WA (US); Mark Robert VACHON, Spokane, WA (US)

(21) Appl. No.: 18/759,983

(22) Filed: Jun. 30, 2024

Related U.S. Application Data
(63) Continuation of application No. 18/129,854, filed on Apr. 1, 2023, now abandoned.
Application No. 18/129,854 is a continuation of application No. 17/775,304, filed on Nov. 11, 2020, now abandoned.
(60) Provisional application No. 62/934,510, filed on Nov. 12, 2019.

(51) Int. Cl. A01N 59/20; A01N 25/12; A01P 1/00; A61L 27/04; A61L 27/14; A61L 27/54

(52) U.S. Cl. CPC A01N 59/20; A01N 25/12; A01P 1/00; A61L 27/047; A61L 27/14; A61L 27/54; A61L 2300/408; A61L 2420/04

(57) ABSTRACT

Biologically active ion-exchange polymer salts are made by exchanging biologically active ionic agents onto organic ion exchange polymers. The activated polymers are uniquely surface active for use in a wide range of antibacterial, antifungal and antiviral coatings, materials, and devices, to minimize or prevent pathogen transmission and/or infection of a susceptible individual such as a patient in a hospital. The resultant coatings, materials, and devices are the foundation of a system enabling the construction of spheres of protection, for example to protect against transmission of COVID-19 (SARS-COV-2) coronaviruses and other viral and fungal pathogens in a clinical or institutional environment, for example hospitals, community institutions, cruise ships, and long-term care facilities. The activated ion-exchange polymer salts are process stable, allowing size reduction and formulation of the resulting powders into bulk polymers or polymer precursors to produce stable, biologically active polymer composites, including antimicrobial and antiviral polymer composites.

### TECHNICAL FIELD

The present disclosure relates to biologically active materials employing functionalized structurally organic ion-exchange materials associated with active antimicrobial agents, therapeutic agents and other biologically active agents, and solid polymer composites integrating these biologically active materials within novel compositions, coatings and devices that minimize and prevent pathogen-related disease.

### BACKGROUND

Increased human population and intermingling of populations have facilitated pathogen transmission and rendered it more difficult to control disease spread. Until 1987, the Centers for Disease Control and the American Hospital Association focused on patients as the principal vector for pathogen transmission, because the CDC regarded nosocomial infections to be generally unrelated to microbial contamination of (Cozad, A., and R. D. Jones. 2003) Evidence now clearly establishes that fomites (objects and surfaces that can become contaminated with pathogenic microorganisms) play a key role in spreading infection, including nosocomial infections. (Aitken, C., and D. J. Jeffries. 2001, Barker, J., D. Stevens, and S. F. Bloomfield. 2001)

Fomites readily serve as vehicles for transmission to living subjects (England, B. L. 1982; Haas, C. N., J. B. Rose, and C. P. Gerba. 1999; Reynolds, K. A., P. Watts, S. A. Boone, and C. P. Gerba. 2005; Sattar, S. A. 2001). Fomites readily become contaminated by direct contact with body secretions or fluids, soiled hands, aerosolized virus generated via talking, sneezing, coughing, or vomiting, or airborne virus settling after disturbance of a contaminated fomite. Once a fomite is contaminated, the transfer of contamination may readily occur between the contaminated fomite and another fomite or living host (Goldmann, D. A. 2000)

As rates of nosocomial (Health Care-Associated) and infections in hospitals, long-term care facilities, dental offices, veterinary care centers, daycare facilities, schools, gyms and other public places continue to increase, there is a growing urgency to develop materials that can minimize or prevent the survival of pathogens on surfaces located in these environments. It is important to recognize that many of these infections are resistant to antimicrobial drugs, hence these infections are referred to as antimicrobial resistant (AMR) or Multi-Drug Resistant (MDR).

A related need exists for materials that disable, inactivate, or kill contaminating pathogens following contact with the materials, to prevent transmission from a fomite surface composed of, or treated with, the materials to another fomite or a living subject.

Additional objects that remain unsatisfied in the art include development of contamination-resistant and contamination-preventive and infection-preventive materials that can be utilized in a variety of materials, devices and applications.

Further beyond the reach of current technology are polymer materials having a broad range of intrinsic surface biologically active properties, where the materials can incorporate a large diversity of surface active agents and can be incorporated in diverse compositions and methods and adapted for broad use in clinical, medical, personal, hygiene, environmental, and therapeutic methods and materials.

### SUMMARY OF EXEMPLARY EMBODIMENTS OF THE INVENTION

The invention fulfills these needs and satisfies additional objects and advantages by providing novel, insoluble organic and organometallic polymer materials that are biologically active by incorporating ionic biologically active agents, for example ionic antimicrobial agents (e.g., ionic or ionizable forms of antibiotic agents, antiseptic agents, antifungal agents, and antiviral agents, etc.).

The incorporation of/association of biologically active ionic agents with novel polymeric biomaterials and coatings of the invention is achieved by combining one or more ionic biologically active agents with an organic-based, ion-exchange polymer salt or its acid form, such as a functionalized organic-based ion-exchange resin material.

Organic/organometallic ion exchange resin materials are compounds comprising principally carbon and these materials are fundamentally distinct from inorganic ion exchange materials, such as mineral-based zeolites or nanoporous clay materials such as montmorillonite. Zeolites are known to impart decreased matrix compatibility with organic polymer materials likely resulting from weak Van de Waals interactions between the mineral structure and the polymer matrix into which it can be compounded and subsequently cast, molded or extruded. In exemplary embodiments, an organic or organometallic porous ion-exchange resin material is combined with a cationic or anionic biologically active agent (e.g., a cationic antibiotic, oligodynamic metal, or anionic anticoagulant) in an aqueous medium under conditions that mediate substitution of the ionic active agent onto the organic resin (by salt exchange or protonation)—typically by displacement of a similarly charged anionic or cationic counter-ion originally bound (ionically bound, electrostatically surface-associated, or adsorbed) onto the organic resin (or non-resin polymer) to form a substituted, biologically active polymer salt. Organic ion-exchange resin materials are typically insoluble, crosslinked polymers comprising functional groups that possess binding affinities for charged (cation or anion) compounds. Under the proper conditions the charged compounds can be bound to the resin in high, measurable concentrations defined herein as “exchange capacity” (EC).

In more detailed embodiments of the invention, a “biologically active” organic or organometallic ion-exchange polymer salt results by ionically modifying the organic resin to carry a biologically active ionic agent, for example, ionic silver (Ag⁺) substituted for a like-charged counter-ion originally bound onto the organic resin, for example ionic sodium (Na⁺) or the addition of Ag⁺ by the replacement of H⁺. The resulting activated ion-exchange polymer salt material is processed using novel materials and methods. In certain embodiments, the biologically active polymer salt particles are refined to uniform distributions of smaller sizes using a novel size reducing milling technology.

The biologically active polymer salts of the invention are useful, alone and in a diverse array of antimicrobial and other biologically active polymer “composite” materials. Within certain embodiments, a biologically active fine particulate ion-exchange polymer salt material is combined with thermoset, thermoplastic, or photocuring polymers, solvent soluble or miscible polymers, or with water soluble or water miscible polymers to yield castable, or thermally processable mixtures capable of forming solid, stable, activated polymer composite materials and surfaces.

In yet another detailed embodiment of the invention, a modified ion-exchange polymer salt is constructed by ionically modifying the polymer salt to carry an ionic agent, for example, ionic barium (Ba⁺⁺) substituted for a like-charged counter-ion originally bound on the organic resin, for example ionic sodium (Na⁺). These particulates can act as radiocontrast agents for X-ray imaging in methods and compositions currently employing barium sulfate, with less safety concerns (e.g., due to retention and/or soluble barium exposure). In illustrative embodiments radiocontrast effective ionic agents, exemplified by barium, are incorporated in an ion-exchange polymer salt, which can be used directly (although it will typically milled to a desired particle size) for gastrointestinal (GI) imaging, e.g., by delivering a suspension or colloid of the radiocontrast effective activated polymer salt to a GI tract of a patient (e.g., by ingestion) using conventional barium GI imaging tools and methods. In related embodiments, catheters, endoscopes, laparoscopic instruments and other devices are provided that integrate polymer composite materials made using radiocontrast effective activated polymer salts (including silver (Ag⁺) as described herein. In one exemplary embodiment, a portion (such as a longitudinal stripe) of an angioplasty tube, or ureteral stent is radiocontrast marked for localization within a vascular site by incorporating a radiocontrast effective activated polymer composite within a portion (e.g., a linear stripe portion) of the device (e.g., by co-extrusion with another polymer).

Milling methods are also provided herein employing a porous particulate ion-exchange polymer salt material activated by incorporation of an ionic biologically active agent. In exemplary embodiments, the activate porous polymer salt material is subjected to high energy bead milling employing a liquid non-solvent, such as water. This method is referred to as colloidal milling. Dry bead milling may also be employed to produce small uniform sized particle distributions, particularly for resins that, once synthesized, are harder than the starting resin. Examples include Cu(II), Zn(II), Fe(II), and Ti(II) salts of weak cation exchange resins and strong cation exchange resins (WCERs and SCERs). High energy jet milling or cryogenic jet milling may also be used.

Exemplary compositions of the invention are alternatively referred to herein in abbreviated form, by adding the incorporated ion at the beginning of the acronym followed by the resin type, e.g. Ag-SCER Silver strong cation exchange resin), Cu(II)-WCER (Copper (II) weak cation exchange resin), BA (benzalkonium etc.) such as BA-SCER. Other examples include Iron (II)-SCER, Fe(II)-SCER.

The methods and material compositions of the invention can employ or integrate a large diversity of antimicrobial agents. In additional embodiments, these methods and material compositions can incorporate numerous other biologically active ionic or ionizable agents (such as amines that can be protonated), including a diverse array of clinically useful and therapeutic agents.

In certain embodiments of the invention, fine particulate biologically active organic resin materials are incorporated into solid polymers to create solid polymer composites, and these materials provide an astounding array of useful constructs, textiles, objects, devices, coatings, laminates and the like for use in health care, institutional, personal, medical, environmental, laboratory, community and other settings. In exemplary applications, the materials and constructs of the invention are useful in medical, dental, orthopedic and veterinary facilities, hospitals, clinics, long-term care facilities, retirement homes, schools, daycare centers, universities, dormitories, hotels, community centers to include bus depots, train stations, stadiums, entertainment venues, and the like, tools, materials, implants, devices and equipment.

In other exemplary embodiments the biologically active organic resin materials are incorporated into solid polymers and the polymer compositions are pelletized to enable fabrication by molding, extrusion, and other processing methods.

Other uses and constructions of the materials and methods herein are described for consumer products, textiles, apparel, athletic equipment and accessories, sports therapy and gymnasium facilities and equipment, lavatory and food delivery, packaging, and transfer materials and equipment, transportation materials and equipment, and HVAC materials, to include filters, heat exchangers, ductwork, and equipment, water filters and tubing to remove microbial contaminants in water and aqueous (food) products, water storage tank coatings as for use for ships, submarines, and space exploration vehicles to prevent biofilm formation and PVC piping to prevent biofilm formation.

Within certain embodiments of the invention, methods for producing fine particulate organic ion-exchange polymer salt materials are described, allowing for biological activation of the polymer salt by ion exchange of the organic or organometallic starting material ion-exchange resin with a biologically active ionic agent. According to these methods generally, particles of a water-insoluble polysulfonated, polycarboxylated, polyaminated, or polyphosphorylated organic polymer salt material, for example a polymer salt of a cross-linked, functionalized organic resin, are combined with a biologically active ionic agent in an aqueous medium under conditions to facilitate exchange substitution of the biologically active ionic agent onto the organic ion-exchange resin polymer salt material by salt-exchange displacement of its counter-ion (e.g., a sodium ion or proton) initially associated with the organic resin. This yields a biologically active ion-exchange polymer salt in particle form having the biologically active ionic agent ionically associated with the organic ion-exchange polymer salt material. According to the teachings herein, the biologically active ionic agent is thus rendered insoluble and will not freely dissociate from the biologically active polymer salt material in a deionized aqueous medium. A wide array of biologically useful ionic or ionizable drugs, compounds and other active agents can be employed to form the biologically active ion-exchange polymer salts of the invention. In exemplary embodiments, the biologically active ionic agent is an antimicrobial agent. Suitable antimicrobial agents include ionic or ionizable antibiotics, antiseptics, antivirals, antiparasitic compounds, and antifungal compounds, oligodynamic metals, and ions capable of secondary reactions to form reactive oxygen species (ROS). In other exemplary embodiments, an oligodynamic metal selected from silver, copper, zinc, iron, gallium, or bismuth is employed. In other exemplary embodiments, a cationic antibiotic is employed. Exemplary cationic antibiotics include tetracyclines or anthracycline and aminoglycosides. In more detailed embodiments, a tetracycline is selected from tetracycline, doxycycline, minocycline, oxycycline, lymecycline, or apicycline, or combinations thereof and aminoglycosides include gentamicin and/or tobramycin. In yet additional exemplary embodiments, a cationic antiseptic is employed. Exemplary cationic antiseptics may comprise a guanidinium group (e.g., as exemplified by chlorhexidine or polyhexamethylenebiguanide), or a quaternary ammonium group (e.g., as exemplified by benzalkonium, cetylpyridinium, cetrimonium (cetrimide) and other quaternary ammonium compounds).

In more detailed embodiments, polycationic polymers (or their polyamine forms) such as polylysine or chitosan are bound to the organic ion-exchange polymer to generate biologically active ion exchange salts/complexes that can be used in wound dressings that can facilitate coagulation in cases of hemorrhage. In such instances, the polycationic polymer-ion exchange polymer complex can be incorporated into a foam that can be forcibly delivered to a site of injury to stop the bleeding. In other embodiments, water soluble ion exchange polymers such as polystyrene sulfonate in salt or acid form can be combined with the polycationic polymers (or their polyamine forms) to form complexes that can be formulated into gels that can be delivered to the site of hemorrhage using a syringe.

In additional exemplary embodiments, anionic biologically active agents are incorporated by ionic association within the organic ion-exchange polymer salts of the invention. Exemplary biologically active anionic agents (including agents modifiable to an anionic form) include acetylsalicylic acid-CO₂—, dexamethasone sodium phosphate, fusidic acid (fusidate), and vitamin C (ascorbate), among others. 2 Exemplary ion-exchange polymer salts for use within the invention may comprise an ion-exchange polymer salt comprising one or more of a styrene, acrylic, acrylate, sulfonate, carboxylate, phosphate, protonated amine (ammonium), and/or quaternary ammonium functional group(s). In certain embodiments, the ion-exchange polymer salt material comprises a cross-linked organic polymer resin, for example a cross-linked styrene, acrylic, or acrylate polymer resin.

In more detailed aspects of the invention, novel biologically active polymer “composites” with enhanced mechanical stability (as compared to zeolite compositions) and methods for preparing these composites, are provided. In exemplary embodiments, the biologically active composites are made by first providing an ion-exchange polymer salt, as summarized above. The organic ion-exchange polymer salt is typically a water-insoluble polysulfonated, polycarboxylated, polyaminated, or polyphosphorylated polymer salt. In exemplary embodiments the particles have a porous construction, with individual particles defining channel, void and pore space surrounded by walls and partitions of polymer salt material. The ion-exchange polymer salt particles are generally admixed with a biologically active ionic or ionizable agent in an aqueous medium to substitute the biologically active ionic agent by salt-exchange for a counter-ion initially associated with the ion-exchange polymer salt material or for protonation by the ion exchange material. This yields a biologically active porous ion-exchange polymer salt particle having the biologically active ionic agent ionically associated with the ion-exchange polymer salt material. By virtue of this novel preparation method and construction, the biologically active ionic agent is rendered insoluble, preventing free dissociation from the insoluble ion-exchange polymer salt material in deionized water.

Following synthesis of the active ion-exchange polymer salt material, the material is dried to remove most, or all of the water present (e.g., water or an aqueous solution such as including an alcohol). The biologically active ion-exchange polymer salt particles are then milled by a high energy milling process. This may involve the use of dry or colloidal milling with a non-solvent liquid such as water to provide optimal particle size distributions appropriate for generating defect free composites.

The resultant fine particulate biologically active ion-exchange polymer salt material is optionally blended with thermoset or thermoplastic or photocuring polymer precursors to form a fluid or semi-solid thermoset or thermoplastic or photocuring polymer composite mixture. This mixture comprises the fine particles of biologically active ion-exchange polymer salt thoroughly or incompletely admixed with the polymer precursors (e.g., to form homogeneous or heterogeneous dispersions, or to blend the polymer salt particles only through a discrete portion of the composite mixture). After blending to a desired degree of mixing, thermoset or thermoplastic or photocuring polymer precursors are hardened or cured to form the biologically active solid polymer composite.

Any biologically acceptable thermoset or thermoplastic polymer can be employed within these aspects of the invention or in the case of aspects not requiring biologically acceptable polymers, industrial grade materials are acceptable. In exemplary embodiments, the thermoset or thermoplastic or photocuring polymer is selected from the group consisting of polysiloxane, polyalkylene, polyamide, epoxy, polycarbonate, polyester, vinyl, acrylic, polyurethane, polymers and combinations thereof. In certain embodiments, the polymer precursors comprise non-vulcanized (uncured) silicone rubber precursors.

Free radical and acetoxy silicone rubber precursors can be combined so as to form silicone polymer composites that can be cured under a range of conditions, for example at about 150 degrees for 5 to 10 minutes or with the addition of exposure to UV radiation or at room temperature as with acetoxy-curing formulations. Within more detailed embodiments, the biologically active ionic agent is an oligodynamic metal and the activated fine particulate product incorporating the oligodynamic metal is blended with silicone gel, high consistency rubber (HCR), or liquid silicone rubber (LSR) further comprising an oligodynamic metal modified ion exchange resin.

Also provided within the invention are materials and composites made according to the foregoing processes, and articles and devices incorporating these materials and composites. In certain aspects, biomaterials, products, tools and equipment are made that incorporate a fine particulate, biologically active ion-exchange polymer salt or resin material as described. Biologically active polymer composites of the invention remain intact and biologically active without substantial chemical degradation, oxidation, hydrolysis, chemical decomposition, or photodegradation of the integrated ionic biologically active agent (e.g., wherein the biologically active agent remains stable and retains most if not all of its biological activity during preparation of the ion-exchange polymer salt, and preparation and hardening/curing of the thermoset or thermoplastic or photocuring polymer).

Additional novel aspects of the invention include the provision of novel materials and methods for producing “self-regenerating” or “renewable” biomaterials and polymer composites. Polymer composites described herein can passively renew or regenerate their original surface biological activity, or can be rehabilitated, restored or recharged to approximate their initial (post-fabrication) biological activity, after being partially or completely chemically exhausted, reacted, degraded, decomposed or discharged. In exemplary embodiments, a fine particulate biologically active ion-exchange resin material is integrated throughout a solid polymer structure to provide for passively renewable surface activation following discharge. For example, after a period of normal surface wear or erosion, polymer composites of the invention will often exhibit some of “discharge” of the biologically active ionic agents, including by release or dissociation of activated ion-exchange resin material and/or biologically active ionic agents from the polymer surface, chemical reaction, decomposition, photodegradation, at the polymer surface, and or loss by erosion of the fine particulate organic ion exchange polymer salt material embedded within the composite (or composite surface layer(s)). In these embodiments, “recharging” of biological activity at the polymer surface is passively mediated by erosive wear, which passively debrides an outermost layer of the composite and exposes underlying material that is fully active as intact, non-discharged particles of the activated organic ion exchange polymer salt—effectively restoring or replacing the original surface activity.

In related embodiments, erosive recharging is actively mediated, for example by debriding or polishing a partially discharged polymer composite surface with an abrasive paper, cloth, paste or solution. This manual resurfacing/recharging is likewise mediated by debriding a discharged surface layer of the polymer composite to expose fresh, non-discharged particles of the activated organic ion exchange polymer salt bearing a full (comparable to original, e.g., at least 75%-90% of original surface activity) load of surface active biologically active ionic agent. In alternative “recharging” constructions and methods, after partial discharge of biological activity from a polymer surface, the surface can be actively recharged by manual methods involving novel chemical re-treatment. For example, discharge of biologically active ionic agent comprising ionic copper (Cu⁺⁺—) from an activated polymer composite can occur when tissues or physiological fluids are contacted with the surface of the activated composite (e.g., by counter-ionic exchange of sodium (Na⁺) in the physiological fluid with silver ions originally “loaded” within the composite. This discharges some of the total copper ion activity (e.g., expressed in terms of antimicrobial activity) from an original “loading capacity” “selected sub-capacity loading” or “post-fabrication biological activity potential”. Under these circumstances, the invention provides novel materials and methods allowing for recharging or reloading (even above original loading or post-fabrication activity) of most or all of the original loading or activity potential, for example by treating a partially discharged composite surface with a solution of a copper salt (e.g., copper chloride). In other embodiments ionic antiseptics (e.g., benzalkonium-based antiseptics) can be similarly recharged as reloaded or simply under standard methods of disinfection with quaternary ammonium disinfectants while said surface provides complimentary and synergistic disinfection (e.g., by wiping or saturating the surface with quaternary salt (disinfecting) solution products such as a benzalkonium chloride solution).

Additional novel materials and methods are provided where a polymer composite surface containing a biologically active ionic agent (e.g. copper (II) or iron (II)) can be “surface activated”. In one exemplary embodiment, a composite surface is activated by exposing the surface to hydrogen peroxide, H₂O₂ (e.g., simply by wiping, immersing or spraying the surface with a peroxide solution of ≤8%, or in the process of disinfection using H₂O₂ vapor (VHP) which is generally of 30-35% concentration. This generates reactive oxygen species (ROS) at the surface of the material by the ion exchange release of small amounts of Cu(II) or Fe(II) in the presence of hydrogen peroxide to yield the active ROS hydroxyl radical, rendering the surface more strongly antimicrobial.

The forgoing and additional objects, features, aspects and advantages of the present invention will become apparent from the following detailed description.

### BRIEF DESCRIPTION OF THE DRAWINGS

Embodiments of the disclosure are described below with reference to the following accompanying drawings.

FIG. 1 is a thermogravimetric analysis (TGA) profile of IRP69-Ag heated from 100 to 400° C. heated at 50° C./minute. As evident from this TGA profile, less than 2% weight loss is observed up to 400° C. and just a fraction of 1% up to 300° C., the approximate processing temperature for polyethylene terephthalate for example.

FIG. 2 is a graph depicting antibacterial activity results for silicones incorporating fine particulate activated polymer salt particles (IRP69) comprising biologically active counter-ions of Ag, Cu, Ag/Cu, and benzalkonium evaluated over a three week period during which time the samples were extracted in 0.9% normal saline at 37° C. during the time course of the study.

### SCER-Ag Produced by Dry Milling.

FIG. 3 is a graph detailing the results (reduction in bacterial counts) of a modified ASTM E2180 assay (ASTM International, West Conshohocken, PA, 2007) utilizing Staphylococcus aureus against modified Q7-4750 silicone rubber modified to include Sulfonated polystyrene-co-divinylbenzene (IRP69) including varying concentrations by weight of IRP69-Ag (0.25-5.0 wt. %) following extraction in PBS at 37° C. The modification of the method involves using a small-pore mesh made out of polypropylene to evenly distribute an agar slurry (0.01 M PBS, 0.0033 w/v % agar) inoculated with 10⁵ Staphylococcus aureus onto the surface at intervals before and after extraction. The assay reveals that after four weeks of extraction in 0.01 M PBS the silicones modified with 1.0, 2.0, and 5.0% IRP69-Ag yielded reductions of 6-logs against the bacterium.

FIG. 4 is a graph demonstrating the percent reduction results of a modified ASTM E2180 assay (ASTM International, West Conshohocken, PA, 2007) using an inoculum of 10⁶ E. coli against modified Q7-4750 silicone rubber modified to include Sulfonated polystyrene-co-divinylbenzene modified Ag (2.0 wt % Ag-SCER), benzalkonium (QAC) (2.0 wt % BA-SCER), and a binary (Ag/QAC) at 2.0 wt % (1.0 wt. % of each Ag-SCER and BA-SCER) all compared to an non-modified control silicone (Q7-4750). These data reveal that the Ag and Ag/QAC-modified were capable of killing 100% of the inoculum. The QAC alone demonstrated an approximately 60% reduction in the bacterial counts, surprisingly given that QAC is known for effectiveness against gram positive species.

FIG. 5 is a graph demonstrating the percent reduction results of a modified ASTM E2180 (ASTM International, West Conshohocken, PA, 2007) assay using an inoculum of 10⁶ E. coli against Sulfonated polystyrene-co-divinylbenzene modified Q7-4750 silicone rubber to include Ag (2.0 wt. %) compared to an non-modified control silicone (Q7-4750). These data reveal that the IRP69-Ag-modified silicones were capable of killing essentially 100% of the inoculum even with exposure to 10% fetal bovine serum (FBS) in PBS indicating that protein adsorption had no significant effect compared to the control silicone rubber.

FIG. 6 is a graph comparing the thermal stability of the benzalkonium salt of IRP69 (IRP69-benzalkonium) to the stability of benzalkonium chloride.

FIG. 7 is a graphical representation of a time-to-kill assay following a 10⁸ CFU inoculation of E. coli onto a silver-modified, strong cation exchange resin (Ag-SCER)-modified silicone composite as measured by ASTM E2180. Following a 3-hour adhesion step the samples are removed and lightly rinsed and placed into media. At designated time points, the bacteria are removed from the surface and counted. Within 24-hours all bacteria have been killed representing a >7 log 10 reduction.

FIG. 8 is a graphical representation of a time-to-kill assay for Escherichia coli 67 in a flow system. In this experiment, 300 cc of artificial urine (Brooks T, Keevil CW. A simple artificial urine for the growth of urinary pathogens. Lett Appl Microbiol. 1997 March; 24 (3): 203-6. doi: 10.1046/j.1472-765x.1997.00378.x. PMID: 9080700) are circulated through a 24-inch segment of Ag-SCER-modified (1.5 wt %) silicone tubing (7 Fr O.D.) for 2-hours at 37° C. and 10e5 CFUs of Escherichia coli 67 added. Within 4-hours all bacteria had been killed (FIG. 8), demonstrating surprising and unprecedented antimicrobial efficacy.

FIG. 9 is a graphical representation of a time-to-kill assay for Escherichia coli 67 in a flow system. In this experiment, 300 cc of artificial urine are circulated through a 24-inch segment of Ag-SCER-modified (1.5 wt %) silicone tubing (7 Fr O.D.) for 2-hours at 37° C. and 10e7 CFUs of Escherichia coli 67 added. Somewhere between 8-24-hours all bacteria had been killed. The killing of this organism at this high inoculum is an unprecedented and surprising finding. 1.5 wt % Ag-SCER composite can kill E. coli 67 in a flow system. 1×10⁷ CFUs E. coli 67 in 300 mL of artificial urine was pumped through either test or control material for up to 48 hours. The test material kills E. coli at a point between 8 and 24 hours post inoculation. Very likely, the remaining 10e3-10e4 organisms are killed off within a period of 4-hours or less as demonstrated by FIG. 9.

FIG. 10 is a graphical comparison of the effectiveness of a silicone composite comprising 1.5 wt % of Ag-SCER in different urine formulations, (AU, AUK and Pooled Human Urine).

FIG. 11 is a graphical representation of the cellular toxicity of a silicone composite comprising 1.5 wt % of Ag-SCER against HEK293 (kidney) and T24 (bladder) cells in culture.

FIG. 12 is a depiction of a catheter hub incorporating a biologically active ion-exchange polymer salt particle comprising Ag⁺ or Cu(II). When the housing of the catheter hub comprises Cu(II)-modified biologically active ion-exchange polymer salt particles to include any septum at the interface of the hub and the catheter lumen, the surface can be activated by saline to liberate Cu(II) in the form of CuCl₂ and when the catheter hub comprises Ag-modified biologically active ion-exchange polymer salt particles, the surface can be activated by saline to liberate Ag⁺ in the form of AgCl. When the housing of the catheter hub comprises Cu(II)-modified biologically active ion-exchange polymer salt particles, the surface can be activated by saline and H₂O₂ collectively to produce hydroxyl radical. The ions are released in very small quantities by ion exchange with NaCl by contact activation of the catheter hub polymer surface with a cap (comprising a foam insert) that incorporates a solution of saline or a solution of saline with/and H₂O₂. Hydroxyl radical can also be produced from a surface modified with Fe(II)-modified biologically active ion-exchange polymer salt particles.

FIG. 13 is a depiction of a battery separator fabricated using Li-SCER and/or Li-WCER placed between the anode and cathode of a lithium ion battery.

FIG. 14 is a depiction of a fenestrated silicone wound contact layer.

FIG. 15 is a diagrammatic representation of a patient clinical environment for use of antiviral and antifungal composites and coatings of the invention, to reduce clinical transmission of fungi and viruses.

### DETAILED DESCRIPTION OF EXEMPLARY EMBODIMENTS OF THE INVENTION

Described herein are compositions of polymeric ion-exchange materials incorporating biologically active ionic agents, i.e. biologically active ion-exchange polymer salts. These materials are useful for a variety of purposes, including as stable biologically active constituents of uniquely functional solid polymer composites. The activated or derivatized ion-exchange polymer salts can be combined with thermoplastic or thermoset polymer precursors to generate biologically active polymer composite mixtures, including moldable, extrudable, layerable and paintable, activated polymer composite mixtures. These mixtures can be hardened or cured to make uniquely surface activated hardened polymer composite materials, coatings, fibers, textiles, devices, furnishings and apparatus, among other products. Primary compositions of the invention comprise activated ion-exchange materials typically provided in the form of polymer salts, including activated salts of polymer resins (insoluble cross-linked polymers). Suitable ion-exchange polymers include cation-exchange polymers as well as anion-exchange polymers. The polymer salts incorporate one or more biologically active ionic agents, for example an ionic or ionizable antimicrobial agent (for example an ionic antimicrobial such as an oligodynamic metal, or ionic antibiotic, or an antimicrobial converted to an ionic form by chemical modification, such as by protonation.

A wide assemblage of ionic or ionizable antimicrobial agents can be incorporated in activated polymer salts of the invention, including antibacterial drugs, antibiotics, antiviral agents, antifungal agents, organometallic compounds, and oligodynamic metals. Other useful biologically active agents within the methods and compositions of the invention include antiseptics, antimycotics, anti-inflammatory agents, antiproliferative agents, antineoplastic agents, chemotherapeutic agents, antihypertensive agents, anti-arrhythmic agents, anticoagulants, antioxidants, antiparasitic agents, anticonvulsant agents, antimalarial agents, amine-containing pharmaceutical agents, and other therapeutic agents obtainable in ionic form for use within the compositions and methods of the invention.

Biologically active ionic or ionizable agents are captured or bound in an insoluble matrix by ionic association with ion-exchange polymers, often cation or anion-exchange polymer “resins.” Useful ion-exchange polymers are often insoluble in non-ionic aqueous media (e.g., distilled water and alcohols). In some embodiments the ion-exchange polymer may be insoluble or poorly soluble in non-ionic and ionic aqueous media. The subject ion-exchange polymers often possess some hydrophobic character, e.g., as is true for polymers comprising largely hydrocarbon monomers (such as divinylbenzene), where both ionic and non-ionic aqueous media will not substantially wet or saturate the ion-exchange polymer (typically wetting or hydration potential will be marked by less than 25% water saturability by weight of the material soaked in aqueous media, often less than 20%, less than 10%, or less than 5% w/w). Hydrophobicity and hydrophilicity are, in part, related to the relative amount of crosslinking of the organic ion exchange system and as such can be adjusted for different materials and uses according to the teachings herein (e.g., by altering the ionic component of the system, crosslink density, and/or counter-ion bound to the resin system).

Useful cation-exchange materials for constructing biologically active polymer salts may include weak or strong cation-exchange materials. Weak cation-exchangers may contain, for example, carboxyl (—CO₂⁻) functionalities (alternatively “moieties,” or terminal or side functional groups to include iminoacetic acid groups such as with Chelex 100). Strong cation-exchangers are exemplified by sulfonates (—SO₃⁻). In general, carboxylates have lower binding constants than do their strong cation-exchanging counterparts, such as sulfonates however the stability of complexes of di- and trications can be greatly enhanced by the chelation effect. Accordingly, carboxylates will give up (exchange, release or allow dissociation of) dications such as copper (II) and monocations such as silver (I) more readily than more electronegative functionalities (e.g., sulfonates).

In illustrative embodiments of the invention, strong cation-exchange materials are constructed comprising polysulfonated salts of polymerized styrene (polystyrene). In other illustrative embodiments, polyphosphorylated materials such
as cellulose phosphate or phosphates of synthetic organic structures are constructed. These polymeric ion-exchange materials, such as those based upon polystyrene, may be cross linked with divinylbenzene to form insoluble styrene-divinylbenzene copolymer materials with some hydrophilicity (water loving character) imparted by the binding of moieties with some dependence upon the amount of cross linking agent included. These materials can be crosslinked to form insoluble ion-exchange materials. Exemplary cross-linking agents include, but are not limited to, diacrylates to form acrylic-co-diacrylate copolymers or divinyl compounds such as divinylbenzene to form acrylic-co-divinylbenzene copolymers. Weak cation-exchange materials are also provided, exemplified by polycarboxylic acid materials (salts or protonated form) that may be acrylic structures formed by polymerization of acrylate materials. In alternative embodiments, cation-exchange materials can include any of a diverse selection of polymers, including styrene, acrylic, vinyl, sulfonate, carboxylate, and phosphate, among others. A variety of initial counter cations can be associated with the ion-exchange polymer base or scaffold, including for example sodium ions, potassium ions, and hydrogen ions.

Thus, in different exemplary embodiments of the invention cation-exchange resins are primarily functionalized as polysulfonated salts, polycarboxylated or salts, polyphosphorylated salts. In some embodiments, the ion-exchange polymer will include two or more different polymer salts. Exemplary ion-exchange polymer mixtures include blends of polysulfonates, polycarboxylates, or polyphosphonates. These can be biologically active by salt exchange according to the methods herein with any of a diverse selection of cationic biologically active agents, for example oligodynamic metal cations, organic cations, or mixtures of organic cations and metal cations.

Anion-exchange materials can include strongly basic or weakly basic anion-exchange materials. Strongly basic anion-exchange materials generally include poly(quaternary ammonium ion) salts and weakly basic anion-exchange materials generally include polyamines that are generally secondary amine structures but can include tertiary amines as well. These ion-exchange materials can be copolymers of styrene and divinylbenzene, sometimes referred to as styrene-divinylbenzene copolymers. In some embodiments, anion-exchange materials can include polymers such as styrene, vinyl, amine, quaternary ammonium as well as counter anions such as chloride ion, hydroxide ion or carboxylate ion, for example.

The anion- or cation-exchange materials may be functionalized as described and ionically bound to one or more biologically active ionic agents that possess a distinct biological activity (which may comprise a specific therapeutic efficacy, such as an antimicrobial or anti-inflammatory activity). Useful biologically active ionic agents include any biologically active agent (e.g., an antimicrobial or anti-inflammatory agent) that can be prepared in an ionic form, such as an ionizable salt form. The biologically active agent is loaded onto the ion-exchange polymer typically as a substitute counter-ion by ion-exchange to replace an initial counter-ion (e.g., Na⁺) and form a new, biologically active polymer salt. The biologically active replacement counter-ion can include any of a diverse selection of ionic or ionizable agents having a desired biological or therapeutic activity, including for example one or more of a metal cation, a quaternary ammonium compound, an organic ion, a protonated amine, a carboxylate, a phosphate, a cationic or anionic surfactant, and/or a biguanide. In exemplary embodiments, the counter-ion material can include one or more mono, di, and/or trivalent cation(s). Exemplary metal cations include, but are not limited to, Na⁺, Ag⁺, Au⁺, Cu⁺⁺, Ga+⁺⁺, Zn⁺⁺, and Ce⁺⁺⁺ Fe⁺⁺, and/or combinations thereof. Exemplary quaternary ammoniums include, but are not limited to, benzalkonium chloride, cetrimonium (cetrimide) chloride, and cetylpyridinium chloride. Exemplary protonated amines include, but are not limited to doxycycline hydrochloride, minocycline hydrochloride. Exemplary biguanides include, but are not limited to chlorhexidine diacetate, metformin, proguanil, and chlorproguanil. Useful biologically active cationic and anionic agents for binding to ion-exchange polymer materials include, but are not limited to, antimicrobial compounds including oligodynamic metal ions, charged pharmaceutical agents including therapeutic agents or drugs effective in the treatment and care of multicellular organisms, and other ionic substances that can improve a particular clinical or biological environment. Among exemplary antimicrobial agents illustrated here are antibacterial drugs, including antibiotics, antiviral agents, anticoagulants, antifungal agents, organometallic compounds, antiparasitic drugs, as well as oligodynamic metals. Exemplary therapeutic agents include, but are not limited to, anti-inflammatory agents, chemotherapeutic agents, antibiotics, antioxidants, antimalarials, contraceptive agents including spermicidal agents, amine-containing pharmaceutical agents and the like.

Useful ion-exchange polymer materials for association with biologically active ionic agents may be soluble or insoluble. In some embodiments, the ion-exchange polymer material is an insoluble matrix or support polymer, which can take the form of small particles or beads on the order of millimeters in diameter. Exemplary organic ion exchange resin materials of this type desirably possess porous particulate structures, with pores on the surfaces and channels and voids communicating with the surfaces of the resin particles. This porous construction enhances ion-exchange functionality of the resin particles (i.e., it increases ability of the particles to communicate with and exchange biologically active ions for original counter-ions associated with the resin material from which the particle is formed).

Exemplary organic ion-exchange polymers for use within the invention include monomers of one or more of styrene, acrylic acid, vinyl acetate, methacrylic acid, divinyl benzene, and/or butadiene, among others. In certain embodiments, the ion-exchange polymer is cross-linked to modify solubility and ion-exchange potential. Exemplary cross-linked polymers can incorporate units (segments) of, but are not limited to, polyarylenevinylsulfonate, polystyrene-sulfonate, polyvinylsulfonate, polyalkylenesulfonate polyantholesulfonate, and/or acrylamidomethyl propane sulfonate polymer. In one exemplary embodiment, a polystyrene is employed that is variably or adjustably crosslinked through addition of 0.1-55 mole % of divinylbenzene during polymerization—to create a range of selectable strength ion-exchange capacity (EC), loading potential (i.e., selectable total load capacity of biologically active counter-ion) and optionally a variable potential for dissociating the biologically active ion for drug delivery purposes (e.g., when in contact with physiological, ionic fluids and tissues).

Ion-exchange polymer materials for use within the invention are generally functionalized to bind to, or associate ionically with, cations or anions. For example, acrylics, styrenes and polyalkylenes may be functionalized by incorporating (binding to) one or more sulfonate, carboxylate and/or phosphorylate moieties (ions)—to form such exemplary useful polymers comprising arylenevinyl sulfonate, styrene sulfonate, vinyl sulfonate, methacrylic acid, acrylic acid, vinylphosphate, sulfonated divinylbenzene, or divinyl benzene. These polymers will typically be employed in a first (unactivated) polymer salt form, lacking the “biologically active ionic agent”, and instead having an inactive, “initial counter-ion” present to exchange with the biologically active agent, such as sodium (Na+).

Functionalized ion-exchange materials are often provided in the form of a “ion-exchange polymer salt”, for example sodium polystyrene sulfonate. Illustrating general “salt exchange” designs contemplated here, the Na+ ionic component of the first polymer salt (sodium polystyrene sulfonate (a polymetallosulfonate)), can be exchanged with any of a variety of biologically active (e.g., antimicrobial or therapeutically effective) metal cations to prepare mono, di, tri, and even tetravalent metal ion, “biologically active polymer salt” derivatives. Similarly, polymetallosulfonates such as sodium polystyrene sulfonate can be converted to a polyorganosulfonate derivative (e.g., by exchange of sodium for any nitrogen atom containing salt/protonatable nitrogen compound of interest). Exemplary nitrogen atom containing salts/protonatable nitrogen compounds (ionizable) for use in these aspects of the invention include amines, ammonium ions, amidines, amidinium ions, imines (iminium ions), thiazoles, imidazoles, guanidines, guanidinium ions, and/or pyridines, and pyridinium ions. In other illustrative embodiments, ammonium ion-exchange polymer salt derivatives can be produced by exposing amino compounds to acid forms of polymers, for example and acid form of polysulfonate. In yet additional illustrative embodiments, metal dication derivatives can be produced by exposing acid or salt forms to divalent metal salts. For example, the addition of Cu(II) to the sodium form of poly(co-methacrylic acid-divinyl benzene) to yield the Cu(II) salt of poly(co-methacrylic acid-divinyl benzene) has been shown to be effective for the purification of bacteria from tap water. Normally present at concentrations as high as 500 CFUs/mL, the bacterial counts of tap water can be reduced well below these concentrations by simply passing the stream across a bed of the resin. Applications for the purification of water include dental water for irrigation use during dental procedures. Commonly, dental unit water lines are susceptible to the formation of biofilms on the lumens of the supply tubing because of the extended dwell times that tubing can hold stagnant water. Because of this, the lines are routinely flushed with special antimicrobial flushing solutions to include bleach, H₂O₂, and silver citrate (Citrisil) for example.

To produce primary biomaterials of the invention comprising activated ion-exchange polymer salts, ion-exchange polymers are associated with biologically active counter-ions as shown in Reaction scheme 1. In this scheme an exemplary polysulfonated material is used, where Cat^{m+} is an organic or oligodynamic metal cation, R is a carbon containing group, m=z(q), where z and q are whole numbers greater than 1, n is a number greater than 1, and X is a counter-ion.

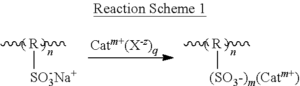

The (R)n is any oligomeric or polymeric backbone. The R group may include monomers such as arylenevinyl sulfonate, styrene sulfonate, divinyl benzene, and/or vinyl sulfonate monomers as well as nonsulfonated monomers. In some embodiments, the oligomer or polymer can include repeating units of the same monomer or a plurality of different monomers. The oligomer may be copolymerized with monomers and/or other oligomers to form a co-polymer. For example, the polymer backbone of polysulfonated cetylpyridinium salt may be polyarylenevinylsulfonate, polystyrene-sulfonate, polyvinylsulfonate, polyantholesulfonate, and/or acrylamidomethyl propane sulfonate polymer. In other embodiments a co-monomer may serve to crosslink the polymer to increase stability and decrease solubility or hydrophilic character.

In exemplary constructions of activated ion-exchange polymer salts, the initial ion-exchange polymer may be selected from a commercially available polymer, for example a commercially supplied polysulfonated resin, such as Amberlite™ IRP69 (Sodium Polystyrene Sulfonate USP, an insoluble, strongly acidic, sodium form cation-exchange resin supplied as a dry fine powder) or Amberlite™ IRP88 (Polacrillin Potassium NF, a weakly acidic potassium form cation-exchange resin supplied as a dry fine powder).

Insoluble ion-exchange materials can be created by cross-linking. At lower levels of cross linking (produced with a lower concentration of cross linking agent), the polymer may possess some hydrogel-like character, whereas at higher crosslink densities the absorption of water is minimized and solubility of the resin material is reduced to a point generally recognized in the art as “insoluble”. In exemplary embodiments, insoluble polysulfonated ion-exchange materials are created by addition/copolymerization of a vinyl derivative, such as styrene, or other monomers with a di- or tri-functional cross-linking agent such as divinyl benzene. In this and similar examples, the ion-exchange material, will typically have a crosslinking unit density or concentration in a range of between 0.1 and 20 mole percent, which will generally be correlated with a desired ion-exchange capacity (EC) of the resin. Desired ion-exchange capacities found useful for production of activated ion-exchange polymer salts of the invention will typically range between 0.1 and 20.0 mEq/gram.

In certain aspects of the invention, ion-exchange polymer salts are provided in particulate form before activation by salt exchange (i.e., to exchange the initial, inactive counter-ion with a biologically active ionic agent to form the activated ion-exchange polymer salt). Suitable particles sizes of ion-exchange polymers for preparation of activated polymer salts by salt exchange (to form biologically active ion-exchange polymer salt particles) will often have an average particle size or diameter in a range of a conventional organic ion exchange resin, for example from about 0.00025 mm to about 2.5 mm, about 0.0005 mm to about 1.5 mm, or about 0.075 mm to about 0.5 mm. In other embodiments, the particle size diameter of the ion-exchange polymer starting material will be from about 300-500 μm, or about 500-700 μm.

Exemplary ion-exchange polymer salts employ a polymer matrix that is effectively water insoluble. Insolubility as used here means that essentially all (at least 95-98%) of the subject polymer material remains insoluble in deionized water at room temperature. Generally, the polymer matrix will remain insoluble even in non-ionic or ionic solutions, such as tap water, saline, physiological fluids, or deionized water. In illustrative embodiments, sodium polystyrene sulfonate and poly(vinyl carboxylic acid), otherwise known as polyacrylic acid, sodium salts are water soluble materials. These and like materials can be rendered more or less insoluble for use within different aspects of the invention by variable cross-linking, as described. One exemplary useful strong cation exchange commercial product Amberlite IRP69, (Rohm and Haas Company, a subsidiary of Dow Chemical Company, Philadelphia, PA 19106-2399), is a sulfonated poly(styrene co-divinyl benzene) (crosslinked) ion-exchange resin. Another exemplary weak cation exchange commercial product for use within the invention is Amberlite IRP64, (Rohm and Haas Co.) a polymethacrylic acid, co-divinylbenzene (crosslinked) ion-exchange material. Both of these materials are essentially insoluble in water (by virtue of the divinylbenzene crosslinking of the polymers). Other varieties/brands of strong and weak cation exchange resins with differing levels of cross-linking and exchange capacity (EC) can be employed. Generally, the percentage of crosslinking agent is represented as mol %, however it may also be presented as wt % and by potential for swelling by water absorption. Ion-exchange capacity, hydrophobicity and insolubility are all generally directly proportional to amount or percentage of cross linking. By using greater or lesser percentages of cross linking, the ion-exchange capacity of polymers/resins within the invention can be varied, as can the potential for water absorption and for “reversible association” of loaded biologically active counter-ions (i.e., the potential to release the counter-ion from the activated (loaded) polymer salt into an aqueous medium or ionic fluid or tissue compartment by ionic dissociation). Cross-linked ion-exchange polymer salts are thermally stable, allowing for drying under vacuum at elevated temperatures (e.g., up to 150° C.).

Activating soluble and insoluble ion-exchange materials with biologically active ionic agents (for illustration here, a cation (Cat)^{m+}) results in materials with two different types of solubility behavior. For example, a cation modified sulfonated poly(styrene-divinyl benzene) resin can release (Cat)^{m+}(X⁺) m in the presence of salt solutions such as NaCl (Na⁺X⁻) such as saline or physiologic fluids.

Activating an initial ion-exchange polymer salt by salt exchange to substitute biologically active ionic agents (including metallic and organic ionic or ionized compounds) may be aided by addition of heat or pressure, by the use of columnar flow reactors, and use of various solvents, as elsewhere described.

As shown in illustrative Reaction Scheme 1, the number of positive charges in the depicted ion-exchange polymer material is equivalent to the number of sulfonate groups present in the exemplary polymer. Accordingly, if the cation is a di-cation for example, it can be associated with more than a single sulfonate, carboxylate or phosphorylate group. In one exemplary embodiment, sodium polystyrene sulfonate is associated with an oligodynamic metal as shown in reaction scheme 2.

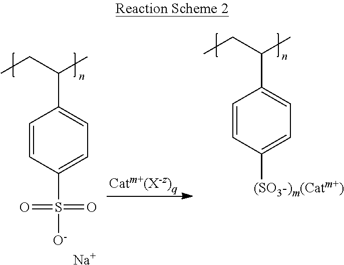

In another exemplary embodiment, polystyrene sulfonic acid-co-divinylbenzene is combined with the acetate salt form of an organic or metal cation (e.g. silver acetate, lithium acetate, zinc acetate or copper (I) or copper (II) acetate) in deionized water. The byproduct odor of acetic acid is evidence that the reaction has proceeded to yield the metal or organic sulfonate. In yet another embodiment, the acid form of polymethacrylic acid-co-divinylbenzene can be reacted with the acetate salt of a metal or organic cation to yield byproduct acetic acid and the metal or organic salt of the methacrylic acid copolymer. The biologically active, exchanged counter-ions can be “variably loaded” onto the polymer backbone to finely adjust surface activity properties of the materials and products herein, for example by associating a selectable load concentration or density of from 1% to 100% of the biologically active, substituting counter-ion with functionalizing ion (e.g., sulfonate) on the polymer backbone, leaving “non-loaded” functionalizing ions remaining associated with the original counter-ion (e.g., Na+). Furthermore, a polymer backbone may be modified to include more than one active ion, for example cetylpyridinium(+) and silver(+), zinc(++) and Ag(+), or any other combination of multiple active ions identified herein.

At least a portion of the biologically active ion-exchange polymer salt particles produced here will typically retain at least some “non-loaded” functionalizing ions. In other words, the biologically active exchange counter-ion will not be associated with the polymer at all available ion-exchangeable sites. Even while the loading of these sites with biologically active counter-ion is “variable” or “selectable” using ion-exchange chemistry methods described here, the maximum loading of fully exchange biologically active polymer salts will typically be less than or about 90% absolute ion-exchange saturation capacity of the ion-exchange polymer (e.g., 90% replacement of total available initial counter-ion, such as Na+, substituted by the biologically active counter-ion, e.g., Ag(+)) whereas protonation driven exchanges tend to yield product with closer to, or equivalent to 100% of exchange capacity loading unless molecular or atomic spacing prevents completion of the reactions. Expressed alternatively, the maximal loading of biologically active counter-ion onto selected ion-exchange materials can oftentimes range to 90-100% of a theoretical maximum ion-“exchange capacity” of the subject ion-exchange material. Within this range the materials and products of the invention can be finely tuned for selected levels of biological activity (depending mostly on the agent and specific biological activity being employed) by metered loading of the ionic active agent onto the polymer backbone. For different activities where a lower “dosage” or loading is desired (e.g., when the biologically active ionic agent is particularly potent, or perhaps toxic, or when materials may be used in contact with sensitive tissues), the loading onto the resin may be reduced to a minimum level (1-5% or 1-10% of the projected ion-exchange potential of a polymer is occupied, or intermediate ion-exchange polymer loading levels may be selected of between 10-25%, 25-45%, 45-65% or at exchange capacity (EC) that is higher and approaching maximum EC. For low potency biologically active ionic agents, or more resistant targets, higher titer loading is employed, for example in ranges of 50-70% (e.g., % of maximum ion-exchange potential, or % of initial counter-ions actually exchanged by biologically active substitute counter-ion), 70-85%, 85-90% or even higher (up to practical saturation). One alternative method can involve adding reduced amounts of fully exchanged resin. Employing the variable loading methods and materials described here, the invention provides for variable loading of ion-exchange polymer salts with different oligodynamic metals to yield variably loaded ion-exchange polymer salts comprising 1-10%, 10-20%, 20-30%, 40-50%, or greater, up to practical saturation, of the oligodynamic metal ion by weight.

In some embodiments, the functionalizing ion in the ion-exchange material may be carboxylate. As shown in Reaction Scheme 3

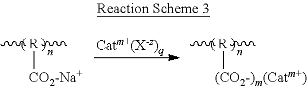

In some embodiments, the polycarboxylated salt may possess an exchange capacity of between 1.0 and 15.0 mEq/gram. The polycarboxylated salt may comprise one or more monomers of acrylic acid, methacrylic acid, vinylbenzoic acid, arylenevinyl carboxylate, or divinyl benzene. In exemplary embodiments, the polycarboxylated ion-exchange resin may be a commercially available product such as Amberlite™ IRP64 (Polacrilix resin), Dow Chemical's MAC-3 resin systems or Purolite's C106 & C107 line of weak acid cation exchange resins (polyacrylics). Amberlite IRP64 has a reported exchange capacity of 10.0 mEq/g. Biologically active counter-ions can be associated with one or more of the carboxylate groups in the ion-exchange polymer. In some embodiments, the composition may include a blend of at least two different salts of a polycarboxylated compound, whereby the cation occupies from 1% to 100% of available carboxylates. In more detailed embodiments, the biologically active cation or anion occupies from 1-10%, 10-20%, 20-40%, 40-60%, 60-80% or 80-95%, up to between 90% and complete practical saturation, of available functional groups/exchange sites as active counter-ion within the activated ion-exchange polymer salt. In some embodiments, the functionalizing ion in the ion-exchange material may be phosphate. An exemplary cation-exchange polymer of this type is cellulose phosphate. This material can be activated by antimicrobial cations such as copper (II), for example. Cellulose phosphate is a strong cation-exchange material of variable ion-exchange capacity (generally around 7 mEq/gram).

Biologically active counter-ions for activating ion-exchange polymer materials can include any number of inorganic or organic cations or anions. Counter-ions that can be readily associated (without chemical conversion to an ionic or salt form) with useful ion-exchange polymers can include one or more metal cations, organic cations, quaternary ammonium compounds, protonated amines, carboxylates, phosphates, amine containing therapeutic agents, ammonium containing antibiotics and antimicrobial agents, nitrogen containing antibiotics and/or biguanides.

In some embodiments, the cationic biologically active agent is a mono, di, or trivalent metal including, but not limited to, an oligodynamic metal cation such as silver(I)/Ag(II), copper(II)/Cu(II), zinc(II)/Zn(II), iron(II)/Fe(II), gallium/Ga or bismuth(II)/Bi(II), amenable to ionic association with a sulfonate, carboxylate, or phosphate anion, for example. In other examples, the metal cation may be one or more of a monocation species Na⁺, Ag⁺, K⁺, Li⁺, Au⁺, a dicationic species Ba⁺⁺, Ca⁺⁺, Cu⁺⁺, Zn⁺⁺, Mn⁺⁺, Mg⁺⁺, Fe⁺⁺, or a trication species such as Bi⁺⁺⁺, Ga⁺⁺⁺, and/or Ce⁺⁺⁺ or combinations thereof. In illustrative embodiments, useful materials for association by counter-ion exchange with an ion-exchange polymer salt (e.g., a polysulfonated resin salt), include, for example, a silver salt, copper (II) salt, cerium (III) salt, Gallium (III) salt, cetylpyridinium salt, benzalkonium salt, chlorhexidine salt, centrimonium (centrimide) salt, octenidine salt, zinc (II) salt, iron (II) salt, or minocycline salt, or combination thereof, as shown in the exemplary structural diagrams below.

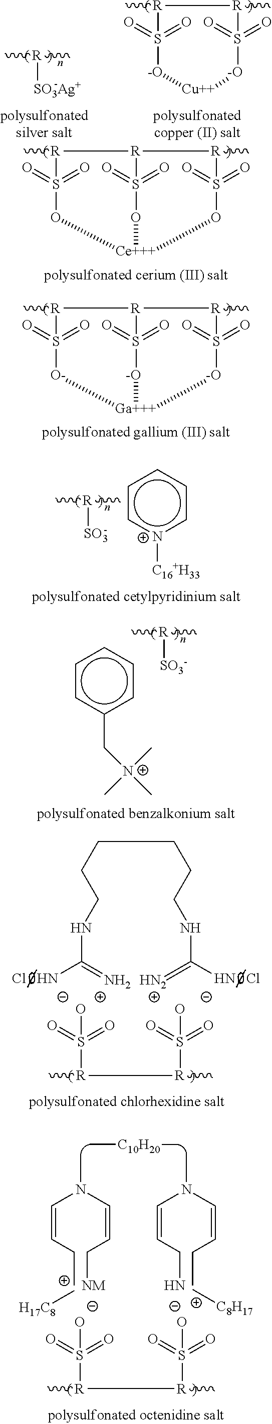

Sulfonate may also be associated with NH₄⁺, RNH₃⁺, R R″NH₂⁺, RR R NH⁺, or⁺, RRRR N⁺, for example where R represents an aryl, alkyl or mixed aryl alkyl groups or the sulfonate can be associated with a pyridinium cation. According to another example, the sulfonate group can be associated with one or more of organic species including nitrogen containing organic species such as an amino acid, a polyamine, a polyammonium ion, a tetracycline, doxycycline, arginine, gentamycin, ammonium chloride, cetyltrimethylammonium bromide, lysine, glutathione, lidocaine, albuterol, and/or alkyl/benzylammonium, pyridinium such as cetyl pyridinium, a guanidinium ion such as with chlorhexidine or polyhexanide, amino or oxazole, triazole, or thiazole containing compounds such as antifungal agents to include ketoconazole, or clotrimazole, and (dihydropyridinyl) species such as octenidine for example. In some embodiments, the copolymeric ion-exchange material may be ionically bound to a plurality of therapeutically useful counter-ions. For example, an oligodynamic metal ion and a quaternary ammonium ion may both be bound to the same copolymeric ion-exchange material. In other embodiments, more than one therapeutically useful counter-ion from the same class may be bound to the same copolymeric ion-exchange material, for example a plurality of two oligodynamic metal ions may be bound to the same copolymeric ion-exchange materials, for example to include copper (II) and zinc (II).

Useful antimicrobial counter-ion materials in the self-disinfecting compositions described herein include, but are not limited to, antibacterial drugs, antiviral agents, antifungal agents, antiparasitic drugs, as well as oligodynamic metals.

Antibiotics include, but are not limited to, natively cationic antibiotic and antibiotics that are readily protonated to cationic form (each of which can be readily associated with either polycarboxylated, polyphosphorylated, and/or polysulfonated ion-exchange polymers). Exemplary antibiotics include semisynthetic penicillin such as ampicillin and amoxicillin; monobactams such as aztreonam; carboxypenems such as imipenem; aminoglycosides including streptomycin; gentamicin; glycopeptides such as vancomycin; lincomycins including clindamycin; macrolides such as erythromycin; polypeptides such as polymyxin; bacitracin; polyenes such as amphotericin; nystatin; rifamycins such as rifampicin; tetracyclines; and doxycycline, among others.

Exemplary antiviral agents include, but are not limited to, acyclovir, idoxuridine, etravirine, and tromantadine.

Exemplary antifungal agents include, but are not limited to, miconazole, ketoconazole, fluconazole, itaconazole, econazole, terconazole, oxyconazole, grisefulvin, clotrimezole, naftifine, and polyenes such as amphotericin B or nystatin/mycostatin.

Exemplary anti-amoebics include, but are not limited to, metronidazole and tinidazole.

Exemplary antihistamines include, but are not limited to, diphenyl-hydramine, chlorpromazine, pyrilamine and phenyltoloxamine.

Useful antioxidant counter-ion materials include, but are not limited to, glutathione and carnosine.

Other therapeutically useful counter-ions may comprise ionic or ionizable chemotherapeutic and anticancer agents, such anthracycline antibiotics (e.g., doxorubicin) which can be readily associated with ion-exchange resins of the invention and incorporated in useful composites effective to treat cancer (e.g., by implantation or other delivery of the composite to a site of a tumor). Other useful biologically active agents for use as active ionic (or ionized) agents within the polymer salts and polymer composites of the invention chemotherapeutics include, for example, alkaloids such as morphine, antihypertensive agents such as verapamil sedatives and hypnotics such as benzodiazepines, anti-migraine agents such as sumatriptan; anti-motion sickness agents (such as cinnarizine); anti-emetics (such as ondansetron, adrenergics such as amphetamine; antispasmodics such as papaverine, ataractics such as benactyzine; antihypertensives such as hexamethonium analgesics such as 2,6-diamino-3-phenyl-azopyridine; antitussives such as dihydrocodeine; antipsychotics such as imipramine; coronary dilators hypotensives such as hydralazine and clonidine; and peripheral vasoconstrictors such as tolazoline, among others.

There are many examples of amine-containing drug compounds useful within the activated polymer salts and polymer composites of the invention that may include ergotamine, verapamil and nifedipine.

Other exemplary compounds with ionizable nitrogen atoms within the structure include phenylalanine mustard), procarbazine and pyridoxine; nucleosides (purines), and chitosan or polylysine for example.

Exemplary contraceptives include spermicidal agents, anti-motility agents effective to disable spermatozoa flagellar function, anti-ovulation agents, and anti-conception agents, among others. An exemplary spermicidal agent that can be associated with biologically active polymer salts and thereby integrated in biologically active polymer composites of the invention include Cholalic acid, (common name=cholate) as associated with an anion exchange resin and anti-conceptive materials and formulations (optionally in conjunction with benzalkonium chloride) as associated with a cation exchange resin.

In certain embodiments, spermicides and/or other contraceptive agents are incorporated in the biologically active polymer composites of the invention, which are fabricated to provide functionally and/or anatomically formed contraceptive devices. In one exemplary embodiment, intrauterine contraceptive devices (IUDs) are provided combining an activated polymer composite incorporating a copper derivative associated with an activate polymer salt particulate embedded in the composite (e.g., in a form SO3⁻, CO2⁺, OPO3⁻). Comparable active agents and polymer salts can be readily incorporated in vaginal sponge and cervical diaphragm devices, for example, formed partly or entirely from activated polymer composites of the invention. These novel devices are constructed to deliver spermicidal and/or anti-conceptive ionic copper at a surface of the IUD device, sponge, condom or diaphragm, or in other embodiments to deliver a metered or titered dose (i.e., a spermicidal and/or anti-conceptive effective amount) of solubilized ionic copper to a target site, such as a vaginal, cervical or uterine compartment, to mediate effective contraception (often by activatable dissociation/solubilization of the active ionic agent triggered by contact of the activated composite surface with an aqueous ionic fluid, e.g., physiological, fluid).

These and other aspects of the invention described herein may be clarified in reference to prior, related disclosures in U.S. patent application Ser. No. 16/568,071, filed 11 Sep. 2019; U.S. patent application Ser. No. 16/269,426, filed 6 Feb. 2019; U.S. patent application Ser. No. 16/159,593, filed 12 Oct. 2018; U.S. patent application Ser. No. 15/896,016, filed 13 Feb. 2018; U.S. patent application Ser. No. 15/509,834, filed 8 Mar. 2017; PCT International Patent Application No. PCT/US15/49255, filed 9 Sep. 2015, and U.S. Provisional Patent Application No. 62/047,655, filed 9 Sep. 2014, each incorporated herein by references for all purposes. In other novel embodiments of the invention, catheter hubs (FIG. 12), the connector portion of the catheter that allows for the connection to a drug reservoir such as for chemotherapeutic delivery or a dialysis device for purifying blood can be rendered antimicrobial to aid in alleviating central line-associated blood stream infections. The connector can be injection molded from biologically active polymer composites of the invention, for example using a matrix material selected from polycarbonate or acrylic. The composite may comprise an active silver ion or a copper (II) ion exchange resin for example. By using copper (II), in addition to the known antimicrobial effect of Cu(II) alone, the surfaces can be cleansed by the addition of dilute sodium chloride in water (saline or >0.9% NaCl, 154 mEq/L) and H₂O₂. In this example, at neutral pH NaCl_{aq} facilitates Cu(II) release and H₂O₂ acts on the copper ion as described in equations (1-3) to liberate hydroxyl radical (HO·): 1. Cu(II)+H₂O₂ ┘Cu(III) HO·+OH⁻, 2. Cu(III)+H₂O₂┘Cu(II)+HOO·+H⁺3. 2H₂O₂+HO·+HOO·+H₂O. HO· has been shown to be effective at disrupting certain biofilms. To deliver the aqueous saline H₂O₂ combination to a luer-type active composite catheter hub, a sponge-fitted, mating cap incorporating saline with H₂O₂ for example may be connected to the hub to amplify disinfection. These embodiments achieve qualitative production of hydroxyl radical on the surfaces of epoxies, PVC and other materials, e.g., using a methylene blue bleaching test (Satoh, A. et. al., Environ. Sci. Technol. 2007, 41, 2881-2887). 12 This approach to generating hydroxyl radical decontaminant may be used in other embodiments, for example to produce mouth pieces, dental materials and implants resistant to harmful biofilms, and for producing coatings that inhibit biofilm, fabrics and the like for use in vehicles, plumbing (p-traps) and air circulation ductwork and filters, clinical surfaces, tools and equipment, and to protect or decontaminate personnel exposed to biological contaminants, chemical warfare agents and other contaminants. Environments where this may be important include water storage and water-recycling/reuse systems found within the international space station/vehicles for space travel, submarines, ships and the like, for example. In certain embodiments, the use of a Cu(II)-SCER modified coating, such as an epoxy coating, will be sufficient to prevent contamination and pathogen transfer safely and effectively. Notably, it is more efficient if Cu(II) is freed from the composition for the Fenton reaction to be optimally effective. To accomplish this, some cations (e.g. Na⁺) are necessary. Drinking water is generally much lower in Na⁺ than saline, as such the Fenton reaction will be much slower in drinking water thus preserving longevity. In these water processing and storage systems, coatings that incorporate the fine particulate biologically active ion-exchange polymer salt material can be applied to stainless steel holding tanks as powder coatings for example. Powder coatings can include but are not limited to polyurethanes, fusion bonded epoxies, and polyesters.

In yet another embodiment, PVC plumbing piping (e.g. P-trap) when injection molded from PVC formulated to include a copper (II) ion exchange resin can be used to provide protection against microorganism growth, particularly antimicrobial-resistant microorganisms when building clinical institutions. For disinfection, the drains can be treated with a mixture of saline and 3% (or stronger) H₂O₂ to initiate a Fenton reaction.

In a simple embodiment of a water purification device, water removed from a source (river, lake, spring, etc.) can be filtered through a high surface area (reticulated) filter fabricated from an active polymer salt particulate embedded within a polymer matrix (to provide an active polymer composite) into a container containing a high surface area sponge-like substrate fabricated from or coated with, an active polymer salt particulate embedded within a polymer matrix. Bacteria that adhere to the surface will be rapidly killed and the remaining organisms will be killed with surface exposure. Other novel embodiments include dental fillings comprising a light activated or chemically vulcanizing formulation of an acceptable filling polymer such as an acrylic compounded to include a biologically active antimicrobial ion exchange resin derivative such as, but not limited to, silver ion-modified strong cation (Ag-SCER) or weak cation Ag-WCER), or a zinc-based ion exchange material such as Zn-SCER or Zn-WCER exchange resin. The resulting filling material can inhibit the action of dental bacteria known to be causative of dental caries.

Other novel embodiments include catheters, and catheter-like devices that used in medical and other fields, which are known to present high infection risks. These include, urinary catheters, retention balloons and ureteral stents for example. In the urinary tract, the composites are intermittently exposed to urine. Urine has a sodium concentration generally ranging from 20-220 mEq/L. Silicones, PVC, and polyurethane, and TPE (styrene-ethylene-butylene-styrene) Normal saline (0.9% NaCl) is closer to the midpoint of this range at 154 mEq/L. This environment is conducive to the exchange of Na⁺ for Ag⁺ and the formation of a fine crystalline layer of AgCl on the surfaces, as evident by scanning electron microscopy, of the catheter. These protective layers preclude the formation of biofilm. This may explain the (only) slight differences observed in efficacy between artificial urine and pooled human urine (HPU, FIG. 10). In HPU, a 5-log reduction (24-hrs.) was observed vs. control silicone.

Within other novel embodiments of the invention, biologically active polymer composites will incorporate ionic or ionizable forms of anticoagulants and other hematologically active compounds useful to prevent or facilitate blood clotting, inflammation, atherosclerosis, restenosis, stroke and other adverse sequelae associated with vascular and coronary pathologies, and/or with conventional use of vascular stents, shunts, grafts (artificial and autologous), prostheses or implants (e.g., coronary valves, pacemakers and electrodes). In exemplary embodiments, a low molecular weight heparin such as Dalteparin can be effectively bound to weak and strong anion exchangers and employed within composites of the invention incorporated within these biomaterials and devices as described, to prevent clotting and/or restenosis after vascular or coronary surgery. Additional embodiments will employ Cloricromen, a platelet aggregation inhibitor. Other embodiments will employ Benzamidine-based thrombin inhibitors such as α-NAPAP (N-alpha-(2-naphthylsulfonylglycyl)-4-amidinophenylalanine piperidide). Direct Thrombin Inhibitors such as Dabigatran (Ethyl-3-{[(2-{[(4-{N-hexyloxycarbonylcarbam-imidoyl}phenyl)amino]methyl}-1-methyl-1H-benzimidazol-5-yl) carbonyl](pyridine-2-yl-amino) propanoate) are similarly useful within anti-clotting, anti-sclerotic, anti-thrombotic, anti-restenotic, anti-stroke, anti-arrhythmic, and anti-coronary arrest biomaterials, devices and methods, among other related compositions, implants, apparatus and methods. Other embodiments include loading of the sodium form of the strong cation exchange resin into a medically relevant polymer to be used within the arteriovenous system. Polysulfonic acids are known to possess anticoagulant properties and as such composites incorporating these particles at the surface will display a resistance to clotting. Additional embodiments of the invention employ peptide-based therapies, with biologically active composites incorporating ionic or ionizable peptides, peptide fragments, peptide conjugates and other useful peptide drugs and compositions. Peptide drugs can be challenging to deliver given their susceptibility to the gut and to proteases that can degrade activity. Small peptides can be associated with biologically active ion-exchange polymer salts according to the teachings herein, and these can be formulated within polymer composites in a wide array of useful biomaterials and devices. In one exemplary embodiment, a peptide active agent is incorporated in an activated polymer composite of the invention as a vaginal, colonic or oral sponge, capsule, implant or particulate suspension for delivery of the active peptide to a highly vascularized mucosal tissue of the vagina, uterus, lower colon or rectum, or oral mucosa. Other mucosal peptide delivery forms include nasal delivery composites that employ non-crosslinked ion exchange materials.

In certain embodiments, the fine particulate polymer salts can be synthesized as biodegradable resins to permit release into the environment, or to permit safe intranasal or intrapulmonary delivery of active particulate aerosols to carrying therapeutic ionic active agents to target tissues of mammalian subjects, where the ionic agents may be released (dissociated and solubilized from the polymer salt carrier following contact with a physiological ionic fluid) or mediate surface active drug, antimicrobial or therapeutic activity.

In additional aspects of the invention, a wide range of orthopedic biomaterials and devices will beneficially incorporate activated polymer salts and polymer composites of the invention.

Among many orthopedic uses contemplated, posts of implants are known to be high-risk conduits for entry of microbial infectious agents into hip implant patients. The invention provides a variety of useful composites to prevent this contamination/infection risk, including epoxy, silicone, and acrylic plugs compounded with sulfonated polystyrene-divinylbenzene-tobramycin or gentamicin salt (or polymethacrylic acid-divinylbenzene-tobramycin or gentamicin salt) for placement at a site of a hip implantation post. These composites and devices provide effective slow release of ionically associated drug over time. In more detailed embodiments, these composites (generally useful for adjunctive application with a diverse array of prosthetic implants, including dental and surgical posts, pins, anchors, sutures, stents, etc.) are often formed as a porous solid composite (e.g., spongiform, lattice form, open cellular, blown or extruded composite), which can be facilitated by addition of any of a variety of known useful polymer foaming agents—to increase surface area for enhanced drug delivery (i.e., with faster kinetics or higher doses of drug delivered, and more effective sustained delivery—e.g., with effective delivery amounts maintained for 1-3 days or weeks, 1-3 months or longer). Polyurethane-based polymer composites described herein are particularly amenable to fabrication of foams. The pores are the result of the liberation of CO₂ resulting from the addition of water during the cure process.

Other novel embodiments of the invention include thin adhesive films of biologically active polymer composites that can be applied to touch surfaces in high traffic community areas for example. Such locations can include airports, train stations, grocery stores and the like. Application specific locations may include cart handles, door handles, bathroom locations, x-ray equipment touch points, buttons, seating area components such as armrests, and other surfaces that may act as fomites for pathogen transmission. The thin adhesive films of biologically active polymer composite, once applied to a substrate, can be activated to aid disinfection by the administration of an aqueous ionic fluid such as tap water or saline. When the thin adhesive films of biologically active polymer composite comprise Cu(II)-SCER, the addition of hydrogen peroxide to an aqueous ionic fluid such as tap water, saline, or quaternary ammonium disinfectant can greatly enhance disinfection.

In additional aspects of the invention, biologically active polymer composites can be used in the fabrication of ventilation system components such as filters that can reduce or eliminate viral pathogens from circulation. Ventilation system that can reduce or eliminate threats such as SARS-COV-2 is an important element of readiness particularly as it pertains to military vehicles to include ships, submarines, and aircraft. Navy ventilation systems utilize steel, aluminum, copper, fiberboard, and other polymeric components. Ductwork and metal prefilters for ship ventilation systems are largely comprised of aluminum and/or galvanized steel. Several filter types are found on Navy ships and most common is the Navy Standard metal mesh filter, manufactured of aluminum or steel. These filters are typically cleaned onboard. In some instances, they may be coated with a tacky polymer. Disposable filters, including chemical, biological, and radiological (CBR) filters, and HEPA filters are also used. There is a dearth of literature to describe submarine ventilation systems. It is understood that the ductwork is comprised of metal and it is likely there are similarities to ships. Generally, metallic (mesh) prefilters are used in combination with synthetic and HEPA filters.

Aircraft ventilation systems are different because the mass of the ventilation system is important. The only portions that are comprised of metal are the high-pressure inlet components.¹⁵ The remainder of the system uses a variety of composites of epoxy, silicone, phenolics, polyimide and at some locations of flexible tubing materials in order to address turns in the system.

The U.S. Navy maintains approximately 299 active deployable battle force ships and the U.S. Airforce maintains approximately 5,047 manned aircraft. These vehicles require the support of significant “human” resources. For example, an aircraft carrier can house 3,000-6,000 personnel. The presence of this many individuals aboard a single vehicle, that has a footprint in many ports around the globe, can pose a threat to military readiness in the event of an outbreak of virulent disease.

In March of 2020, an outbreak of Covid-19 caused by the SARS-COV-2 virus was detected on the USS Theodore Roosevelt Aircraft Carrier and it was determined that the virus was transmitted through the ventilation system. On Apr. 1, 2020, the Navy ordered the carrier to be largely evacuated leaving a skeleton crew of 400 to maintain the ship while it was decontaminated. Hundreds of shipmen had become infected and 1-fatality resulted.

In certain embodiments, biologically active polymer composites or polyethylene incorporating Cu(II)-SCER are meltblown and spunbond into nonwoven fabrics for use in ventilation system filters or personal protective equipment such as protective surgical masks, N95 masks, surgical gowns, surgical clothing and curtains. The benefits of organic “strong” and “weak” cation exchange Cu(II)-based ion exchange resins includes improved mixing compatibility with organic polymers, their generally higher exchange capacity than mineral exchangers and the ability to bind larger cations given the restrictions of mineral cavities. The Cu(II) additives have been shown to provide safe and controlled Cu(II) release from surfaces at concentrations below EPA levels established for safe drinking water (1.3 ppm). These materials can be deployed as components for use within ventilation systems to reduce microbial and viral pathogen transmission. Incorporation of the additives into melt blown fabrics (e.g. polypropylene) and spunbond fabrics (e.g. polypropylene) allow for high efficiency and lower efficiency (rough) filtration. Melt blown fabrics consist of smaller diameter filaments, including submicron filaments, and have superior filtration properties to other nonwovens. The meltblown process includes hot air being blown onto molten thermoplastic resin that is extruded through a linear die containing hundreds of small holes, to form a fine fibered self-bonded nonwoven web. Its key feature is that it is an extremely thin fiber. As a result this material is often used for filters for air, liquids and particles, or as absorbents in products such as wipes, oil absorbents, incontinence products, and female hygiene, but can also be used in the production of certain electronics, adhesives, and other apparel. Conventional spunbond fabrics contain coarser fibers and have much greater tensile strength and smaller pressure drop. More specifically, the incorporation of these novel resins into meltblown polypropylene (PP) nonwovens is anticipated to allow us to fabricate high efficiency filters to remove heterogeneous pollutants, microorganisms, and viruses with the intent of preventing transmission. The effort proposed here targets the development of antiviral and antimicrobial meltblown nonwovens with fine fiber diameters of 2-4 μm and a Minimum Efficiency Reporting Value (MERV) of 13; the low end of high efficiency. Among the major markets for spunbonded materials are hygiene cover stock, medical fabrics, geotextiles, construction and carpet underlays. Melt blown materials primarily target the hygiene, filtration and industrial segments. Both are made from an integrated process of spinning, attenuation, deposition, bonding and winding. Melt blown fabrics tend to have a lower fiber denier than their spunbond relatives. Furthermore, melt blown fabrics are composed of discontinuous filaments unlike spunbond materials. Spunlace nonwoven materials are an environmentally friendly fabric processed using high pressure water entanglement to form the web.

When melt blown and spunbond materials are layered to form a spunbond-meltblown-spunbond (SMS) material the features can be combined together, and the range of applications increases.

Additionally, the combination of spunbond and meltblown materials means that the features of each can make up for the weaknesses of the other. For example, meltblowns have limited strength so can be combined with a spunbond to become a strong material, and likewise spunbonds can be elevated with the addition of meltblowns. These can even be combined to make a material which has a textile feeling to it. SMS has excellent physical properties as well as barrier qualities. Features include high tensile strength, softness, comfort, breathability, wearability, and is also lightweight. It also acts as a water-repellent, and a barrier against bacteria, blood and other liquids as well as gas/steam perspiration. Finally, it is also fine enough to serve as a disposable fabric.

Medical SMS Fabric is suitable for medical and hygiene products such as diapers, protective wear including surgical and N95 masks (face masks), hospital gowns, wound care, caps, filtration fabrics, surgical drapes and more.

Although SMS is more expensive than other materials such as non-layered materials, its unique features and specific design make it widely applicable for use. It can not only mimic the appearance, texture and strength of a woven fabric and can be as bulky as the thickest padding, but in combination with other materials also provides a spectrum of products with diverse properties. With the development of SMS it is thought that it will grow rapidly in the market of nonwovens and become more and more popular for various segments including apparel, geotextiles, furnishings, roofing as well as many others.

With the inclusion of Cu-SCER, Ag-SCER, Zn-SCER, quaternary ammonium-SCER or other fine particulate biologically active ion exchange salts, antibacterial, antifungal, and antiviral fabrics result. In some instances, a non-toxic/benign carboxylic acid (sacrificial) salt can be added along with the biologically active ion exchange salt. The addition of this sacrificial salt during the processing of the material into fabric facilitates ion exchange in a finished (fiber)/fabric with the addition/presence of nonionic humidity and/or water such as from breathing.

In one embodiment, an N95 mask can be manufactured. Because breathing within the mask renders the atmosphere humid and because this humid environment is devoid of ion content that enhances exchange with the embedded biologically active ion exchange salt, the addition of a sacrificial salt such as, but not limited to those of a phosphate, a carbonate, a formate or an acetate, i.e. salt of formic acid or acetic acid) can facilitate exchange with the biologically active ion exchange salt. As humidity can slightly solubilize the sacrificial salt, exchange with the biologically active ion exchange salt occurs. In the case of formate, for example, very small quantities of Cu(II) formate are liberated. Cu(II) formate is antibacterial, antifungal and antiviral. The sacrificial salt must possess a melting point above the processing temperature to generate the fabric. Examples of sacrificial salts include but are not limited to sodium formate (melting point >485° F.) and sodium acetate (melting point ~615° F.), allowing processing of the fabric at temperatures easily between 450° F. and 550° F. In the presence of moisture, as from breathing, Cu(II)-SCER for example can exchange with sodium formate (HCO₂Na) according to the equation: 2 HCO₂Na⁺Cu(II)-SCER+H₂O→Cu(HCO₂)₂+Na₂-SCER. This phenomenon has been demonstrated using an epoxy incorporating Cu(II)-SCER and sodium acetate. With exposure to deionized water (which is incapable (alone) of releasing Cu(II) from Cu(II)-SCER, Cu(II) was measured within seconds of exposure to deionized water. This indicates the presence of Cu(II) acetate. The use of this, and other compositions, for spunbond and melt blown fabrics readily allows for the fabrication of specialty medical goods that possess antibacterial, antifungal, and antiviral characteristics.

In yet another embodiment, nonwoven ventilation system filtration components can be fabricated using such a method and prefiltration aerosolization of small quantities of sterile deionized water or hydrogen peroxide can enhance the ability of such filters to capture and inactivate viruses particularly with the liberation of hydroxyl radical at the filter level. In yet another embodiment, the nonwoven ventilation system filtration components of the current invention can be paired with an electrostatic precipitation component to aid in virus particle collection. To further improve the ability of this system to inactivate virus from an airstream, a copper-based metal prefilter can act as one of the electrodes of the ESP system. The copper-based electrode may be comprised of a copper alloy or cold spray copper surface for example. The success of a Cu(II)-SCER composition against Coronavirus 229E, an enveloped virus surrogate for SARS-COV-2 enables inactivation of enveloped viruses in ventilation systems using filters incorporating biologically active ion exchange salts.

Another embodiment of the present invention utilizes Cu(II) modified clay particles, Cu(II) modified zeolites, and other Cu(II) modified naturally occurring and synthetic minerals that can be incorporated into nonwoven materials. This inventive concept can be applied beyond nonwoven materials to include paints and coatings compositions as described in this present invention.

Many useful drugs and other therapeutic agents that do not natively exist in an ionic form, or which are not available in a useful salt form to provide for preparation of ion-exchange polymer salts, can be rendered into such useful forms by a variety of chemical processing and/or chemical modification methods. Methods for generating drug forms amenable to binding to anion exchange polymer materials, for example, include formation of carboxylate (CO₂—) by hydrolysis of esters (CO₂R), where R is generally an alkyl group. In comparably useful processing methods, sulfonates can be generated in the same fashion (although sulfonic acid esters can be alkylating agents and therefore mutagens or carcinogens when encapsulated into a polymer matrix, particularly hydrophilic matrices wherein the sulfonic acid esters can hydrolyze to release a hydroxyl-terminal component of the ester). One method for the delivery of hydroxyl-terminated drugs therapeutic agents) involves the formation of the sulfonic acid ester of a strong cation exchange resin such as IRP69 (IRP69-SO₂—OR) where OR represents the hydroxyl-terminated active agent. In the presence of water, within a polymer matrix, such as a matrix designed to absorb water (a hydrogel for example), the sulfonic acid ester is hydrolyzed to yield the hydroxyl-terminated active agent (HOR) plus the sulfonic acid of the organic ion exchange resin (IRP69-SO₃H). One such active agent for functionalizing the resin is dexamethasone by reaction with the sulfonic acid chloride. Similar chemistry can be applied to phosphate esters as well, as these compounds can be hydrolyzed in similar fashion. In a similar embodiment, the sulfonated resin is converted to the sulfonic acid chloride and the modified resin reacted with a fatty amine such as octadecylamine. After milling, the resin can be incorporated into materials to alter the surface hydrophobicity.

Additional embodiments of the invention provide effective hemostat compositions that prevent bleeding. Exemplary hemostat compositions employ epinephrine, a hormone and neurotransmitter produced by the adrenal glands, exchanged onto the backbone of a strong or weak cation exchange resin rendered as a fine particulate as described and loaded into a textile or foam, coated onto a high surface area fabric with incorporation into a lacquer, incorporated directly into a nonwoven substrate (fabric) formed by a spunbond process, or as a compression dressing for application to a bleeding wound, or a strong or weak cation exchange resin modified to include bound chitosan and after milling is subsequently incorporated into configurations as described above to include polyurethane foams, nonwovens, or compression bandages/dressings. Many configurations of high surface area constructs can be envisioned that can are formed by coating a variety of fabrics and spunbond composite materials of various polymer compositions and fiber diameters. In related embodiments, anesthetics such lidocaine, novocaine, mepivacaine, tetracaine, benzocaine, bupivacaine, ropivacaine, or articaine are also loaded onto the resin backbone and incorporated into the polymer matrix, textile, foam or other material or device for therapeutic application onto or within wounds (e.g., traumatic or surgical wounds, burn sites, etc.). The anesthetic will improve efficacy and tolerability of hemostatic and antimicrobial dressings and enhance local pain management.

In additional hemostatic embodiments, calcium salts of a strong or weak cation exchange resin (in powder form) can be combined into a hydrophilic large pore, high surface area open cell foam and resulting dressings deployed as hemostatic devices. Blood coagulates in pores of the dressing material or device, resulting in clotting and stoppage of blood flow.

In an alternative embodiment to a hemostat, a strong anion exchange resin such as Purolite A502PS is milled to small particle size and added to a polyurethane foam matrix to allow for the preparation of a thin flexible sheet that can be pressed into a wound to prevent blood loss. The quaternary ammonium groups interact with negative charged sites on membranes of erythrocytes, mediating bioadhesion that facilitates coagulation and achieves hemostasis. In each of the foregoing hemostatic embodiments, a range of forms can be employed as described for the production of wound dressings, coatings, compresses or other hemostatic devices, for example including woven and nonwoven materials (e.g. spunbonded and melt-blown nonwovens) made using a spunlaid method employing polypropylenes and other materials in the form of solids. In exemplary embodiments, modified nonwoven fabrics can be used for filtration and polyurethane foams incorporating antimicrobial and/or hemostatic ion exchange resins can also be utilized for beds and pillows in hospitals, nursing homes, corrections institutions, and other large scale housing and care facilities.

Add Information about Ventilation Systems

In certain embodiments of the invention, active ion-exchange polymer salt particles are further processed to achieve size reduction from an original ion-exchange particulate size. Typically, this size reduction processing involves fracturing of the original ion-exchange particle, but this can be achieved also by mechanical cutting, shearing, grinding or erosive techniques. Particle fracturing can be achieved using a variety of particle size reduction/milling methods. Briefly, the starting ion-exchange material (before activation) can be in the form of particles ranging from about 100 μm to about 2,500 μm in average diameter, often in the range of 500 μm to 1,500 μm. In various embodiments, it is desired to achieve substantial size reduction of these particles by milling to generate a fine particulate, activated organic ion exchange polymer salt or resin material. Desired size ranges for these materials range from about 10 nm to about 100 μm in average diameter. In certain embodiments the average particle diameter of the fine particulate, activated organic ion exchange polymer salt or resin material will be from about 100 nm to about 10 μm after size reduction. In other detailed embodiments the fine particle diameter will range from about 100 to about 700 nm. Desirably, the fine particulate milled, activated ion-exchange polymer salt material will demonstrate a desired uniformity of particle size depicting a Gaussian distribution as determined by laser particle analysis). While different methods, apparatus and compositions for milling may be used for different embodiments and aspects of the invention, one exemplary mode of milling of the activated ion-exchange polymer salt particles employs high energy milling, for example using centrifugal/planetary ball milling methods, compositions and devices. Within more detailed embodiments, high-energy milling is combined with a porous construction design of the ion-exchange polymer salt particles prior to milling. In exemplary embodiments, ion-exchange polymer salt particles may be provided with a microporous construction, wherein individual particles define small channels, voids and pore spaces within the body of the resin particle (the pore spaces and channels being surrounded by walls or partitions of the polymer salt material). After the porous polymer salt particles have been biologically active by salt exchange with the biologically active ionic agent in aqueous media, the particles are dried to remove some or all of water present in advance of milling. Subsequently the biologically active porous ion-exchange polymer salt particles are milled by a high energy milling process to render the fine particulate biologically active ion-exchange polymer salt particles as described.

In an exemplary embodiment, high energy milling of active, porous organic ion exchange polymer salt particles is conducted dry using alumina media to generate particulates in the 1.0 micrometer size range. In a secondary exemplary embodiment, the milling step is conducted with the addition of a non-solvent liquid such as water or a long chain (higher) alkane that presents reduced risk with some heating. The non-solvent liquid is added to occupy channel, void and pore spaces within the polymer salt particles. It has been discovered here that using these novel high energy milling materials and methods, the non-solvent liquid mediates size reduction of the polymer salt particles in an unexpectedly efficient and uniform manner, i.e. colloidal milling. The resulting product of this and equivalent high energy milling processes and formulae provided by the invention, is a novel, fine particulate biologically active ion-exchange polymer salt material, having an average milled particle diameter between about 10 nm to 100 μm, often as small and uniform as from 100 nm to 10 μm, and in in certain embodiments ranging between about 400 nm to 600 nm (for example having an average fine particle diameter of 500 nm).

In one illustrative milling protocol provided here, larger porous activated ion-exchange polymer salt particles are placed into a stainless-steel container lined with a hard ceramic, such as zirconium oxide. A non-reactive, high boiling and preferably non-flammable organic liquid and suitable milling media (for example barrel- or ball-shaped, ceramic milling media, such as zirconium oxide bearings) are added to the stainless-steel container. The mixture is then subject to colloidal milling. In some embodiments, the resulting particles are further processed through multi-stage milling, for example using zirconium oxide milling media of decreasing size.

Once the milling is complete, a homogeneous composition of fine particulate, biologically active ion-exchange polymer salt particles is obtained (often after separation of the particles from the grinding media and the non-solvent liquid (if employed) by evaporation. The milling media is generally separated by mechanical separation, e.g., sieving). This activated, fine particulate ion-exchange polymer salt product has been shown to be cosmetically acceptable, with excellent biological activity potential (e.g., antimicrobial character) over a broad range of weight % loadings of the starting ion-exchange polymer salt with biologically active substitute counter-ions.

In more detailed examples of high energy milling, porous activated ion-exchange polymer salt particles are size-reduction milled by high energy milling with milling media and a non-solvent liquid (typically in a sealable milling container, but alternatively in high-throughput, pass-through milling apparatus). In some embodiments, the sealable milling container has a liner made of suitable material of comparable hardness as the milling media, for example a ceramic lining adapted for ceramic milling media. The milling media, for example zirconia or alumina, beads may be in any suitable size from about 0.1 mm to about 10 mm in diameter, about 0.5 to about 5 mm, about 1 to about 5 mm, about 4 to about 5 mm, about 0.5, to about 1 mm. The non-solvent liquid may be any low volatility liquid inert to the resin and the biologically active agent to include deionized water. In certain embodiments, the non-solvent liquid is an organic non-solvent such as a long chain (higher) alkane. Exemplary long-chain alkanes can include decane among other known alkanes with suitable boiling points.

The non-solvent liquid fills the voids within and between the porous ion-exchange polymer salt particles (and interstices between these particles and milling media) and functions to oppose compression of particle structures (particularly walls and partitions of voids, pores and channels) from impact, shear, friction, pressure and other mechanical forces during the milling process. To effectuate this efficient and uniform particle fracturing, the milling container may be filled to ⅓ of its volume with the porous activated ion-exchange resin particles, and to roughly a remaining ⅔ of its volume with the milling media. This leaves approximately ⅓ of the container volume available as interstitial space between milling media units, within the interstices between polymer salt particles and media, and within porous depressions, voids and channels of the activated ion-exchange polymer salt material. This approximately ⅓ remaining volume within the milling chamber of the container is filled with the non-solvent liquid to fill the interstitial and void spaces and channels as described. In certain embodiments the method does not involve a non-solvent and utilizes dry grinding media only, thus precluding a drying step to remove the non-solvent. However, milling with a non-solvent (i.e. colloidal milling) can yield particles closer to 100-200 nm hence this can be a valuable approach to producing composites with smaller particulates.

A non-solvent can render the porous ion exchange particles non-compressible to impact, shear and other forces during milling—resulting in highly efficient and uniform fracturing and rupture of the particles to a final milled average size and size variation as described. This milling process for fine particulate biologically active ion-exchange polymer salt material polymer salts is additionally aided by controlling milling temperature. Here, milling apparatus and methods are selected which provide for a controlled milling temperature in a range from about 70 to about 95° C., often between about 75 to about 90° C., and in exemplary embodiments from about 82 to about 87° C., or approximately 85° C. In certain embodiments of the invention, artificial heating of the milling chamber is not required, rather heat generated by high energy milling friction passively controls the milling temperature (adjustable by controlling milling speed, media composition and size, non-solvent liquid selection, etc.) within a selected range of from about 70 to about 90 degrees, about 75 to about 90 degrees, or other adjustable milling temperature ranges, for example at or about a target milling temperature of 85 degrees or 90° C.

Using these and other exemplary high energy milling methods, apparatus and formulae, fine particulate ion-exchange polymer salt materials for use within the invention can be routinely produced with desired particle diameters between about 10 nm to 100 μm, about 30 nm to about 50 μm, about 100 nm to about 10 μm, about 200 nm to about 1 μm, or about 400 nm to about 600 nm, for example. In some embodiments, the material is milled to a uniform particle size of about 200 nm, 400 nm, 600 nm, or 800 nm. In other exemplary embodiments, an average particle diameter of 500 nm is provided, with very low particle size variation as described. Each of the specified, distinct particle size values described here corresponds to novel biological activity potential for the fine particulate ion-exchange polymer salt materials, and for polymer composites incorporating these novel materials. This degree of particle size selectability and uniformity is not obtainable with other milling methods, such as dry milling methods—in part due to the tensile strength, elasticity and compressibility of ion-exchange polymer salts under ordinary milling conditions.

In certain embodiments, targeted milling size distributions possess larger standard deviations for a first reduction, e.g., from particles as large as 5000 microns (with ±5-10 microns as an exemplary standard deviation, in other embodiments between ±2-7 micron, or between ±1-3 microns or lower) while following a second reduction step final particle size may average 500 nm average diameter with a standard deviation of approximately ±0.75 microns (in other embodiments lesser than or equal to ±0.50 microns, or lesser than or equal to ±0.25 microns). Ideally, a uniform distribution of small particles is achieved in a single milling step of short duration.

Once the fine particulate activated ion-exchange polymer salt particles are milled to a desired size, they are isolated if required (e.g., separated from milling media and non-solvent liquid). In exemplary embodiments, ceramic or other milling media may be removed by mechanical separation, such as sieving. Non-solvent liquids may be removed by any means generally used, most often involving evaporation. In some embodiments, due to the volatile nature of some non-solvents, this liquid is removed by controlled evaporation (to prevent harmful release of evaporated solvent into the environment, and to prevent “bumping” of the fine particulate ion-exchange polymer salt material during drying. Controlled evaporation may be conducted in a static or vacuum oven depending on the volatility of the solvent. An additional milling step of the dried material may be required to loosen the resulting particles that may have adhered together during the drying step. In some embodiments high energy milling is a multi-stage process, for example where milling is repeated 2 or more times with successively smaller sized milling media to achieve a desired particle size. The same or different grinding media and the same or different non-solvent liquids may be used in successive milling stages as required to achieve appropriately sized fine particulate ion-exchange polymer salt products as described.

Functionalized anion- or cation-exchange materials are reversibly or non-reversibly associated with a selected, anionic or cationic biologically active agent by various operable methods and formulae for ion-exchange chemistry. Typically, the selected ion-exchange polymer (functionalized and associated with initial counter-ion, e.g., Na+ for cation-exchange examples, as described) is placed in an aqueous medium in a particulate form and combined with the replacement, biologically active counter-ion (typically added to the aqueous medium as a salt form of the biologically active agent (e.g., silver acetate). Combining the particles of ion-exchange polymer material with a salt comprising an antimicrobial cation, for example, in an aqueous medium will mediate salt-exchange of the antimicrobial cation for the initial counter-cation present on the ion-exchange polymer—to yield an antimicrobially activated polymer salt derivative (having the antimicrobial cation ionically associated with the polymer). Typically, these salt-exchange processes will render the newly associated, biologically active counter-ion effectively insoluble in water (i.e., the active counter-ion will not freely dissociate in distilled water).

This insolubility or non-dissociability can be controlled to allow for partial solubility or dissociability of the active counter-ion from the activated ion-exchange polymer salt, for example by using weaker ion-exchange materials, multivalent active counter-ion agents, and other methods. Thus, in certain embodiments of the invention, the biologically active counter-ion agent may be partially soluble in ionic aqueous media, or may be completely, reversibly associated with the ion-exchange polymer such that it is insoluble in distilled water and other non-ionic media, but rendered freely soluble in ionic media such as saline and physiological fluids. In this manner the biologically active polymer salts and related composites of the invention can function in multiple activity modalities. In primary activity modality, the activated polymer salts and composites exert their biological effects mostly as “surface activity”, where the biologically active ionic agent functions primarily at a surface of the polymer salt or composite, without appreciable (e.g., greater than 5%) dissociation (typically solubilization) of the active ionic agent from the surface.

In an alternative or combined modality, the activated polymer salts and composites can also exert “non-surface” biological effects as drug delivery materials or devices, wherein in addition to “surface activity” the biologically active ionic agent is also “reversibly-associated” with functional groups on the ion-exchange polymer salt materials in the composites. They are therefore ionically dissociable from the composite surface under certain conditions and can be released in a soluble form following exposure to, e.g., ionic aqueous media including physiological fluids. In these aspects of the invention, polymer composites incorporating activated ion-exchange polymer salts function as drug and active agent delivery materials and devices—i.e., to deliver dissociated, biologically active ionic agents to tissue and compartments adjacent to or distant from the polymer salt/polymer composite surface. For example, an Ag-SCER silicone composite, the release of Ag(+) in its salt form (e.g. AgCl) can be controlled by the solubility of the resulting species. AgCl possesses a Ksp of 1.8×10⁻¹⁰ and with its release precipitates onto the surface of the composite in microcrystalline form FIG. 3). The presence of AgCl on the surface is protective against microorganism proliferation and further ensures that AgCl has achieved its solubility limit in that environment thus precluding the release of additional Ag(+) to form AgCl as a result of the equilibration of Ag(+) associated with the resin and precipitated AgCl. This equilibrium provides high local surface concentrations of dissolved Ag(+) and long-lasting release but limits the release of toxic quantities of Ag⁺ into any biological environment. The lack of toxicity was demonstrated in a cell culture experiment involving HEK293 (kidney) and T24 (bladder) cells (FIG. 11). This Figure demonstrates a lack of toxicity as compared to a control material. This phenomenon of precipitated and protective AgCl on the surfaces of compositions comprising silver nanoparticles, however the rate at which these compositions attain this level of protection is governed by the need for silver nanoparticles to oxidize to release Ag(+), the active antimicrobial form of silver. This rate is slow by comparison to Ag-SCER compositions as the silver is already in its needed and oxidized form. In addition, when silver nanoparticles oxidize, tissue toxic superoxide is formed thus indicating the Ag-SCER compositions are inherently superior in a number of ways. Generally, the surface area of the device is a significant factor in delivery (e.g. foams yield high surface areas, versus a lower surface area, textured or solid composite material). Surface area of different constructs can be controlled, for example by material choice, and by fabrication and molding techniques (such as spraying, coating, blowing, molding and extrusion techniques that include co-extrusion). In certain embodiments it is important to restrict contact of an activated (e.g., silicone) composite material with a surface (e.g., an inner lumen) or portion of a device the composite is being attached, layered or molded to. The hydrophilicity of the polymer matrix may also play a role in the surface release characteristics of materials and devices of the invention.

The dissociation constants of the fine particulate activated ion-exchange polymer salt particles can be compared to the counterpart simple salts, particularly for silver given the known (low) solubility for silver salts. For example, silver sulfate (Ag₂SO₄) possesses a solubility constant (Ksp) of 1.2×10⁻⁵, silver chloride (AgCl) possesses a Ksp of 1.77×10⁻¹⁰, and silver phosphate possesses a Ksp of 1.8×10⁻¹⁸. As such, strong cation exchange fine particulate activated ion-exchange polymer salt particles modified to include silver will certainly possess a Ksp<1.2×10⁻⁵. With the replacement of silver by a cationic replacement ion and its simultaneous release and pairing with an anion (chloride, phosphate etc.), dissociability of the product salt is important. A surprising advantage of the instant invention is that replacement of the departing ion (e.g., silver) from the fine particulate activated ion-exchange polymer salt particles remedies the general concern of void spaces that would otherwise form when soluble components dissolve from conventional polymers and coatings.

To control dissociability and/or drug delivery kinetics of biologically active ionic agents from activated polymer salt materials and related composites, more and less hydrophilic and hydrophobic polymer matrices can be used. Distinct performance characteristics provide for sensitive construction of activated polymer salts having a full range of activity modalities, from purely surface activity to increasing levels of reversible or dissociable loading (including adjustable release and solubilization of initially bound, biologically active counter-ion agent, as can optionally be triggered by contact with physiological/ionic fluids). For preparing materials that can provide different release profiles and activated polymer salt constructs having different activities and dissociation potential/kinetics, a wide range of useful ion-exchange polymer salts are provided.

In more detailed embodiments of the invention, the fine particulate activated ion-exchange polymer salt materials thus produced are useful in a wide variety of biomedical applications, compositions, materials, polymer composites, and devices including devices where a hydrophilic matrix (carrier) is employed. Such applications include hydrophilic coatings on the surfaces of medical devices such as catheters (tubing) and hydrophilic carriers such as in foams, sponges, biosensors, and sheet-stock materials that can be used in wound healing (vacuum-assisted closure), wound dressings including wound contact layers, vaginal sponges and hard plastic enclosures of medical equipment used within the hospital/clinical environment and the like.

Beyond medical devices, tubing plays a significant role in food handling and production, pharmaceutical preparations, and infection-susceptible and other medical devices. As such, tubing materials of the invention are useful in these and other diverse industries, to prevent or minimize bacterial, fungal and viral contamination without excessive leaching of undesired or toxic byproducts.

Tubing is incorporated within many medical devices including urinary catheters, ureteral stents, cerebral shunts, central venous catheters including peripherally inserted central catheters (PICC line), dialysis catheters, wound drainage catheters, endotracheal tubes, pacemaker and implantable cardioverter defibrillator lead bodies for example. Within these and related devices, an approach that is an alternative to making entire lead body from the composite, the lead connector bundle (i.e. the over-molded part of the lead where the distal and proximal electrode connectors are immobilized, for both pacing and defibrillator leads) can be formulated to include a micronized antimicrobial strong or weak cation exchange resin. Endotracheal tubes as used for airway management can be manufactured completely from PVC or silicone Ag-SCER composites, including the retention cuff, a balloon that is filled with air that sits at the branch leading to the lungs. The retention cuff is a problematic component because as subglottic fluid builds along and on top of the retention cuff, any seepage can result in ventilator-associated pneumonia (VAP). One solution involves disinfection of the subglottic fluid by the Ag-SCER composite it is in intimate contact with. In this application, the Cu(II)-SCER may also be employed. The composites possess good mechanical properties when compared to control materials. For example, 50 shore A silicone rubber demonstrated a tensile strength of 10.9 MPa and % elongation of 1820. After 60 days of soaking in artificial urine and the Ag-SCER silicone composite (aged 2-years) possessed tensile strength of 8.6 MPa and % elongation of 1660. Although the properties of the composite were slightly diminished by 21% (tensile strength) and 8.8% (% elongation) as compared to controls aged in identical fashion. For devices not requiring operation under tension, changes of this magnitude are acceptable. By reducing the percentage of the fine particulate activated ion-exchange polymer salt material or reducing the particle sizes below 1 micron in the composition, these properties can be improved upon.

In yet another embodiment, continuous positive airway pressure therapy (CPAP) uses a machine to help a person who has obstructive sleep apnea (OSA) breathe more easily during sleep. A CPAP machine increases air pressure in your throat so that your airway does not collapse when you breathe in. The majority of the CPAP masks in the market today are made from silicone, and a few are made from some gel materials. Almost all are latex free. All plastic components of the CPAP system can be substituted with antimicrobial materials of the invention.

Biofilm-induced catheter-associated urinary tract infections (CAUTIs) result in considerable morbidity while contributing to large increases in the cost of health care. CAUTIs are the most common of healthcare-associated infections (HAIs) costing >$600M/yr. to treat and resulting in ~13,000 deaths/yr. Ureteral stents are tubular devices designed to maintain an open ureter and enable the kidney to drain urine into the bladder. Stents are labeled for short duration use although they may remain in place for 3 mos, or longer. It is known that bacterial colonization of these devices, over time, results in urinary tract infection (UTI). The majority of UTIs in stented patients result from bacterial or fungal pathogens and the devices remaining in place for periods longer than 3 mos. Detection of bacteria in the urine of a catheterized person is referred to as catheter-associated bacteriuria (CAB) and is defined as the presence of at least 10e8 colony forming units (CFU)/L of one or more causative microorganisms with or without the symptoms of a urinary tract infection (UTI). The invention provides novel and surprisingly effective biomaterials for use in ureteral stents and urological catheters, which potently reduce bacterial adherence and bacterial counts in urine, colonization and infection to minimize UTI incidence and severity.

Ag-modified strong cation exchange resin (Ag-SCER) was milled to ~5-micron particle size and the powder incorporated into silicone rubber (0.5-10 wt %) and the material was easily extruded and molded into tan-colored tubing and slabs for test purposes. The material was coated with polyvinylpyrrolidone for lubricity and the tubing cut to lengths (~24 inches) and the materials were packaged into Tyvek sterilization bags and ETO sterilization carried out. The tubing and slabs were used to evaluate the effectiveness of the surface against a variety of bacteria in static and flow models using artificial urine. In 24-hour format the slab surface, as per the ASTM E2180 assay was shown to be highly effective at killing gram-negative and gram-positive organisms to >99.99% reductions (4-log 10). Routinely, bacterial reductions have been measured to be 100% and thus the log 10 reductions are determined as based on bacterial growth on the control surface. The composite material was observed to be equally effective with a 10-micron thick coating of polyvinylpyrrolidone on the surface thus indicating that the Ag+ can migrate through/across the coating to reach the outer surface. Polyvinylchloride and styrene-ethylene-butylene-styrene copolymer perform identically at equal loadings of Ag-SCER. Beyond the tubing component of the urinary catheter, the retention balloon, a balloon that is filled with sterile water and resides within the bladder at the point where the bladder drains into the catheter located within the urethra) can be fashioned from a silicone, PVC, TPE or polyurethane material compounded with an Ag-SCER additive for example. The balloon component has been shown to be durable and stable and offers protection of a component that otherwise goes unprotected as coatings are difficult to apply to the balloon, and if applied, coating failure with balloon expansion is likely. Today, retention balloons are essentially not capable of preventing bacterial growth.

Double J (pigtail) ureteral stents are tubular devices commonly used with a variety of urological procedures to drain the kidney and maintain ureteral patency. With the aid of an introducer, ureteral stents are inserted through the urethra across the bladder and into the ureter to allow the proximal end to reside in the kidney and the distal end to reside in the bladder. When the introducer is removed the ends coil into pigtails and act to stabilize the device ends, one in the kidney and the other in the bladder. The devices are generally approved for short duration (≤28 days) but in some instances they can remain placed for months. Although stents are commonly used in urological practice, infection and fouling/encrustation can obstruct flow. These infections are classified as catheter-associated urinary tract infections (CAUTIs) even though they are “stent-related”. Worth noting is that within 3-4 months following implantation, nearly 100% of stents removed from patients are colonized with bacteria. Importantly, stents are small in diameter as they need to be inserted through the ureter and into the kidney entrance. This small diameter makes it too difficult to add any antimicrobial coatings due to the restriction associated with inner and outer tube diameters.

In other words, there is too little surface area to add any significant amount of coating. It is the intention of the stent proposed herein to reduce infection(s) and fouling, and as such reduce the need for antibiotic therapy that plays a role in bacterial resistance. Generally, when infection occurs, stent removal with or without replacement is essential to successfully treat the patient. CAUTIs are the most common of the health care-associated infections (HAIs) in U.S. hospitals. UTIs are multistep processes generally involving migration of gut bacteria to the periurethral space and subsequent colonization, inflammation, biofilm formation, enhanced colonization and epithelial damage from bacterial toxins and proteases. When a catheter or stent is introduced, the bladder is compromised, thus, enhancing the immune response producing inflammation-facilitated fibrinogen production and uropathogens that express fibrinogen-binding protein can further facilitate bacterial attachment. Hence, a significant causative factor of CAUTIs is that the materials used to manufacture urological devices are susceptible to protein fouling and bacterial adhesion thus encouraging bacteriuria and/or infection. The ideal urological biomaterial has yet to be developed. Device overuse and extended use-time increases biofilm infection and subsequently, antibiotic overuse, which heightens concerns of AM resistance. A noteworthy side effect of ureteral stenting also includes patient experienced (flank) pain that can be debilitating. This pain has been attributed to inflammation (as well as other possible causative factors) that is associated with the presence of the device inContact with sensitive tissues. To address this side-effect while minimizing or eliminating infection, a lubricious surface coating that incorporates a powerful steroidal anti-inflammatory agent of low solubility such as dexamethasone can be added to the device. The powerful antimicrobial effect of an Ag-SCER silicone composite, for example, is potent enough to overcome any diminished immune response that may be induced by the corticosteroid. Other coatings that can be added may also include nonsteroidal anti-inflammatory agents such as ketoprofen or ketorolac. A stent that reduces CAUTIs alone, or that reduces CAUTIs and minimizes patient discomfort will reduce costs, cut antibiotic use, diminish resistance, and save lives. In an experiment to evaluate the potential impact of protein adsorption on the efficacy of the material, Ag-SCER modified silicone (2.0 wt %) was continuously extracted with PBS and at time points of 0, 7, 14, 21, 28-days, the efficacy of the material was determined following a 3-hour exposure to fetal bovine serum (FBS) (FIG. 5). Exposure to FBS did not impact the material efficacy.

A variety of bacterial species are known to colonize ureteral stents and subsequently form biofilms on the outer and inner surfaces of the devices. It is organisms that produce urease that facilitate the formation of (luminal and extraluminal) mineral-encrusted biofilms by its action on urea, degrading it to ammonia and carbon dioxide thus increasing the pH of the urinary tract. Higher pH leads to precipitation of Ca++ and Mg++phosphates. The most problematic biofilms are crystalline in nature, and these can induce trauma to the bladder, urethra, ureter, and kidney epithelium with device removal. Also, crystalline biofilm debris can induce stone formation and blockage of urine flow when these biofilms are disrupted. If blockages go undetected, patients can suffer pyelonephritis and septicemia.

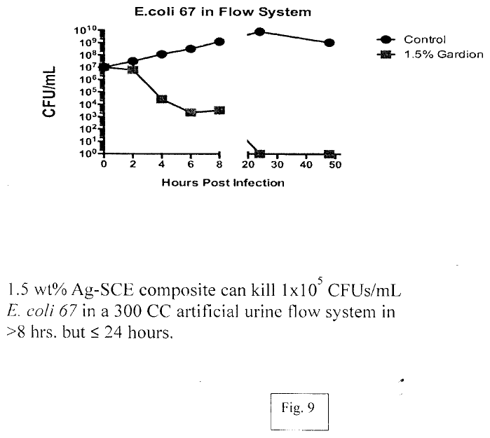

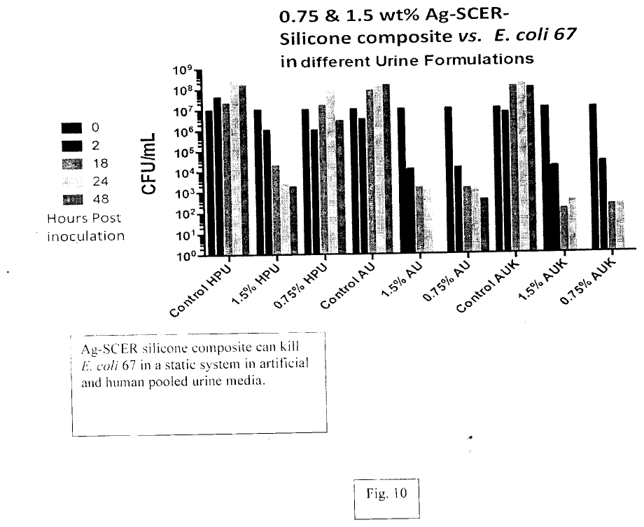

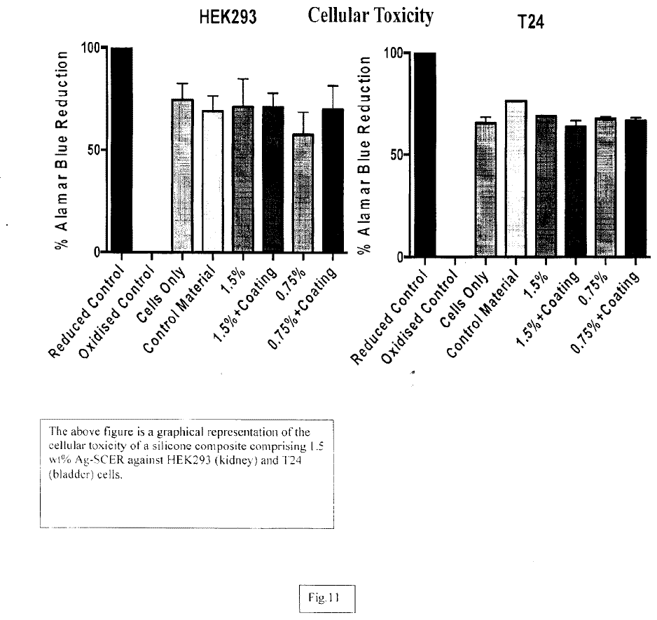

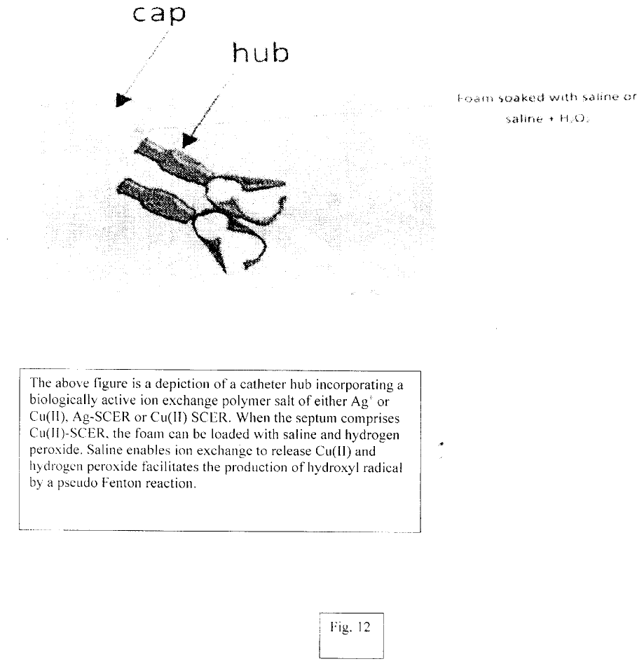

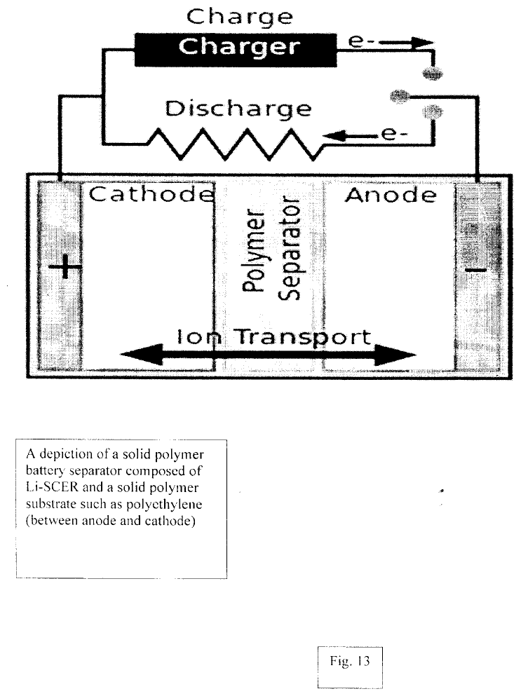

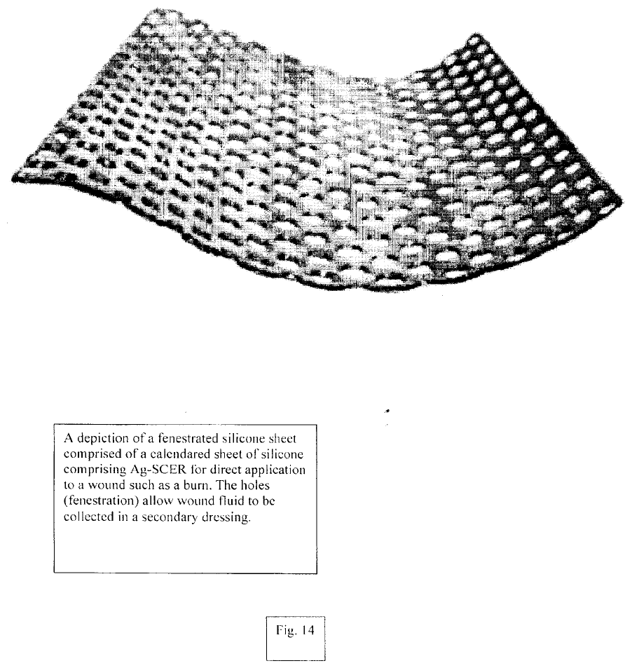

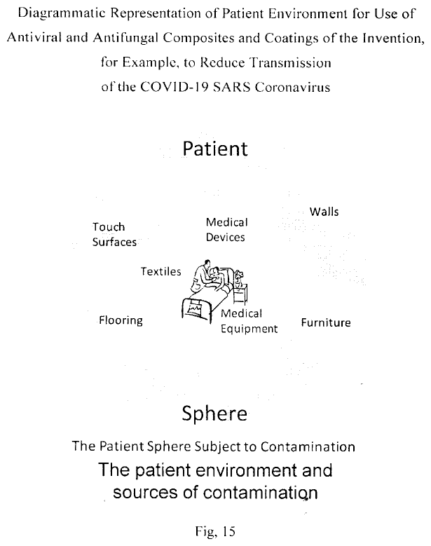

The use of Ag⁺-based coatings on devices and improved aseptic/sterility are the primary approaches to minimizing CAUTIs. Studies show that coated products only reduce the risk of CAUTI by 32% suggesting that Ag⁺ coatings may be beneficial only in acute care, i.e. for <2 weeks. Furthermore, antibiotic-impregnated catheters are beneficial in reducing CAUTI incidences for <7 days, highlighting concerns of using soluble drug-based coatings, particularly antibiotics. Moreover, infections are increasingly eliciting resistance, with 20% of all HAI pathogens reported to the National Healthcare Safety Network (NHSN) exhibiting multidrug-resistant phenotypes. These outcomes reinforce the need to develop biocompatible and AM materials capable of preventing bacterial invasion over an extended time-period, whereas a secondary and important benefit is the minimization of antibiotic therapy. In essence, the end of effective antibiotics is looming. Presently, about 17 million prescriptions of broad-spectrum antibiotics are administered in U.S. hospitals annually for the treatment of CAUTI. This is a significant risk to patients given the compounding contribution that antimicrobial therapy has to antimicrobial resistance. As such, a Foley catheter, or other medical device susceptible to infections that can be fabricated from a material resilient to infection/biofilm formation can greatly reduce the dependency on antimicrobial therapies for such device-related infections. Such devices can play a role in extending the useful of existence of current antimicrobial compounds. In an experiment designed to evaluate the antibacterial efficacy of silicone composites modified to include Ag-SCER (Ag-SCER) and a Ag-SCER/Cu(II)-SCER combination in comparison to Bard Lubrisil IC and the Dover silver-coated catheters (FIG. 2). This evaluation reveals that the novel catheter described herein significantly outperformed the commercially available products.

In another embodiment, a strong cation exchange resin is used to bind cysteamine, a known inhibitor of urease. Thiol compounds have been reported to be competitive inhibitors of urease. Those with a charged amino group in the beta position, such as cysteamine, are potent inhibitors whilst those that have a carboxyl group in the beta position, such as cysteine, are weak inhibitors. Cysteamine is readily bound to the acid forms of strong cation exchange resins (at full exchange capacity) to yield a cysteamine salt (a positively charged beta amino group. In the presence of urine, the cysteamine is released in its cationic form as the hydrochloride salt to readily inhibit the presence of urease expressed by uropathogens such as Proteus mirabilis.

In other medical device embodiments beyond catheters, implantable pulse generators such as pacemakers and defibrillators can be coated with an insulating substrate containing Ag-SCER, BA-SCER or Cu-SCER at concentrations ranging from 0.5 to 5.0 wt %. In yet another embodiment the pulse generator of a pacemaker or defibrillator is constricted with a header formed as a composite of Ag-SCER or other suitable additive such as a quaternary ammonium-SCER. FIG. 4 provides an overview of BA-SCER-silicone and BA-SCER/Ag-SCER silicone with comparison to Ag-SCER silicone compositions vs. E. coli. The BA-SCER composite alone is only partially efficacious against the gram negative organism.

In yet another alternative medical device embodiment, a biosensor, such as a glucose sensor, is dipped into a dispersion of a biocompatible outer (glucose limiting) membrane solution comprising approximately 1.0 wt % of Ag-SCER or Cu-SCER dispersed evenly throughout the membrane. It has become evident that transcutaneous sensors, such as Dexcom's G5 Continuous Glucose Monitoring sensor, are susceptible to inflammation related to bacterial fouling. The presence of these organisms and the resulting inflammation can lead to erroneous and unpredictable data. Alleviating the attachment of organisms and the subsequent formation of biofilms at the sensor surface can enable greater accuracy which is of importance given that therapy decisions can be made about the result of a sensor's reading. In other embodiments of the invention, the fine particulate activated ion-exchange polymer salt materials may be incorporated into bitumen, asphalt, or tar for the purposes of coating substrates. One such application may include coating of the inside of duct work in order to minimize pathogens in environments that require good adhesion and chemical stability for example. In addition, the incorporation of the fine particulate activated ion-exchange polymer salt materials into cellulose (paper) and/or gypsum board material can allow for the fabrication of gypsum wall board with antimicrobial properties as for example to minimize or prevent the growth of fungi. This may be done with the use of a copper salt of the fine particulate activated ion-exchange polymer salt material or a more active (organic) cationic fungicide derivative. One representative enabling embodiment of the invention involves the exposure of Stachybotrys chartarum (toxic black mold) to a scrub test panel painted with a water-based acrylic latex enamel paint modified to include 1.50 wt % of Cu(II) modified strong cation exchange resin. The resin contains 12 wt % Cu(II). In this study, an ASTM method (D3273) was employed, and inoculation and growth were compared to a control gypsum material. The paint completely eradicated and prevented the fungus from growing over a period of 4-weeks and the control became overrun with the fungus. Similarly, acoustic ceiling tiles fabricated from a cellulose-based substrate can be formulated prior to fabrication with a Cu(II) activated ion exchange resin, preferably of the strong cation exchange variety or the panel can be spray coated with a lacquer or acrylic latex enamel comprising a Cu(II)-SCER additive. Cu(II) is highly active against fungi. This is a remarkable finding particularly in light of our findings that the Cu(II) did not leach from the paint (coatings) at levels close to the safe drinking water limits for Cu(II) set forth by EPA (1.3 mg/L) with continuous extraction in tap water (as determined using certified water test strips), dilute bleach (5%) and industrial quaternary ammonium disinfectant (Lysol).

In another embodiment, the identical 1.50 wt % loaded paint was tested in a 2-hour activity assay against methicillin-resistant Staphylococcus aureus. The Cu-SCER logged a complete kill with a 3.78 log reduction against the organism. At 1.5 wt %, the amount of Cu(II) present by weight is 0.18 wt %. The 3.78 log reduction, to some extent, is also dictated by the number of total bacterial organisms counted on the “comparison” control panel as the total log reduction cannot exceed this reference number.

Certain embodiments of the invention employ fine particulate activate ion-exchange polymer salt materials absent a polymeric binder, or with only an aqueous-based carrier that can be employed in order to disperse the particulate materials. For example, the fine particulate activated ion-exchange polymer salt materials may be used in farming to deliver fungicides, nutrients, or insecticides for example. One such example is an azide derivative of an anionic exchange material was synthesized. Azide is oftentimes used in pest control. In this instance, the fine particulate activated ion-exchange polymer salt materials may be encapsulated into a starch carrier thus allowing for safer and more facile spreading of the particulates.

In other embodiments of the invention, azide derivatives of fine particulate activated ion-exchange polymer salt materials are employed in the fabrication of airbags and are safer to handle and will perform better than the conventional airbag material sodium azide. Similar products of the invention also possess preservative activity and can also be used in the fabrication of detonators and other explosives (particularly employing high surface area constructs). For these applications crosslinked materials are employed wherein the crosslinker is enzymatically degradable, for example a divinyl adipate. Similar to azide, fulminate derivatives of fine particulate activated ion-exchange polymer salt materials may also be employed as a detonator composition.

In certain embodiments of the invention, cyano (CN(−)) derivatives of fine particulate activated ion-exchange polymer salt materials are employed as a means of forming cyanide. These derivatives can be used as a means of dispersing/releasing cyanide.

Fine particulate activated ion-exchange polymer salt materials of the invention are also useful for environmental recovery of soluble metallic and organic contaminants, particularly in fresh water. These compositions and methods employ high surface area foam materials containing dispersed fine particulate activated ion-exchange polymer salt materials. The subject foams, pads and/or sponges can be constructed for capture of selected metal(s), for example lead (wherein Pb (II) is captured by a weak cation exchange material integrated in a moderately hydrophilic material coated onto a three-dimensional lightweight substrate such as a polymer foam, metal substrate such as a fence-like substrate, tubes with pores, or a carbon construct, for example). These constructs are placed into an environment at risk of contamination and removed and replaced as needed. In an alternative example, a solid, porous substrate comprising Cu(II)-SCER can be submerged within containers of water gathered in for transfer to locations where it can be consumed and/or used for cooking in order to eliminate contamination by microorganisms of other parasites.

In certain embodiments of the invention, fine particulate activated ion-exchange polymer salt materials are combined with other polymer materials to produce biologically active solidified polymer composites. The fine particulate ion-exchange polymer salt is generally admixed in effective amounts with precursors of a thermoset or thermoplastic or photocuring polymer, to form fluid or semi-solid antimicrobial polymer composite mixtures. The mixtures can be solidified using a wide range of polymer manufacturing methods and conditions and in a diverse array of composite mixtures and final hardened composite forms (e.g., solid cast or molded articles or components, extruded, spun into fiber, or blown into solid or cellular set polymer (film) materials, laminates, coatings, paints, and the like. The biologically active solid polymer composites are formed by solidifying or curing the polymer precursors admixed with the fine particulate biologically active ion-exchange polymer salt material. In some embodiments, the fine particulate polymer salt material is distributed throughout the resulting, activated polymer composite for example as in a polypropylene suture, or nonwoven material as fabricated by drawing, extrusion, or spinning and incorporating an evenly distributed composition of the fine particulate polymer salt material(s). In such an embodiment, the fine particulate polymer salt material may be modified to include one or more of tobramycin, minocycline, or silver or mixtures of the individual fine particulate polymer salt materials may be used for example in order to render the suture material antimicrobial. In another example embodiment, a polypropylene composite material, for example to include silver (I), copper (II), zinc (II), benzalkonium, sodium, alone or in combinations thereof can be spun into a nonwoven (fabric) composition and the nonwoven material composition used in the fabrication of air filters, carpet, furniture, medical textiles, and geotextiles. One example application is for use as a (diagnostic) substrate when formulated to include Na-SCER. Such a substrate can be placed below ground, allowed to dwell for some period of time and subsequently harvested (removed from the ground) and the fabric analyzed for metal uptake (e.g. Cu (II), Fe (II), As (III, V for example) with the aid of atomic absorption (AA) or inductively coupled plasma (ICP) spectroscopy. In yet another embodiment a hernia repair patch may be constructed using similar means yet with an IRP69 derivative functionalized with tobramycin for example. In other embodiments, the fine particulate polymer salt material is unevenly distributed within the final solid composite. This can be achieved, for example, by mixing the fine particulate activated polymer salt material only with specific parts or layers of a composite mixture prior to hardening. In this manner, setting of the ion-exchange polymer salt in the hardened polymer composite will determine its localization in a predetermined functional/spatial distribution within the hardened composite, for example by concentrating the polymer salt particles at upper, outer, luminal, or other defined sites, surfaces, layers or areas within a solid composite form or structure. Methods available for site-specific location of the particles includes coating these areas using dipping, spraying, or painting lacquers or latexes onto non-masked surfaces, site-specific molding over metal or polymer substrate, or co-extrusion that may include one or more of a line along the exterior or interior of a single lumen or multi-lumen tube for example, an outer surface or inner lumen of a tube. Because extrusion is a continuous process, such surfaces will be continuous along the construct in one form or another. If more than one modified fine particulate activated ion-exchange polymer salt material is desired in a single construct, more than one feed may be used.

In another exemplary embodiment, PVC or PEVA mattress protectors can be readily fabricated from PVC or PEVA film incorporating Ag-SCER or Cu-SCER for example. The presence of an antimicrobial agent additive can prevent microorganisms particularly those from urine, but also from feces, from leading to a bacterial, viral or fungal soiling which can create odors within or on the mattress.

In yet another example, inkjet technology may be employed to deposit an array of various lacquers comprising one or more of a fine particulate activated ion-exchange polymer salt material onto the surface of a medical device, diagnostic device, or packaging material. In one other such example, small molecule probes may be isolated onto particles and further isolated onto an array to probe for viruses, bacteria, or perhaps the genetic makeup of a parasite or other infectious disease variable.

The thermoset or thermoplastic or photocuring polymers used to form solid biologically active polymer composites herein can be selected from a broad array of useful polymers, for example polysiloxane, polyalkylene, polyamide, epoxy, polycarbonate, polyester, vinyl, acrylic, and polyurethane polymers, and combinations thereof. In certain embodiments, the thermoset or thermoplastic or photocuring polymer mixed with the fine particulate activated ion-exchange polymer salt material (comprising the “polymer composite mixture”) is cast, sprayed, formed, spun, blown or extruded into a desired shape or article prior to solidifying. The polymer composite mixture may be solidified by any means generally used, for example by drying or curing under normal conditions (e.g., at room temperature in air). In certain embodiments the polymer composite mixture may be cooled during hardening process, while in other embodiments the polymer composite mixture is cured using heat. In additional embodiments, the second, thermoset or thermoplastic or photocuring polymer precursors are provided in the form of a polymer lacquer, the lacquer comprising a solvent, the solidifying step comprising evaporating the solvent from the polymer lacquer to form the solid biologically active polymer composite. The resulting solid biologically active composite may contain a selected amount or weight ratio of the activated ion-exchange polymer salt material, as described, to optimize the composites for specific uses and concentrations (or effective dosage levels) of incorporated biologically active ionic agent to mediate specific biological activities and/or therapeutic effects. Other means of curing/processing discoloration include oxidation or chemical conversion of the resulting discolorizing species by exposure to oxidizing agents such as hydrogen peroxide or halides such as aqueous iodine for example.

In certain embodiments relating to formulation of polymer composites, the thermoset or thermoplastic or photocuring polymer precursors are non-vulcanized silicone rubber precursors. These precursors combine to form a highly adhesive silicone gel or liquid. The silicone gel or liquid is cured after addition of a selected amount or ratio of the fine particulate activated ion-exchange polymer salt, often at an elevated temperature of about 150° C. (typically for a curing period of about 5 to 10 minutes). In certain embodiments where the fine particulate ion-exchange polymer salt incorporates an oligodynamic metal, such as silver, as the activating ionic agent, curing of the silicone polymer results in discoloration, marked by darkening (often with a reddish tint) of the hardened biologically active polymer composite.

Yet another useful and unexpected discovery of the invention is that certain activated polymer composites may be further processed to reverse normal curing discoloration, to yield a re-lightened final solid polymer composite. The further processed, lightened polymer composite is more advantageous for medical and other uses, from both a basic cosmetic appeal perspective (lighter polymer materials appear more hygienic), and from an actual hygiene and safety perspective (because the lighter color allows for better visualization of soiling agents and contaminants, including possible toxic, pathogenic or corrosive contaminants).

Reversal of discoloration from normal curing of activated polymer composites of the invention (particularly those containing silver and other metallic ions) can be achieved by employing the novel polymer composite mixtures provided herein, and by subjecting these discrete polymer composite mixtures to a modified curing regimen or treatment of the composition using an oxidizing agent such as hydrogen peroxide or iodine solution for example. The discovery includes the use of extended curing times and/or elevated curing temperatures, which alone or in combination (typically in the presence of oxygen) yields a surprising reversal of color darkening observed following conventional curing procedures. In another embodiment, the composition is exposed to aqueous iodine, bromine, or chlorine or hydrogen peroxide for example.

Discoloration reversal can be achieved for example by extending curing times beyond conventional curing times (e.g., 5-10 minutes for silicone). Thus, in certain embodiments curing times may be extended for an additional 10-30 minutes, one-three hours, or longer depending upon composition of the polymer composite. In other embodiments initial and/or extended curing may be conducted at a higher temperature than conventional curing, for example at temperatures greater than 150° C., greater than 175° C., up to 200° C. or higher. In exemplary protocols, normal curing is conducted at 150 degrees for 5-10 minutes, and extended curing is carried out for an additional time period until a desired extent of discoloration reversal is observed. These curing changes, in various protocols following the teachings herein yield novel biologically active polymer composites having desirable, lightened color properties for medical and other uses.

Certain activated polymer composites of the invention are made using multiple different polymer precursors, for example a mixture of polymer precursors of polyalkylene, polysiloxane, polyamide, epoxy, polycarbonate, polyester, polyol, polyarylene, vinyl polymer, acrylic polymer (polyacrylonitrile, polyacrylate), asphalt, bitumen, polysaccharide, cellulosic, and/or polyurethane. The polymer precursors for making the activated polymer composites can include one, two or more types of precursors selected from silicone rubber, methacrylic acid, polypropylene oxide, polyethylene oxide, polyvinyl alcohol, polyurethane, hydrocolloid, a polyester, a polycarbonate, a vinyl polymer (PVC, PVA, PVAc, Polyvinylidene chloride, polyisoprene, styrenic polymers including polystyrene, styrene-isobutylene-styrene triblock copolymer (SIBS), acrylonitrile-butadiene-styrene copolymer ABS, styrene-butadiene-styrene copolymer (SBS), hydrogenated vinyl polymers including hydrogenated SBS, e.g. styrene-ethylene-butylene-styrene copolymer (SEBS), and polyalkylenes such as polyethylene and polypropylene, a polyamide, an epoxy, a phenolic resin, a polyurea, an acrylic, a cellulosic, a fluoropolymer, or a biopolymer such as collagen, hyaluronic acid, gelatin, a hydrogel polymer, and/or an alginate, among other polymer types. The precursors may include like or different monomers including monomers of block, graft and statistical copolymers, asphalt, bitumen, and/or blends of various polymers.

Solid polymer composites of the invention can include a plurality of polymer chains from at least one polymer type forming a solid polymer matrix. The same polymer precursors can be used to form different types of solid or semi-solid polymer matrices. For example, a silicone rubber polymer solid or semi-solid matrix can comprise a room temperature vulcanizing (RTV) silicone rubber adhesive, a tacky silicone gel, a liquid silicone rubber, or a high consistency silicone rubber. The solid polymer matrix may be an elastomer, which when in solid form employed for making durable materials and products will often have a hardness (durometer) in the range of 10 shore A to 90 shore D. In some embodiments, the hardness of the biologically active solid polymer composite material and constructs may be between 15 shore A and about 65 shore D. Other “engineering polymers” may also be employed. These include acrylics, polycarbonate, poly(ether-ether-ketone) (PEEK), acrylonitrile-butadiene-styrene (ABS) polymers, as well as other materials amenable to thermal processing or processing into lacquers for coating processes.

Production of biologically active solid polymer composites of the invention is schematically depicted in manufacturing Scheme 4, where R is a group containing carbon and n is greater than 1.

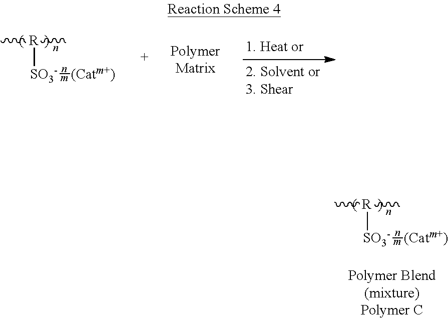

The polymer matrix (precursors) may be a polymeric composition that includes one or more useful polymer precursor types, for example from the group silicone rubber, polyurethane, a polyester, a polycarbonate, a vinyl polymer (PVC, PVA, PVAc, Polyvinylidene chloride, polyisoprene, SIBS, ABS, SBS, polystyrene, hydrogenated vinyl polymers, e.g. SEBS), a polyalkylene such as polyethylene, a polyamide, an epoxy, an acrylic, a cellulosic, a fluoropolymer, or a biopolymer such as collagen, hyaluronic acid, gelatin, a hydrogel polymer, and/or an alginate.

In some embodiments, the polymer precursors comprising the polymer matrix may be provided as one or more polymer precursors in a substantially unsolidified (fluid or semi-solid) state. Prior to solidifying the polymer composite, the precursors are blended with biologically active ion-exchange polymer salt particles to form a polymer composite mixture. This mixture is then solidified to form activated solid polymeric composites, and related biomaterials and products. 190] In various embodiments, activated polymer composites are made with any of a diverse array of polymer precursors classified as thermoplastic, thermoset, elastomer, and/or rigid polymer precursors. Exemplary polymeric precursors include, but are not limited to, one or more of polyalkylene, polysiloxane, polyamide, epoxy, polycarbonate, polyester, polyol, polyarylene, vinyl polymer, acrylic polymer (polyacrylonitrile, polyacrylate, polymethylmethacrylate), asphalt, bitumen, polysaccharide, cellulosic, and/or polyurethane. Exemplary polymer precursors comprise nonvulcanized silicone rubber precursors.

In other embodiments, solid (hard materials) such as polycarbonates, and epoxies can be combined with fine particulate biologically active polymer salts and these types of polymer composite mixtures can be formed and solidified to provide harder materials having smoother, harder, more impact resistant and defect-free surfaces than other polymer composites. Exemplary biologically active polymer composite materials produced according to the teachings herein are listed in Tables 2a-c below, for illustrative purposes.

TABLE 2A

| | Matrix | Active Agent-Singular formulations | | | | | | | | | | | | | |
|---|---|---|---|---|---|---|---|---|---|---|---|---|---|---|---|
| RESIN | Material | Ag | BA | CP | Dox | Rif | Min | CHX | Oct | Zn | Fe(II) | Cu(II) | Ag/Cu | Ag/Zn | BA/Ag |
| SCER | Silicone | X | X | X | X | X | X | X | X | X | X | X | X | X | X |
| | Poly- | X | X | X | X | X | X | X | X | X | X | X | X | X | X |
| | urethane | | | | | | | | | | | | | | |
| | Epoxy | X | X | X | X | X | X | X | X | X | X | X | X | X | X |
| | Acrylic | X | X | X | X | X | X | X | X | X | X | X | X | X | X |
| | Poly- | | | | | | | | | | | | | | |
| | ethylene | | | | | | | | | | | | | | |
| | Poly- | X | | | | | | | | | | | | | |
| | aspartate | | | | | | | | | | | | | | |
| | Polyvinyl | X | X | | | | | | | | | X | | | |
| | chloride | | | | | | | | | | | | | | |
| WECR | Silicone | X | X | X | X | X | X | X | X | X | X | X | X | X | X |
| | Polyuret | X | X | X | X | X | X | X | X | X | X | X | X | X | X |
| | hane | | | | | | | | | | | | | | |
| | Epoxy | X | X | X | X | X | X | X | X | X | X | X | X | X | X |
| | Acrylic | X | X | X | X | X | X | X | X | X | X | X | X | X | X |

TABLE 2B

| | | Binary Formulations (Individual Singular Additives) | | | | | | |
|---|---|---|---|---|---|---|---|---|
| | Matrix | Ag & | Ag & | Ag & | Ag & | Rif & | Dox & | Ag & |
| RESIN | Material | BA | CP | Zn | Zn | Min | Rif | CHX |
| SCER | Silicone | X | X | X | X | X | X | X |
| | Poly- | X | X | X | X | X | X | X |
| | urethane | | | | | | | |
| | Epoxy | X | X | X | X | X | X | X |
| | Acrylic | X | X | X | X | X | X | X |
| WCER | Silicone | X | X | X | X | X | X | X |
| | Poly- | X | X | X | X | X | X | X |
| | urethane | | | | | | | |
| | Epoxy | X | X | X | X | X | X | X |
| | Acrylic | X | X | X | X | X | X | X |

TABLE 2C

| | | | | | Product |
|---|---|---|---|---|---|
| Active Agents | | | Polymer | Company | Name |
| Ag = Silver | Rif = Rifampicin | Zn = Zinc | Silicone | Nusil | 4950 |
| BA = Benzalkonium | Min = | Fe = Iron | | Dow | Q7- |
| | Minocycline | | | Corning | 4750HCR |
| CP = Cetylpyridinium | CHX = | Cu = Copper | Polyurethane | Lubrizol | MG-8020 |
| | Chlorhexidine | | | | |
| Dox = doxycycline | Oct = Octenidine | | | Lubrizol | TG-500 |
| | | | | Lubrizol | SP-80A- |
| | | | | | 150 |

| | | | |
|---|---|---|---|
| Ion Exchange Resins | Epoxy | EPO- | 301 |
| | | TECH | |
| WCER = Poly(methacrylic acid-co-divinylbenzene) | Acrylic | SCIGR | 40 |
| | | IP | |
| SCER = Sulfonated Poly(styrene-co-divinylbenzene) | | | |

Primary products of the invention (biologically active ion-exchange polymer salts) can be combined with a variety of thermoset or thermoplastic or photocuring polymer precursors to make solid composites having a range of biological surface activities (and optionally, non-surface drug delivery activity) and a commensurate array of applications and methods of use. Surface activation of the inventive polymer composites (i.e., specific biological activity or activity potential, exhibited by an exposed surface of the polymer composite) can vary depending on the type and identity of the biologically active ionic agent (bound to) incorporated into the ion-exchange polymer salt, as well as on the amount and distribution of the active polymer salt within the hardened polymer composite.

Among the powerful discoveries here, biologically active solid polymer composites can incorporate varying amounts of the activated ion-exchange polymer salt material to yield predetermined or “metered” activity potential at the solid polymer composite surface. Varying the amount or distribution of activated ion-exchange polymer salt can increase or decrease the surface activity of the finished polymer composite, by increasing or decreasing a surface concentration (e.g., by weight or by surface area) and activity of the biologically active ionic agent associated within the activated polymer salt. This ability to adjust or “meter” surface activity of polymer composites is readily achieved according to multiple teachings herein. In one example, this is achieved by adjusting “loading” of the ion-exchange polymer as described (e.g., by increasing or decreasing a percentage of biologically active counter-ion exchange for initial counter-ion within the organic ion exchange polymer-expressed for example as a percent of actual exchange (with activating counter-ion) of real or theoretical maximum ion-exchange potential, or in another example as, e.g., weight of silver or other active counter-ion loaded per total dry weight of ion-exchange material), such as described by wt % of biologically active ion. In another (alternative or complementary) method for controlling surface biological activity of activated polymer composites of the invention, the instant disclosure provides for variable or metered “dosing” of polymer composites by combining different amounts of fine particulate, biologically active ion-exchange polymer with thermoset or thermoplastic or photocuring precursors to form the activated composites. Surface activity potential (and in related embodiments dissociation and drug delivery kinetics) are therefore adjustable across a wide range of selectable values, simply by adjusting a weight percentage of activated polymer salt to thermoset or thermoplastic or photocuring polymer precursors, as described. A selected weight ratio of 10-20% of activated, fine particulate polymer salt combined with silicone precursors to form an activated composite, for example, will yield approximately twice the surface activity potential (and optionally twice the dissociation or drug delivery kinetic value) of a like composite formed using only 5-10% by weight of the activated fine particulate polymer salt.

In certain embodiments of the invention, the biologically active polymer salts and polymer composites are useful to prevent attachment, colonization and/or survival of microbes (e.g., bacteria, fungi, archaea, plants, or protozoa) or other pathogens (e.g. viruses) or parasites transmissible by surface contamination on a fomite or other targeted surface. To reduce or prevent secondary transmission of viable pathogens to a vulnerable living subject.

These distinctly potent antimicrobial (antibacterial and antifungal) and antiviral actions are readily demonstrated using conventional assays. For example, antimicrobially active polymer composites of the invention incorporating an ionic antibacterial agent (e.g., silver, or an ionic antibiotic) exhibit greatly reduced contamination compared to the same material control active. In side-by-side tests (e.g., of the same silicone polymer, with and without incorporation of an active polymer resin salt resin as described here, where the test and control polymers are subject to the same inoculum of contaminating test bacteria) using standard assays (e.g. ISO 22196 of ASTM E2180), re-plating the contaminating bacteria (e.g., by swiping an equal area of test and control, “contaminated” surface across an agar culture medium) to a secondary test surface demonstrates great efficacy of the materials of the invention in preventing and controlling microbial contamination. Comparable efficacy is obtained using related embodiments of the invention incorporating fungicidal and fungistatic ionic agents, antiviral ionic agents, and anti-parasitic ionic agents (while some of these agents will have efficacy against multiple pathogen groups) using standardized assays such as antiviral assays (e.g. ISO 21702-19, Measurement of antiviral activity on plastics and other non-porous substrates).

To quantify these distinct surface properties, the invention as tested using antimicrobially active polymer composites effectively prevents or reduces microbial contamination and transfer up to 100% in side-by-side assays (e.g., as demonstrated by Kirby-Bauer disk diffusion assays described below). In more detailed aspects, the biologically active polymer salts and polymer composites of the invention prevent or reduce persistent microbial contamination (and, commensurately reduce microbial transfer potential) by at least 20-30%, 30-50%, 50-75%, or 75-90%, up to as much as 90-95%, or 98% or greater compared to persistent contamination and transfer potential observed using control materials. In various assays demonstrating these novel activities, microbial survival, and virus survival/viability/activity and/or growth potential is significantly reduced within these value ranges after inoculating test and control surfaces, waiting for a suitable post-inoculation period (to allow for activity potential of the test and control samples to be expressed, e.g., to permit bactericidal and bacteriostatic activity to take place), followed by “transfer plating” or “transfer culturing” or subsequent infection of an appropriate cell line to test survival and viability/transferability of microbial or viral contaminants from the test and control materials/surfaces. The antimicrobial/antiviral determination is made, for example, by directly contacting contaminated test and control surfaces to a “transfer” culture plate or liquid culture medium, or using lavage to transfer any intact and/or viable microorganisms or viruses from test and control surfaces, then detecting presence, numbers, or viable contagious units (e.g., colony forming units, or CFUs or virions) in the transfer growth plate or medium or cell population. In one example of an antiviral assay (a plaque or cytopathic effect assay), the amount of viable virus remaining on a surface after a period of exposure can be determined by evaluating cell death (infection) following exposure of a surface extract to a cell line post exposure such as described in the ISO 21702-19 assay. According to these methods, the active polymer salts and composites of the invention exhibit extraordinarily high levels of surface decontamination activity (e.g., bactericidal and/or bacteriostatic surface activity). This potent activity manifests within as little as 1-10 minutes after inoculation/contamination of these unique biomaterials. Within a half hour after surface contamination, or in some instances after from one hour to three hours, full expression of maximal surface decontamination activity is observed for many antimicrobially active polymer salts and polymer composites of the invention. In many instances this amounts to an effective total surface decontamination, where consistently no viable microorganisms remain viable or transferable from a contaminated surface after a post-inoculation activity expression period. These observed results are truly remarkable in comparison to contamination and transfer data observed from similarly treated control biomaterials (i.e., comparable ion-exchange polymer salt materials not activated by association with biologically active counter-ions, or comparable polymer composites incorporating ion-exchange polymer salt materials not activated with biologically active counter-ion).

In exemplary embodiments, microbial survival and/or transfer potential (e.g., expressed in terms of microbial numbers or growth observed after transfer plating from the contaminated surface/material) from contaminated test samples (of either the fine particulate ion-exchange polymer salt, or polymer composites made therewith) is less than 50% of microbial survival and/or transfer potential observed from control samples. In other embodiments, the microbial survival and/or transfer potential for test materials is less than 25%, 15%, 5% or 1% of the microbial survival and/or transfer potential observed from control materials. These and even higher levels of decontamination and transfer risk reduction are achieved for various microbial pathogens, including different forms of pathogenic bacteria, as well as pathogenic fungi and other microbial pathogens. In exemplary, antibacterial materials and composites, the level of bacterial control and decontamination mediated by polymer salts and composites of the invention confers at least a 50-75% reduction, often a 75%-95% reduction, up to a 95%-100% reduction and/or prevention of persistent contamination and/or transfer risk. Also, the survival of microorganisms or virus particles as expressed by log reduction where 2-logs=99% and 4-logs=99.99% for example.

Results for post-contamination transfer potential, or infection risk, are even more surprising and beneficial using the antimicrobially active materials and composites of the invention. The subject materials and composites have such novel and powerful surface antimicrobial efficacy, they can substantially eliminate surface-to-living subject transfer of viable pathogens targeted by their surface-loaded ionic antimicrobial agents. For ease of description, retransmission potential (e.g., as measured by ability to transfer viable colony forming units of a targeted bacterium from a contaminated surface following a “decontamination period” (e.g., 10-30 minutes, 1-3 hours, or longer) is reduced by at least 75-95%, often greater than 95%, and reproducibly at levels of up 98-100% compared to similarly contaminated controls of like polymer materials not antimicrobially activated according to the invention.

The profound antimicrobial surface activity exhibited by novel polymer composites of the invention renders these materials widely effective against a large host of the most serious bacterial contaminants found in institutional care settings and environments. Effective materials and products are provided against the most refractory, costly and dangerous sources of infection found in medical and veterinary care hospitals, assisted living facilities, penal housing institutions, food processing and packaging facilities, and HVAC and other environmental control systems, among other environments. Targeted microbes subject to reduction of surface contamination, and elimination of surface-to-live subject transfer risk, as described, include, for example, Staphylococcus, Pseudomonas, Escherichia coli, Klebsiella pneumoniae, Legionella pneumophila, Mycobacteria, Streptococcus, Acinetobacter, Hemophilus, and Enterococcus, Aspergillus, and Listeria. Target viruses include enveloped viruses such as, but not limited to influenza virus and coronaviruses such as SARS-COV-2, and non-enveloped viruses such as norovirus.

Yet additional advantages afforded by the instant invention include a novel utility and efficacy against infectious targets resistant to many drugs, such as MRSA (methicillin-resistant Staphylococcus aureus), resistant Streptococcus strains, and resistant airborne pathogens such as Mycobacterium tuberculosis and Legionella pneumophila. Using the embodiments of the invention described herein, resistant organisms may be addressed using compositions of distinctly modified fine particulate active ion-exchange polymer salt materials in combination. In one embodiment a silver-modified fine particulate active ion-exchange polymer salt material is combined with a chlorhexidine-modified a fine particulate active ion-exchange polymer salt material and the mixture is added to a polymer composition to produce a binary delivery system. Because each of the antiseptics kill bacteria using unique mechanisms, the likelihood of selecting for resistant strains is greatly diminished.

Yet additional advantages afforded by the instant invention include a novel utility and efficacy against infectious fungal diseases such as onychomycosis (fungal infection of the toe- and fingernails), tinea pedis, jock itch, ring worm, or cutaneous candidiasis. Antifungal agents can include copper (II), polyenes, imidazoles, triazoles, thiazoles, allylamines, echinocandins (caspofungin), flucytosine, and crystal violet. Generally, the aforementioned functional compound types may be used topically more effectively than by oral delivery. For example, for the treatment of onychomycosis, where Trichophyton rubrum is the most common dermatophyte involved in onychomycosis. Other dermatophytes that may be involved are Trichophyton interdigitale, Epidermophyton floccosum, T. violaceum, Microsporum gypseum, Trichophyton tonsurans, and Trichophyton soudanense. Topical agents include: clotrimazole, amorolfine or butenafine nail paints. All of these compounds are amenable to incorporation into the fine particulate organic ion exchange materials. Topical treatments need to be applied daily for prolonged periods (at least 1 year). For example, terbinafine-modified fine particulate active ion-exchange polymer salt material may be a candidate for treatment. Incorporation of this salt into a hydrophilic lacquer to be spread onto the nail bed is anticipated to be an appropriate treatment. In another embodiment reflective of the flexibility to mix and match various active organic ion exchange species, a laboratory bench to be used for tissue culture for example may be fabricated to include a mixture of antibacterial, antifungal, and antiviral agents thus minimizing the likelihood of contamination of cell lines from environmental contamination.

Antiviral compounds such as acyclovir (a synthetic nucleoside for treating herpes zoster and genital herpes), zidovudine or azidothymidine (a nucleoside analog for treating HIV/AIDS), abacavir (a nucleotide reverse transcriptase inhibitor), and lamivudine (a nucleoside nucleotide reverse transcriptase inhibitor) are readily bound to the fine particulate active ion-exchange polymer to yield the organic ion exchange salt. One potential application for the antiviral-modified fine particulate active ion-exchange polymer salts is to include the particulate into a hydrophilic matrix for placement into the vagina or anus for the delivery of the drug over time. Both locations are ideal for drug delivery due to the high vascularity thus allowing the drug to be effectively administered. The antiviral efficacy of composites of Cu(II) SCER-modified materials can also be exploited. Cu(II) is the active species that provides the ability of copper and copper alloy surfaces against to deliver approximately 1-log reductions against viruses. Dissolved Cu(I) and Cu(II) on these metal surfaces, resulting from metal oxidation, is active against enveloped viruses and it has been indicated to be active against non-enveloped viruses as well. Cu(II)-SCER composite coatings (e.g. acrylic latex enamel (ALE) paint) is highly active against coronavirus 229E, an enveloped surrogate virus for SARS-COV-2. It has been shown that a 2.0 wt % Cu(II)-SCER-modified ALE (Glidden, PPG), delivered a 99.7% log reduction in 2 hours and >99.9% in 24-hours. The 24-hour result was limited given that the control comparison panel (recovered virus) was not large enough to provide for a greater reduction. The noted antibacterial and antifungal activity of Cu-SCER composites is likely the result of the oligodynamic metal species binding to proteins (at sulfhydryl) and producing ROS and/or altering cell respiration. It is likely that on viral surfaces the effect is much the same, i.e. Cu(II) binding to protein to inactivate the virus.

Other candidate active agents for the treatment of parasitic diseases can be incorporated onto the organic ion exchange backbones. For example, chloroquinone, hydroxychloroquine, mefloquine, or doxycycline for the treatment of malaria can be readily bound to SCERs and WCERs, as well as phosphates such as cellulose phosphate. Compounds for the treatment of amoebozoa infections that cause dysentery including azoles (metronidazole and tinidazole), diiodohydroxyquinoline, and paromomycin for example can be employed with IRP69, IRP64, or polyphosphates. Helminth (nematode) infection particularly of the intestinal tract in humans and livestock can be treated using SCERs and WCERs such as IRP69-, IRP64- or polyphosphate-organic ion exchange materials modified to include piperazine, benzamidazoles, levamisole, pyrantel, or morantel. These compositions may be incorporated into materials that may be used as worming agents

In an exemplary embodiment, a water filtration device fabricated from a nonwoven fabric (e.g. polyester) filtration units formulated to include one or more of the antimicrobial additives of the present invention may be used in the sanitation of water.

Yet additional advantages afforded by the instant invention include the ability to yield antiparasitic-modified fine particulate active ion-exchange polymer salt materials in order to provide novel utility and efficacy against infectious parasitic diseases that include treatment of sleeping sickness caused by Trypanosoma brucei) using Melarsoprol-modified material, sleeping sickness using Eflornithine modified material, vaginitis caused by Trichomonas using Metronidazole-modified material, intestinal infections caused by Giardia using Tinidazole-modified material, the treatment of visceral and cutaneous leishmaniasis using Miltefosine-modified material.

The novel biologically active polymer salts and polymer composites of the invention remain fully biologically active during preparation and for an extended period of shelf life thereafter, even though preparation of the polymer salts in fine particulate form involves non-solvent exposure and temperatures elevated to 85° C. or higher, and despite that curing of the polymer composites often involves elevated temperatures of up to 150 degrees, or 200° C. or higher. In addition, the biologically active polymer salts and polymer composites remain active with the biologically active ionic agent incorporated therein being stable to degradation, oxidation, chemical decomposition, and photodegradation for an extended shelf period after production as described. Additionally, the novel biologically active polymer composites of the invention retain not only their biological activity potential, but also their structural integrity for extended shelf and use periods. This activity retention and structural stability is marked by no greater than about 2 to about 5% of chemical loss, degradation, decomposition, destructive hydrolysis or oxidation for the biologically active ionic agents incorporated in the polymer salts and composites, and no greater than about 2 to about 5%, loss of tensile strength, environmental stress cracking, hardness change, or loss of elasticity of the composites during production, including during extended curing of composites at 200° C. with the exception of fine particulate organic ion exchange powder salt materials that may interfere with cure for example as a consequence of interference with a catalyst for example. In other embodiments, the stable retention of biological activity structural integrity of these novel polymer composites fabricated as compatible blends, i.e. the fine particulate organic ion exchange powder salt material does not interfere with curing of polymer systems or used as matrix materials, is marked by no greater than about 1 to about 5 wt % loss under reasonable operating conditions and when tested alone, the resin systems exhibit remarkable stability well beyond the stability measured using the simple ion salt counterparts of the biologically active component. In general, the fine particulate organic ion exchange powder salt materials possess overall greater chemical stability, reduced thermal degradation and decomposition, and greater stability to destructive hydrolysis or oxidation for the biologically active ionic agents incorporated in the polymer salts and composites, and no greater than about 5 to about 15%, loss of tensile strength, change in hardness and/or modulus, or loss of elasticity for the composites over 1-3 months, 6 months, and up to a year or more in normal storage conditions (e.g., at standard laboratory room temperature and humidity, without use or mechanical wear). In more detailed embodiments, activity retention and structural stability is marked by no greater than about 1 to about 20% of chemical loss, degradation, decomposition, destructive hydrolysis or oxidation for the biologically active ionic agents incorporated in the polymer salts and composites, and no greater than about 1 to about 20%, loss of tensile strength, environmental stress cracking, hardness change, or loss of elasticity for the composites following extended exposure (up to 1-3 hours or longer) of the cured or hardened composites to extreme temperatures exceeding 200 degrees, 300 degrees and even 400° C. (allowing for a much broader array of clinical and industrial uses and post-production treatments of these novel composites and biomaterials).

After periods of use, the surfaces of biologically active composites and related biomaterials of the invention may start to lose their peak biological activity potential. For example, the biologically active ionic agents incorporated in the composites may become partially exhausted due to mechanical abrasion and other mechanisms of loss, ionic dissociation (particularly when used in contact with physiological or other ionic fluids), chemical reaction, chemical change by oxidation or hydrolysis, photodegradation, or other types of removing, discharging, destructive, transforming or deactivating factors.

Among the most surprising and medically advantageous discoveries of the invention herein are materials having a surface biological activity that is “self-recharging”, “self-regenerating”, or “renewable” following an initial period of use (wherein an initial biological activity potential is partially or completely exhausted or discharged). In exemplary embodiments, a fine particulate biologically active ion-exchange resin material is integrated throughout a solid polymer structure to provide for renewable surface activation following discharge (e.g., due to surface wear or erosion, chemical or ultraviolet degradation of biologically active agents, release or dissociation of active ion-exchange resin material and/or biologically active ionic agents from the polymer surface, etc.)

In alternative embodiments, the biologically active ion-exchange resin material is integrated within an outer or inner surface portion only of a solid polymer configuration or a composition of materials such as with a coextrusion and may be absent from all or part of deeper internal, core or interstitial portions of the polymer structure. In other alternative embodiments, the biologically active ion-exchange resin material is integrated within a coating or multi-layer laminate formed of the solid polymer, which can be applied or co-formed to cover a different polymer or non-polymer structure that does not incorporate the biologically active ion-exchange resin material. In a representative embodiment, an adhesive film is formulated to include a strong cation exchange resin modified to include Cu(II) or Ag(I) or a quaternary ammonium ion and the adhesive films used to modify touch surfaces in locations where disease transmission by fomites might be anticipated to be significant. Within these and related embodiments, as the biologically active ion-exchange resin material, and or the integrated ionic biologically active agent, is discharged, degraded, dissociated or exhausted at the surface of the active polymer composite (e.g., by mechanical wear or debridement, light or chemical degradation, chemical reaction on contact with external chemical species, oxidation, hydrolysis, decomposition, and/or ionic dissociation of the active ionic agent through exposure to physiological or other ionic fluids, chemical reaction), most or substantially all of an original surface biological activity of the polymer structure is maintained, either passively, for example by “erosive recharging” (wearing that debrides old surfaces and brings out a newly-exposed, fully charged surface), or actively through manual recharging (e.g., manual debridement to expose a new surface with full activity potential, such as by abrasive polishing), or chemical recharging or reconditioning. 21 In one “self-regenerating” embodiment of the invention, recharging of surface biological activity following partial or complete “discharge” of the ionic biologically active agent initially present (e.g., after the polymer composite is newly formed and hardened) at the polymer surface is achieved by passive erosive recharging. In exemplary, “self-disinfecting” embodiments, products incorporating antimicrobially-active polymer composites of the invention may have an erodible surface and function such that abrasion of the erodible surface exposes new (originally subsurface) antimicrobial particles (active fine particles of ion-exchange resin material incorporating an ionic antimicrobial agent).

In additional embodiments of the invention, active polymer composite are provided having “rechargeable” surface structure, chemistry and biological activity after partial or complete “discharge” (including loss of structural or chemical surface active components, chemical degradation of surface active components from an original exposed surface, etc., as described above). In exemplary embodiments, the surface of a biologically active polymer composite of the invention is rehabilitated or recharged after becoming partly or completely discharged by chemical degradation, decomposition or dissociation of some or all of an initial “surface load” (e.g., surface concentration or titer of exposed metal ions, or ionic molecules, per square inch of exposed surface) or “surface activity potential” (e.g., initial biological activity, such as potential to inhibit microbial contamination, growth or effective re-transmission from the active composite surface).

Restoration the surface of a biologically stable composite material may occur following a natural wearing away by abrasion or other mechanical wearing away of the surface such as by burnishing. This may be particularly useful for antimicrobial active materials as most transfer of pathogens in hospital settings involves contact between surfaces. In this instance, the more extensive the contact, the more regenerative activity is provided. In other embodiments, restoration is provided by deliberate manual abrasion or polishing of a subject surface to remove an exhausted outer portion of the material wherein the active agent is set not only within the surface, but within the layers of the polymer surface or throughout the polymer. Abrading and polishing can be done by any number of materials such as abrasive sheets, abrasive pastes, and abrasive gels. Such abrasive and polishing materials may contain different grades of abrasive material with the finest necessary grade leaving the outer surface smooth so that there are no contaminable pores or voids.

In other embodiments, the surface of the biologically active polymer composite may be recharged chemically. For example, biologically active polymer composites comprising oligodynamic metals may become ionically exchanged in physiological fluid causing a loss of the biologically active agent. The surface of the biologically active polymer may be recharged by exposing the composite surface to an ion-rich liquid comprising a salt of the biologically active agent such as, but not limited to, silver acetate, copper chloride, or a quaternary ammonium ion disinfectant. Exposure of the surface of the biologically stable composite material to an ion-rich liquid restores about 10 to about 50% of the activity of a new surface of the biologically stable composite material, about 25 to about 75% of the activity of a new surface of the biologically stable composite material, about 15 to about 25% of the activity of a new surface of the biologically stable composite material. Such recharging may be carried out at any time however is frequently carried out when the biologically stable composite material has lost about 10%, about 20%, about 25% or more of its peak biological activity. Another effective approach to recharging of a surface, such as a composite floor, is simply to burnish the surface with a polishing wheel. This process removes microns of the upper flooring composite to reveal new particles in the composite below, thus rejuvenating the material activity.

The surface of the biologically stable composite material may additionally be activated from an original, post-fabrication unactivated state by surface chemical activation (alternatively, surface charging or chemical potentiation). In one exemplary “surface active” composite, a “Fenton reaction” is employed externally upon a finished composite surface to activate the surface (and embedded ion-exchange polymer salt components) to generate de novo hydroxyl radical from the activated surface. Within these and related embodiments, the fine particulate active polymer salt comprises and active ionic agent, such as ionically associated copper (II) or iron (II). When the polymer composite surface is sprayed, dipped or wiped with a solution of hydrogen peroxide in the presence of some ionic aqueous medium such as tap water or sodium chloride solution (0.1-0.9%), an activation chemical reaction occurs to generate hydroxyl radical at the surface of the composite, yielding a potent surface antimicrobial activation effect. Further, it is known that Fenton's reagent and hydrogen peroxide can be used to oxidize contaminants or waste waters. As such, high surface area substrates coated with oxidation-stable polymer matrixes (such as with a fluoropolymer (Teflon) or rubber such as isobutylene or styrene-isobutylene-styrene and incorporating the active Fe(II)-SCER or Cu(II)-SCER resin could be placed into holding tanks along with hydrogen peroxide to provide a means of generating hydroxyl radical in a controlled fashion while allowing the excess Fenton reagent to be easily removed from the waste water stream. In an alternative embodiment, the holding tanks can be coated with a composite incorporating the Cu(II)-SCER resin and in a cleaning step, the tank flushed with saline-H₂O₂.

In one exemplary embodiment, a port of a central venous catheter (CVC) comprising a polycarbonate (female) luer connector fitted with a silicone rubber septum and both components formulated to include fine particulate organic ion exchange powder salt in Fe(II) or Cu(II) form and at the time of pairing the female luer connector of the CVC with the male luer counterpart for the delivery of medicament or nutrition, the female portion of a luer connector is swabbed with a sponge containing hydrogen peroxide solution. The sponge may be fitted onto a male luer connector in order to allow the cap to be turned to rub/swab the hydrogen peroxide moistened sponge across the surfaces of the female luer connector thus enhancing fluid contact and the uniform generation of superoxide as a means of adequately disinfecting the inner surfaces of the connector. This embodiment is described a means of preventing catheter-related blood stream infections (CRBSIs).

In various embodiments, biologically active polymer composites of the invention can be restored, reactivated, rehabilitated or regenerated after partial or complete discharge to regain 10 to 15% of an initially-loaded, post-fabrication activity potential, 15 to 25% of initial activity potential, 25 to 50% of initial activity potential, 50 to 90% of its initial activity potential, or total initial activity potential or full “recharge” (e.g., where the same level of initial post-fabrication “loading” of functional ion-associating groups on the surface-exposed fine ion-exchange polymer salt particles are effectively “reloaded” with biologically active counter-ion, or otherwise restored (by ion-exchange or chemical reactive restoration, as described). Other means for evaluating restoration of “activity potential” include direct biological activity comparisons (e.g., Kirby-Bauer assays, adhesion assays, biofilm formation assays, colonization assays and the like), for example to test activity potential between initially loaded composites immediately after fabrication, compared with partially discharged or exhausted composites after prolonged storage, use, or exposure to environmental degradation factors (e.g., deionizing, corrosive, oxidative, hydrolytic, chemical reactive, photodegradative, or thermal degradation factors), compared with passively or self-regenerated, mechanically regenerated, or chemically regenerated, restored or rehabilitated composites.

In a related embodiment, high consistency, medical grade silicone rubber is formulated to include Ag-SCER and the silicone rubber used to formulate septa for use in central venous catheter connectors or other septum-like devices require disinfection to alleviate blood, tunnel, peritoneal, or tissue infections. In yet another embodiment, a septum is fabricated using Cu(II)-SCER and polyisoprene or other thermoplastic elastomer. In these and related devices the composite can be washed with a hydrogen peroxide-sodium chloride solution in order to generate hydroxyl radical. The presence of small amounts of free Cu(II) and hydroxyl radical is a robust means of disinfection.

Polishing of surfaces yields freshly active solutions and may be carried out at predetermined intervals. In critical environments, such as in the clinic this may be carried out on a weekly basis for example. In environments where polishing may not be possible, recharging of the surface using a simple “active” salt such as a quaternary ammonium ion disinfectant can be accomplished. In order to carry out such tasks perhaps the most logical way to approximate how much active “recharging” agent is needed is to use surface area and in conjunction with the binding capacity of the slat system to undergo recharging. For example, a 24 inch×24 inch×2 inch composite material (22 lbs.) that is formulated to include a 5 wt % additive of silver (sulfonated polystyrene-co-divinyl benzene) contains (0.05*22 lb.)*454 grams/lb.=500 grams of additive. If we assume that a micron thick slice of this composite is what requires recharging we may assume that 4% of the biologically active additive was lost and requires replacement (19.7 grams of additive). If the additive is for example a quaternary ammonium ion derivative of sulfonated polystyrene-co-divinyl benzene, we can assume that we need approximately 30% of the weight of the additive (95 mmoles) to be recharged with the ion of interest. In the case of benzalkonium chloride, this amounts to 35.3 grams or more of material that may be re-added by allowing the surface to be recharged over some period of time such as can occur when a standard disinfection procedure is carried out on the surface with a quaternary ammonium disinfectant.

In some embodiments, the self-regenerating or rechargeable composites described herein may additionally contain secondary stabilizing materials, for example antioxidants, UV stabilizers, fillers, colorants, fillers and the like.

Various assays and model systems can be readily employed to determine the effectiveness of the fractured copolymeric ion-exchange material with therapeutically useful counter-ions incorporated into polymer matrices. For example, antimicrobial effectiveness may be shown by using a Kirby-Bauer Assay. The Kirby-Bauer Assay (Disk diffusion/Zone of inhibition) is a test method that uses antimicrobial-impregnated wafers to test whether particular bacteria are susceptible to specific antimicrobial agents. In this method, bacteria are grown on agar plates in the presence of samples containing relevant antibiotic agents. If the bacteria are susceptible to a particular antibiotic, an area of clearing surrounds the sample where bacteria are not capable of growing (referred to as a zone of inhibition).

Kirby-Bauer assays can be used to provide an indication of the effectiveness of the materials (ion-exchange material loaded with oligodynamic metal ions and ammonium ions, and blended silicone LSR materials) and the materials can be shown to possess broad antimicrobial capability against Gram-negative and Gram-positive organisms, and fungi including but not limited to: Staphylococcus, Pseudomonas, Escherichia coli, Klebsiella pneumoniae, Legionella, Mycobacteria, Streptococcus, Acinetobacter, Hemophilus, and Enterococcus. As well as Aspergillus. These agents can be tailored to address multidrug resistant organisms and a variety of airborne pathogens including Mycobacterium tuberculosis, Mycobacterium abscessus, and Legionella pneumophila.

The true antibacterial or antifungal efficacy may additionally be demonstrated through the use of ISO 22196. ISO 22196, Measurement of antibacterial activity on plastics and other non-porous surfaces, has been utilized for the evaluation of antimicrobial ion-modified resins incorporated into a variety of different materials. These antimicrobial ion-exchange modified materials have demonstrated between 3-Log to 7-log overall reductions in bacterial (organism) counts for species such as Escherichia coli and Staphylococcus aureus at as little as 1.0 wt % loading levels (% by weight of active fine particulate polymer salt per final composite weight, determined prior to mixing of polymer salt with thermoplastic or thermoset polymer).

Efficacy of the biomaterials provided herein may be demonstrated, for example, by the ASTM E2180 assay (ASTM International, West Conshohocken, PA, 2007). ASTM E2180 is a method whereby treated test samples are inoculated with the test organism mixed within a semi-solid agar “slurry” to facilitate surface interaction. The test organism is thus exposed for attachment/colonization on the surface of the test material typically for 24 hours. Control samples of the same material that are not “active” according to the invention (e.g., a silicone polymer that does not contain active fine particulate polymer salt material) is similarly inoculated and tested. The test and control samples are then treated with a neutralizing solution comprising tryptic soy broth (base), lecithin (1.0 gram/liter) and Tween 80 (7.0 grams/liter). With this solution, cationic antimicrobial agents are neutralized in order to prevent them from continuing to eliminate bacteria during the test procedure, the surfaces are subsequently washed and samples are quantitatively assayed for antimicrobial activity (e.g., bactericidal and/or bacteriostatic activity). The resulting plates are incubated, and the number of survivors can be enumerated by direct surviving cell counts and/or by determining both survival and viability for reproduction through subsequent detection of colony production (colony forming units or CFUs). This provides for measurement and expression of “decontamination efficacy” of the novel biomaterials of the invention, which may be expressed as a percent reduction of viable microbes capable of surviving and/or reproducing. These values are determined for both test and control materials, and on this basis relative efficacy values for “decontamination activity”, bactericidal and/or bacteriostatic activity, and “transfer risk reduction”, among other measures of efficacy, can be determined. Comparable assays are routinely implemented to determine antifungal (fungicidal and fungistatic) activity, antiviral activity, and antiprotozoan (e.g., amebicidal) activity.

Common test organisms utilized in methods for determining antibacterial activity include Escherichia coli and Klebsiella pneumoniae. Exemplary antimicrobial polymer composites of the invention have been tested and shown to effect 3.69 and 3.72 log reductions against these bacteria, respectively. In other exemplary embodiments, antimicrobial polymer composites having as low as 1.0 wt % loading of the composite with fine particulate active ion-exchange polymer salt have been tested and shown to effect 6.2 and 5.98 log reductions in these respective organisms at as little as 1.0 wt % loading. The data from these and other assays demonstrate the ability of active ion-exchange polymer salts and polymer composites incorporating these novel materials as potent drug delivery and surface-active biomaterials for use in clinical, industrial and other applications. Efficacy may additionally be demonstrated through using a biofilm adhesion assays. In addition, the antiviral efficacy of a surface described herein may be, for example, demonstrated by the ISO 21702-19 assay (Measurement of the antiviral activity on plastics and other non-porous surfaces, International Standards Organization, Vernier Switzerland) In certain aspects of the invention, biological activity potential of active polymer composites can be varied by selecting different effective loading amounts of particle/powder distributions within composites for the active, fine particulate ion-exchange polymer salt. Biologically effective amounts (or ratios) of the polymer (e.g., per wt % of its incorporation within polymer composite mixtures) can be selected across a broadly validated range. For example, polymer composites comprising as little as 1 wt %, to as much as 75 wt % or higher, of the fine particulate ion-exchange polymer provide active composites with acceptable structural, cosmetic, stability, and performance characteristics. In certain embodiments, a selected weight percentage of the fine particulate ion-exchange polymer salt incorporated within useful polymer composite mixtures are selected as a “biologically effective amount” (by wt %) to mediate a specific biological activity potential (translatable to all biological activities described herein). In exemplary embodiments, an effective amount of a fine particulate ion-exchange polymer salt incorporated within a polymer composite may mediate antimicrobial activity potential characterized by an ability of the polymer composite to inhibit specific microbial survival, growth and/or transmission potential to a second surface or living subject. For example, effective amounts of fine particulate ion-exchange polymer salts in certain polymer composites will increase zones of bacterial inhibition by 10%, 20%, 30%, 50% or greater, up to 75-90%, or 95% or greater, compared to comparable inhibition activity measures determined for a non-active composite (i.e., a like composite not incorporating active fine particulate ion-exchange polymer salt-either having no particulate polymer material, or having like particulate ion-exchange material in like amounts not active by incorporation of biologically active ionic agent). In other embodiments, effective amounts of fine particulate active ion-exchange polymer salt will mediate inhibition of bacterial biofilms, bacterial reproduction, and/or bacterial transmission from a contaminated composite surface to a secondary surface or live subject by 10%, 20%, 30%, 50% or greater, up to 75-90%, or 95% or greater. Comparable levels of selectable activation potential for all activities imparted to the novel polymer composites of the invention (e.g., antifungal activity, antiviral activity, anti-inflammatory activity, etc.) are similarly achieved using selectable effective amounts of fine particulate polymer salt materials within different active composites, according to the description herein.

Activated polymer composites of the invention can be formed as flexible or rigid biomaterials in virtually any shape, size, thickness or structural relationship with other materials (e.g., Teflon, nylon PTFE, stainless steel, titanium, etc.) to make biomedical articles, tools and devices. The polymer composites may incorporated into biomaterials, textiles and articles of manufacture, for example, by casting, molding or assembling the composites directly into an article of manufacture, coating or laminating the composites over articles of manufacture, or mixing the composites with textiles or other precursors of articles of manufacture, among other fabrication modes and formulae.

Accordingly, the biologically active polymer composites of the invention are useful to form integral, internal or external components, infused or permeated media, lattices and textiles, laminates and coatings, etc., to provide novel structural and biological advantages to a diverse array of medical, veterinary, dental, orthopedic and laboratory materials, devices equipment and furnishings. The novel biomaterials and composites of the invention may make up the products in their entirety by molding, curing, or other fabrication means, or they may be coated, laminated, over-molded, or coextruded onto other materials, components or products or onto the same composite at a different concentration. In exemplary embodiments, components and products are made from active polymer composites of the invention by transfer molding, extraction molding, extrusion molding, blow molding, or other molding techniques. In other exemplary embodiments, biomaterials and articles of manufacture are produced by forming the solid composites as sheets, which may in turn be applied to or adhered to a different material, substrate, component or product. Coatings comprising biologically active polymer composites of the invention may have the same thickness over an entire material or product profile or surface or be coated onto a material or product in varying thicknesses at different sites or functional parts, depending on use.

The invention thus provides a valuable assemblage of biologically active polymer composites for construction of clinical, therapeutic and diagnostic materials and devices. Operative embodiments employ the biologically active polymer composites of the invention incorporated within such diverse materials and devices as antimicrobial disposable blotters, sponges, and surgical wear (e.g., gloves and shoe covers), permanent or temporary coverings for traditional fomite surfaces such as surgical trays, operation room (OR) equipment, drug and fluid delivery devices, catheters and tubing, cardiovascular and orthopedic implants, stents, grafts, and anchoring or suturing materials and devices (e.g., pins, posts, staples, and sutures) and a diverse array of comparable laboratory equipment (e.g., materials, components, tools, containers, disposable and non-disposable coverings and textiles for use in forensic, diagnostic, microbiological and tissue culture laboratories).

Additional biomaterials, components, coatings, devices, furnishings and equipment in which the novel active polymers of the invention are beneficially incorporated include, for example, food-processing equipment, food packaging/transfer materials such as liquid transfer tubing, packaging and products; consumer clothing and apparel; first responder protective wear and gear; athletic (e.g., sports therapy and gymnasium) materials, equipment and clothing; lavatory materials, furnishings and equipment, transportation equipment (e.g., high-contact/heavy use surfaces on buses, subways, trains, planes, cruise ships), and HVAC and other air and fluid circulation and management systems and components (e.g., coatings on air ducts, connectors, ports, collectors, fan blades and housings, impellers and filters made from nonwoven substrates).

Exemplary medical, laboratory and industrial materials and devices of the invention include active polymer composites integrated within paints, floor coverings and coatings, wall materials, joining and adhesive compounds for medical devices, walls and furnishings, countertops, laminate materials, filters, and appliances. These exemplary materials may be formulated as solvent-based lacquers, water-based latexes, epoxies or polyurethanes, or as two-part curing systems that can include epoxies and polyurethanes to include foams. In one detailed example, a highly durable surface coating/paint for vehicles, storage containers, aircraft, watercraft, or other comparable painted equipment are formulated to include Cu(II) or Fe(II) SCER. These coatings facilitate decontamination of surfaces, for example following a Chem/Bio warfare attack. While remaining in the field, the equipment is first treated with a salt solution of at least 20 millimolar, i.e. 0.020 moles (1.17 grams) of NaCl (58.45 grams/mole) and subsequently the surface is treated with hydrogen peroxide solution (≥3 wt %, 8 wt % of in aerosolized vapor of approximately 35 wt %). The sodium ion facilitates liberation of Cu(II) and the peroxide acts in conjunction with Cu(II) to form hydroxyl radical. The Fenton reagent has been utilized extensively for remediation of polluted soil for example, particularly using Iron (II). These coatings and materials are also useful in vehicle paints (epoxy-based) and may be employed for protective gear, air and water filters and the like.

In another exemplary embodiment, hospital rooms, cruise ship areas, prison units and other locations with surfaces/facilities at risk of colonization by infectious organisms, can be constructed or modified using antimicrobial paints or coatings of the invention (e.g., to provide antimicrobial painted walls, epoxy flooring, powder coated fixtures, etc.) incorporating Cu(II) or Fe(II) SCER or other biologically active additives of the invention. A machine that can vaporize 35% hydrogen peroxide and approximately 10-20 millimolar sodium chloride solution can broadly activate the subject surfaces to generate hydroxyl radical and eradicate even persistent organisms including Candida aureus, also known as C. aureus, a drug resistant organism that has demonstrated persistence against hydrogen peroxide vapor alone.

In yet another exemplary embodiment, ceiling tiles coated with a polymer incorporating the Cu(II)-SCER or tiles fabricated wholly from polymeric materials incorporating Cu(II)-SCER can be activated with tap water, saline or saline with dilute hydrogen peroxide in order to address Candida auris organisms that have taken up residence in the pores of the material. In another representative embodiment, a plumbing system can incorporate PVC pipe components (e.g. P-traps) fabricated from Cu(II)-SCER PVC compositions. When exposed to salt water and hydrogen peroxide, the resulting hydroxyl radical can eradicate biofilm built up on the surfaces.

Exemplary medical and laboratory devices and equipment that can be partially or completely constructed of the novel biomaterials provided here include drug and fluid delivery and catheter tubing, molded components, coatings, surgical tools and equipment, biohazard disposal surfaces and containers, hospital bedding, gurneys, stretchers, textiles including surgical scrubs, gowns, surgical drapes, bedding, wound dressings, etc. Other, similar assemblages of materials, devices and applications are contemplated for food harvesting, handling, processing and serviced industrial tools, textiles and equipment, and for heating, ventilation, and air conditioning (HVAC) system components including filters, heat exchangers, coils, duct work, fans, humidity control components, heat pumps, vents, manifolds and the like. Yet additional materials, devices and applications will incorporate the active polymer composites of the invention within bulk storage containers, public transportation surfaces, office equipment, food conveyers, clean rooms, consumer products (toys, highchairs, bathroom cleaning appliances, sexual aids, hygiene implements such as toothbrushes, dental floss and skin and eye care materials, vaporizers and other devices), automobile components (steering wheels and covers), and mobile phone covers to include protective covers or molded phone components.

Exemplary medical and hygiene products that will beneficially incorporate biologically active polymer composites of the invention include, for example, catheters, tracheostomy tubes, wound drainage devices/catheters, stent, implants, introducers, stylets, sutures, shunts, gloves (latex, neoprene, viton), condoms (polyurethane, latex, silicone), contact lenses, devices used within the eye such as to relieve pressure associated with glaucoma, gastrostomy tubes, cardiovascular stents, prostheses, pacemaker and ICD pulse generators, grafts, valves and implants, surgical guidewires, urine collection devices including drainage bags and the attached PVC tubing used in the drainage system for example to yield an entire drainage system capable of sterilizing urine, medical tubing, intravenous catheters, urinary catheters, Foley catheters, vascular access and dialysis catheters, peritoneal dialysis catheters, pacemaker leads, urological catheters, wound dressings, medical sheeting, endotracheal tubes, tracheostomy tubes, septa used for piercing with needles for sterile retrieval of drugs from supply vials, or for delivery of drugs, nutrients, saline or other materials via intravenous connectors, clamps, shunts, catheter ports, hubs, catheter port cleaning cap devices (for ensuring that septum and port are sterile for the providing drug therapy, nutrition, or removing body fluid), surgical repair constructs and meshes, and many other materials and devices.

Exemplary wound dressings of the invention include calendared silicone sheet composites incorporating active, fine particulate ion-exchange polymer salts, such as a silver ion exchange salt. The composite calendared sheet may be attached to a release liner, and the sheet may be fenestrated to allow transmission of water from a wound bed when the dressing is in contact with wound tissue (FIG. 14). These and related dressings are particularly effective for dressing burns, as the silicone is non-adherent, while the presence of the active, fine particulate ion-exchange polymer salt precludes the attachment and survival of bacteria and fungi. Another example includes a polyurethane or thermoplastic elastomer foam incorporating a Cu(II) weak cation or Cu(II) strong cation exchange resin.

Exemplary polymer film-based devices of the invention include PVC urine drainage bags comprised of PVC composite incorporating approximately 0.75 wt % Ag(I)-SCER. Drainage bags are known to be a source of infection for patients where the reflux of urine back into the bladder leads to infection. The Ag(I)-SCER in PVC has been shown to be highly effective against a broad array of pathogens at loadings of 0.5 wt % and higher.

Exemplary sexual aids include dildos, vibrators, sleeves and other stimulatory devices, male and female sex toy products, rings, as well as any penetrating or penetrable adult sexual device fabricated from silicone, polyurethane or other soft flexible hypoallergenic material. Exemplary contraceptive devices that will benefit from the inclusion of biologically active polymer composites of the invention include intrauterine devices (IUDs) comprising a Cu(II) derivative form of a strong or weak cation exchange resin. Paragard® is a commercially available IUD that releases small amounts of Cu⁺⁺ from a copper filament and is known to be safe.

Another exemplary contraceptive device embodiment includes sponges that releases benzalkonium and cholalic acid (cholate) for placement into the vaginal tract. The high surface area device is conducive to having active fine particulate polymer salt additives incorporated without having any effect on the mechanical performance of the device.

In yet another exemplary embodiment of a vaginal sponge, the acid form of sulfonated polystyrene divinylbenzene or the acid form of the polymethacrylic acid-co-divinylbenzene active fine particulate polymer salts may be added to a flexible polymer matrix as a means of having an effect on the local pH within the vaginal tract. This will allow for the high surface area sponge to generate hydronium (Note: benzene sulfonic acid has a dissociation constant of 10³) which will affect the local pH (decreasing) at the entrance to the cervix. Because sperm require high pH in order to function properly, such a device will decrease sperm motility. Silicone and or thermoplastic elastomers are appropriate materials for such an application. The same strategy can be applied to a diaphragm noting that silicones and polyurethanes are the appropriate materials for diaphragms and sponges.

In yet another exemplary embodiment, active fine particulate polymer salts modified to include spermicidal agents such as benzalkonium and/or cholalic acid and the additives blended into a flexible polymer matrix such as latex and condoms can be fabricated by a dipping process. In some embodiments only the outer layer or layers may include the spermicidal agent depending upon the number of dipping processes required to produce the condom.

All exemplary contraceptive devices described in this invention can be further modified to include antiviral agents to minimize the likelihood of transmission of HIV during intercourse.

Among significant industrial and public utilities uses, the biologically active polymer composites of the invention are particularly well adapted for useful integration in air and water-handling systems, including heating, vacuum, and air conditioning (HVAC) components, conduits, fittings, filters, recirculators, pumps and the like. The heating, vacuum, or air conditioning components can include one or more of duct work, heat exchange coils, heat exchangers, fan components, vents, energy-recovery ventilators, blower components, ballasts, levers, air filters, water filters, heat pumps, fluid handling systems and/or the like. Coatings incorporating organic ion exchange polymer salt materials embedded within the composite (or composite surface layer(s)) require activation by a salt with which exchange can occur. As such, in environments where the moisture present is condensate of atmospheric humidity, ion concentration may be exceptionally low or non-existent. Thus, for situations where this is possible, the coatings can incorporate a sacrificial salt such as an alkali salt of a dioic acid, sodium carbonate, a poly-sodium phosphate such as disodium phosphate or trisodium phosphate, or sodium formate depending upon the needed temperature operating range for processing (i.e. where the sacrificial additive will not melt) an aluminum phosphonate, or a non-corrosive salt such as sodium gluconate so as to provide sodium or other ion for exchange with a cation of a biologically active fine particulate polymer salt. The aforementioned examples are not an exhaustive list of sacrificial salts that can provide this benefit.

In other embodiments, the biologically active polymer composites of the invention are uniquely adapted for improving safety and performance of building, flooring and surface construction materials, including hospital, laboratory and home building, construction and sealing and adhesive materials. Among such materials that will beneficially incorporate surface paints or coatings of these active polymer composites are flooring materials, countertop materials, wall construction materials, caulking compounds, foaming insulation materials, and cast or molded enclosures for bathroom applications. One exemplary use for these embodiments will be to fight toxic mold (Stachybotrys chartarum) and other species of fungi such as Alternaria, Aspergillus, Candida auris, Aureobasidium, Chaetomium, Cladosporium, Fusarium, Mucor, Trichoderma, Ulocladium, and Penicillin that can take up residence in homes, hospitals, extended care facilities, temporary (disaster) housing, or barracks housing personnel in environments conducive to the proliferation of toxic bacteria, fungi, viruses or other microorganisms e.g., by coating indicated building materials or structure surfaces with paints or durable coating compositions integrating active ionic agents such as Cu(II)-SCER.

In another exemplary embodiment, a medical device adhesive comprising a silicone, urethane, or vinyl compound is formulated to include Ag-SCER, Zn(II)-SCER, or a quaternary ammonium-SCER in order to protect the boundary between joined materials.

With regard to construction of biologically active textiles, the polymer composites of the invention can be used to construct finished fabrics derived from naturally occurring fibers or man-made materials, or from plant-based materials such as paper. The fabric materials can be constructed from one or more of a weave, knit, knot, crochet, or melt spun or unwoven (nonwoven fabrics) and the antimicrobial additives of the present invention can be incorporated by inclusion into the fibers of manmade material prior to fabrication of yarn, thread or the like or the antimicrobial additives of the present invention may be added into a polymer prior to spinning or melt blowing or with coating (sizing) a fabric. The textiles as described herein may be utilized to fabricate any variety of textile-based products to include clothing and garments such as shirts, socks and stockings, and pants that may find applications for example in sportswear, and military applications. Garments for use in hospital and healthcare environments may include surgical scrubs, neckties, and lab coats, as well as hospital gowns, pajamas, undergarments or diapers for example. Other textile-based articles can include surgical masks, booties, and protective suiting for application in and around infectious diseases.

In other embodiments, the self-disinfecting compositions may be used to make touch surfaces for use in one of a clinic, hospital, nursing home, long-term care facility, gymnasium, sporting facility, workout facility, kitchen, bathroom, recreation center, academic institution, cafeteria, watercraft, motorized vehicle, and/or disposal container. Touch surfaces as related to gymnasiums, recreation centers, and sporting institutions can include for example grips related to equipment and exercise machines, mats for stretching, martial arts, boxing, and wrestling.

In other exemplary embodiments the active fine particulate polymer salts may be incorporated into adhesives and sealers for use in building construction materials in order to impart surface or bulk antimicrobial properties to the materials. For example, roofing materials may be susceptible to fungal growth and/or rot. Thus, the incorporation of a fungicidal active fine particulate polymer salts, such as Cu(II)-SCER can alleviate such problems.

Further embodiments of the invention include novel marine paints that are effective to prevent or eliminate attachment of marine biofilms and macrofouling life forms such as crustaceans and mollusks, which increase drag and decrease marine vessel performance, raising fuel costs and increasing the price of marine-shipped goods. Marine biofouling includes unwanted deposition, attachment and accumulation of microorganisms, plants and animals on ship hulls and other surfaces exposed to sea water or spray. Fouling of ship's hulls and other surfaces imposes extraordinary performance losses and excess fuel costs, reduces shipping speed and efficiency, and requires frequent, expensive dry-docking, cleaning, re-coating and in some cases costly repairs.

Processes of biological fouling typically involves discrete stages, including the accumulation of adsorbed organics, settlement and growth of bacteria creating a biofilm matrix, and subsequent succession of micro- and macrofoulers. Microfouling involves the formation of biofilm and adhesion to the surface, and macrofouling refers to the attachment of organisms such as barnacles, diatoms, mollusks and seaweeds that produce a fouling community. The growing bacteria and the chemicals they secrete make up microfouling, also referred to as ‘slime’, which develops within hours of an object's immersion in water. Within a few days, macrofouling develops as unicellular eukaryotes, such as protozoa and diatoms, that colonize the surface. Multicellular eukaryotes begin colonizing the surface within several weeks, beginning with settlement of planktonic larvae and algal spores.

Marine antifouling paints, also known as bottom paints, are a specialized category of coating formulations made with cuprous oxide (or other copper compounds) and/or other biocides—special chemicals which impede growth of barnacles, algae, and marine organisms. There are three main types of antifouling technology: controlled depletion polymer (CDP), self-polishing copolymer (SPC) and foul release (FR). The rosin-based CDP technology is now the most basic antifouling coating in the industry. Water migrates into the CDP paint film which in turn dissolves rosin and biocides, which leach into the sea. As undissolved biocide and rosin remains in the leached layer, the dissolution rate continuously declines, and the remaining material (coating) becomes less predictable and less effective. By substituting for the rosin/biocide combination using Cu(II)-WCER, a coating that is reliant on ion exchange to liberate the active Cu(II) There are a variety of coating types that these additives can be used in include epoxies, polyacrylates, polysiloxanes and the like. The zinc (II) form of the resin can also be incorporated as a slow-release biocide.

In other exemplary embodiments of anti-fouling coatings provided herein, active fine particulate polymer salts of the invention can be incorporated into surface materials such as Dupont's Corian, an acrylic polymer containing alumina trihydrate. The use of a strong or weak cation exchange resins with Cu(II) modification are contemplated to provide surfaces that are at least as antimicrobial as copper metal surfaces. In order to facilitate disinfection of such a surface, a light misting of the surface with a saline solution will liberate enough Cu(II) to facilitate the eradication of bacterial and fungal organisms and the subsequent addition of hydrogen peroxide can further aid disinfection by the production of hydroxyl radical. In another embodiment, epoxy can be substituted for acrylic to provide a tough composite material that can have similar applications.

In other exemplary embodiments the active fine particulate polymer salts may be incorporated into paints and other durable coatings that can be applied inside clinical institutions, long-term care facilities, particularly in rooms that house patients with drug-resistant infections (e.g. burn units). In particular, the addition of Cu(II)-SCER is demonstrated to be remarkably effective when formulated into acrylic latex enamel paint at a concentration of roughly 1.5 wt % (approximately 0.18 wt % Cu(II)), for a resin of 12 wt % Cu(II). The resulting coatings are easy to tint at this low concentration. Similarly, the coatings can be applied on cruise ships where norovirus outbreaks are common and in hospitals, restaurants, ambulatory centers, long-term care facilities, war ships, submarines, aircraft and other assets where humans and animals may be susceptible to exposure of novel viruses such as SARS-COV-2. Cu(II) has been shown to be active against norovirus (non-enveloped). In the event of an outbreak, the use of tap water, saline, or standard ionic disinfectant such as a quaternary ammonium salt solution over the coated area will liberate small amounts of Cu(II). The addition of hydrogen peroxide (H₂O₂) to the composite surface liberates hydroxyl radical (HO·) in the presence of Cu(II) to rapidly disinfect inactivate microbes, biofilm, and viruses.

In other exemplary embodiments, silicone, polyurethane or thermoplastic elastomers such as C-Flex (styrene-ethylene-butylene-styrene) (SEBS) or styrene-isobutylene-styrene polymer (SIBS) are combined with micronized strong cation exchange resin (USP) in sodium form. The polymer sulfonated styrene-ethylene-butylene-styrene sulfonated SEBS deters formation of thrombus (e.g., when used as a construction material or coating for stents) as a result of the prevalence of sulfonate groups. Other examples of the strong cation exchange resin (SCER) can include reduced glutathione (γ-L-glutamyl-L-cysteine-modified resin. This cysteine derivative is known to have anti-inflammatory effects. Similarly, the SCER acid form can be combined with azathioprine, an antiproliferative agent, and the resulting complex reduced to small particle size and the material (powder) incorporated into the stent coating.

In other examples, the combination of antimicrobial ion exchange resins into materials can be used in securing prosthetic devices, such as prosthetics socks, stump shrinkers, and prosthetic liners for amputees, can minimize the presence of Staphylococcus (epidermidis and aureus) and Propionibacteria, microbes of normal human flora. In addition, Bacillus subtilis and fungal organisms known to cause athlete's foot that include Epidermophyton, Trichophyton, and Microsporum.

In yet another example, the combination of antimicrobial ion exchange resin, such as the silver form of a strong cation exchange resin, into silicone can be calendered into a sheet, fenestrated to allow the transmission of wound fluid, cut to size, packaged and sterilized by ETO or other appropriate sterilization method. The resulting wound dressing acts as a contact layer for burn or other wound management and the presence of Ag-SCER prevents biofilm from accumulating.

Similarly, socks and sheaths are also provided with gel attached to, or sandwiched between, the fabrics. The gel is usually made of silicone and provides excellent cushioning, pressure distribution and reduced friction. The thickness and stiffness of the gel will dictate the cushioning qualities of the sock. Because gel tends to flow from areas of high pressure to areas of lower pressure, maintenance of a more even pressure distribution is possible. If the sock or sheath is constructed with the gel exposed, the gel should be worn against the skin. This will help protect the skin from the friction forces created during walking, since the motion will tend to occur between the gel sheath and the prosthesis rather than between the gel and the skin. If the gel is formulated with a small percentage of Ag-SCER (0.5-1.0 wt %) bacterial growth is deterred.

Synthetic fibers that can be spun at or below 250° C. are exemplary materials for inclusion of modified SCERs of the invention. Cu(II), Ag, and Benzalkonium (BA), cetylpyridinium (CP) or other quaternary ammonium modified resins can be incorporated into various fibers whether for weaving fabrics or the production of nonwoven materials.

In certain embodiments, silicone composite comprising Ag-SCER (0.5-3.0 wt %) is fabricated into a polyurethane (or other appropriate polymer such as a TPE) sizing solution that can be applied to fabrics to protect against bacterial and fungal growth, formulated directly into the gel portion of the prosthetic sock or can be fabricated directly into a liner for use with the prosthetic.

Polyurethane, TPE or other polymer foams/fabrics can additionally be modified with Ag-SCER, Cu-SCER, or BA-SCER or combinations thereof to alleviate foot odor and bacterial organisms that lead to foot odor. In exemplary embodiments, Cu-SCER is a primary agent incorporated into the shoe insert because of its efficacy against fungi. In related embodiments, mattresses comprising polyurethane or other polymer foams incorporating antimicrobial SCERs protect these and other furnishings against contamination due to soiling in hospitals and nursing homes. In addition to the core of these furnishings, such as a foam core, the fabric used for covering beds, chairs and sofas etc. can also integrate antimicrobial materials and coatings of the invention.

In certain embodiments of the invention, antimicrobial ion exchange resin powders can be incorporated into powder coatings for the purposes of application to a variety of metallic surfaces, such as appliances, aluminum extrusions, medium density fiberboard, for example by use of thermoset or thermoplastic powder coating. The thermosetting variety incorporates a cross-linker into the formulation. When the powder is baked, it reacts with other chemical groups in the powder to polymerize, improving the performance properties. The thermoplastic variety does not undergo any additional actions during the baking process as it flows to form the final coating. The most common polymers used in powder coatings include polyesters, polyurethanes, polyester-epoxy (known as hybrid), straight epoxy (fusion bonded epoxy) and acrylics. Generally, the production of powders used for coating in this fashion are accomplished by mixing the polymer granules with hardener, pigments and other powder ingredients in an industrial mixer, such as a turbomixer, the mixture is heated in an extruder, the extruded mixture is rolled flat, cooled and broken into small chips, and the chips are milled and sieved to make a fine powder. This process is conducive with the formation of composites using fine particulate biologically active ion-exchange polymer salt materials.

In more specific embodiments of powder coatings, fusion bonded epoxy materials incorporating fine particulate biologically active ion-exchange polymer salt materials are used to coat the inner surfaces of oil and gas pipelines to prevent microbial induced corrosion. Corrosion is a significant issue in oil and gas transportation pipelines. In the United States, approximately 185,000 miles of liquid petroleum pipelines, 320,000 miles of gas transmission pipelines, and over 2 million miles of gas distribution pipelines are at risk for corrosion. Pipelines are constructed largely of corrosion-prone carbon steel, resulting in high maintenance and repair costs. Protection of iron against almost all types of corrosion can largely be achieved by applying coatings to surface. However, coatings can have a limited service life. If left untreated, corrosion can lead to material failure and the subsequent release of pipeline products into the environment. To prevent such occurrences, repairs must be scheduled. This can interrupt business and lead to long production delays. Ideally, gas pipeline infrastructure should last 50+ years while in service. NACE data suggests the industry spends ~$7B annually on direct corrosion control and repair on oil and gas pipelines. Pipelines are coated to protect metals against corrosion; however, most coatings need an improvement in an effective barrier against various corrodents and surfaces with enhanced protection against microbial induced corrosion (MIC).

A significant portion of “internal” pipeline corrosion results from MIC. Many organisms can contribute to corrosion as the primary cause with the main types being anaerobic sulfate-reducing bacteria (SRB), sulfur-oxidizing bacteria, iron-oxidizing and reducing bacteria, manganese-oxidizing bacteria, and acid-producing bacteria (APB). SRB gain energy for growth by reduction of sulfate to hydrogen sulfide and APB produce organic acids by fermentation. Bacteria can also promote external corrosion by depolarization via hydrogen consumption formed at the pipe surface by cathodic protection. While it is difficult to determine exact percentages, MIC is believed to be one of the leading causes of corrosion in the industry. Aerobic bacteria require oxygen for survival. They are present in aerated moist soil containing organic carbon sources. There are two main types of aerobic bacteria 1. The obligate aerobes that compulsorily require oxygen for deriving energy, growth, reproduction, and cellular respiration. Anaerobic bacteria are bacteria that do not live or grow when oxygen is present. In humans, these bacteria are most commonly found in the gastrointestinal tract. They play a role in conditions such as appendicitis, diverticulitis, and perforation of the bowel.

According to the National Association of Corrosion Engineers (NACE), the oil and gas industries spend ~$7B annually on direct corrosion control and repair on liquid oil and gas transmission pipelines. The industries spend ~$1.4B each year to protect or restore production and exploration infrastructure and equipment. Indirect costs associated with corrosion are service interruption, environmental cleanup and legal costs exacerbate the financial impact of corrosion.

An alternative application for a composite is its use as a thin film, solid state lithium ion battery separator (FIG. 13). In this application Li-SCER is prepared by reacting lithium acetate with the acid form of a strong cation exchange resin (sulfonated polystyrene-divinylbenzene copolymer). The lithium salt is produced in quantitative yield according to the scheme H-SCER+LiOAC_Li-SCER+HOAc. The resin is washed, filtered, and dried under vacuum. The resin is then milled to particle size of ≤10.0 micron using a centrifugal ball mill.

The building blocks of a battery are the cathode and anode, and these two electrodes are isolated by a separator. The separator is generally moistened with electrolyte and forms a catalyst that promotes the movement of ions from cathode to anode on charge and in reverse on discharge. Ions are atoms that have lost or gained electrons and have become electrically charged. Although ions pass freely between the electrodes, the separator is an isolator with no electrical conductivity. Battery separators provide a barrier between the anode (negative) and the cathode (positive) while enabling the exchange of lithium ions from one side to the other. Commercially available Li-ion cells use polyolefin as a separator. This material has excellent mechanical properties, good chemical stability and is low-cost.

The Li-ion separators of today, must be permeable and the pore size ranges from 30 to 100 nm. (nanometer), or 30-100 millionths of one millimeter or about 10 atoms thick.) The recommended porosity is 30-50 percent. This holds enough liquid electrolyte and enables the pores to close should the cell overheat.

The current invention negates the need to have a porous Li-ion separator because the Li-SCER is a solid-state electrolyte. High level (high weight percentage) loadings of Li-SCER can be achieved using various materials of the invention in order to fabricate composite lithium ion battery separators, typically using polymers that will operate above their glass transition temperatures. The glass transition temperatures should be well-below room temperature but not exclusively restricted to these materials. Useful materials within these aspects of the invention include, for example, polysiloxanes, low density and high-density polyethylene, atactic, syndiotactic, or isotactic polypropylene, poly(butyl acrylate), polyoxymethylene, a vinyl polymer, an arylene vinyl polymer, polyisobutylene, styrene-ethylene-butylene-styrene copolymer, styrene and polybutadiene. Separator thickness was targeted at 1.0-5.0 micrometer thickness. Milling of the resin allowed for the fabrication of material with a particle distribution of average 0.33 micron. At a thickness of 1.0 micron a stack of up to 3-particles could be stacked on top of one another. At Li-SCER loadings of 40-60% by weight the films are stable and flexible. Lit ion conduction measurements were made using electrochemical impedance spectroscopy (EIS).

Although dispersion of high concentrations of the Li-SCER in a polymer matrix provides indicated (electronic activity) advantages, others embodiment may include pressed solid discs (or other shapes) of the Li-SCER designed into an array. If the solid can be made into thin film without a matrix polymer (e.g., compressed into a thin film), then the electronically active Li-SCER will be in essence in essence its own matrix (i.e., the ion exchange resin crosslinked polystyrene-divinyl benzene matrix carries the lithium). Other embodiments could include porous and non-porous composites of the Li-SCER in polypropylene for example. In some examples the Li-SCER is dispersed within a gel to facilitate ion transfer between anode and cathode from the matrix.

In yet another embodiment employing Li-SCER, a controlled release delivery system for oral or implantable application is constructed from Li-SCER and a biocompatible material. Materials that can be used include silicone, polyurethane, polyacrylates, and other thermoplastic elastomers that can include polyisobutylene and styrene-ethylene-butylene-styrene other thermoplastic or castable biocompatible materials. Lithium belongs to the class of medications known as mood stabilizers. It has a narrow therapeutic index and the blood concentration of lithium is required to remain in a tight range of 2.8-8.3 mg/L. Higher levels can pose serious and even lethal toxicity to the patients, while lower levels do not provide adequate efficacy. It is taken to decrease the intensity of manic episodes or to prevent manic episodes among people with bipolar disorder. Achieving near zero-order delivery can be achieved using a porous biomaterial comprising Li-SCER and at least one biomaterial and in an alternative embodiment, a Li-WCER (weak cation exchange resin) can be incorporated into the matrix materials.

Additional supportive description pertaining to certain aspects and embodiments of the invention may be found, for example, in “Compositions And Methods For Promoting The Healing Of Tissue Of Multicellular Organisms” U.S. patent application Ser. No. 12/162,990, filed Jul. 31, 2008, PCT Patent Application Serial No. PCT/US07/02780, Jan. 31, 2007, to David Vachon, which claims priority benefit of U.S. Provisional Patent Application Ser. No. 60/764,033, filed Jan. 31, 2006; “Compositions And Methods For Promoting The Healing Of Tissue Of Multicellular Organisms” U.S. patent application Ser. No. 12/690,081, filed Jan. 19, 2010, which is a Continuation-In-Part of U.S. patent application Ser. No. 12/162,990, filed Jul. 31, 2008, which is a 371 of PCT/US07/02780, filed Jan. 31, 2007, to David Vachon which claims priority benefit of U.S. Provisional Patent Application Ser. No. 60/764,033, filed Jan. 31, 2006; and “Biologically Efficacious Compositions, Articles of Manufacture and Processes For Producing And/or Using Same” U.S. patent application Ser. No. 13/532,562, filed Jun. 25, 2012, to David Vachon, which claims priority benefit of U.S. Provisional Patent Application Ser. No. 61/501,086, filed Jun. 24, 2011, U.S. Provisional Patent Application Ser. No. 61/616,332, filed Mar. 27, 2012, each of which is incorporated herein by reference in its entirety for all purposes.

### EXAMPLES

Exemplary compositions, methods, materials and devices of the invention are provided here, which are not to be construed to limit the scope of the invention. The claims of the application are supported by the entirety of the disclosure as well as these examples.

All ion-exchange materials for use within the invention can be purified prior to, or following association with, biologically efficacious counter-ion materials described. In certain exemplary embodiments, ion-exchange materials are received from a commercial supplier and employed as received, or pre-conditioned for example by extraction with isopropyl alcohol prior to air and/or vacuum drying. All matrices such as polymer matrices used in the fabrication of the compositions such as silicone rubber, were prepared according to supplier specifications.

### Example 1

### Formation of IRP69-Ag

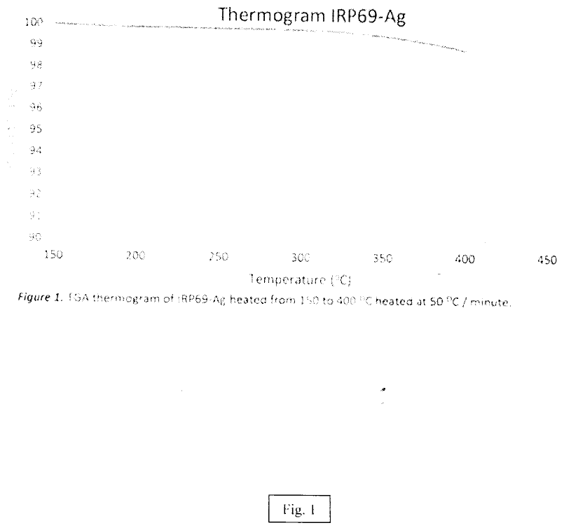

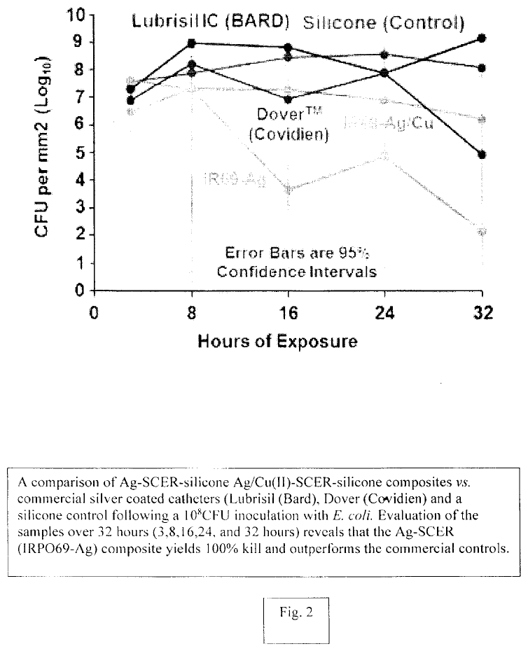

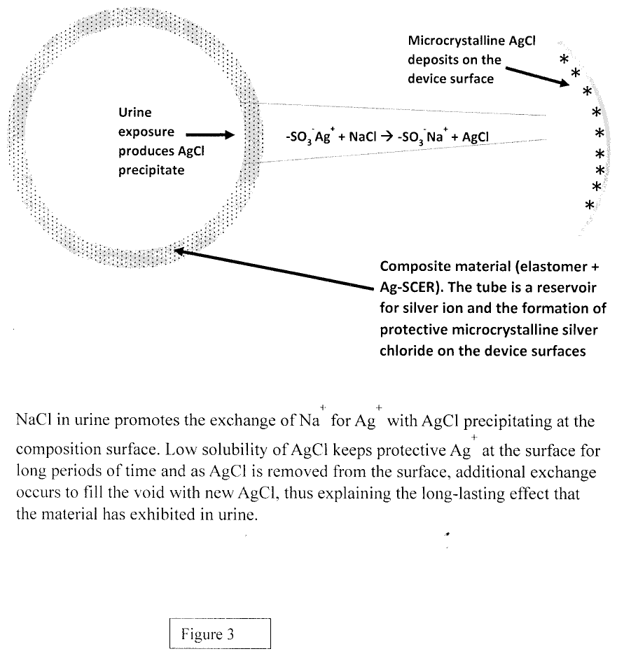

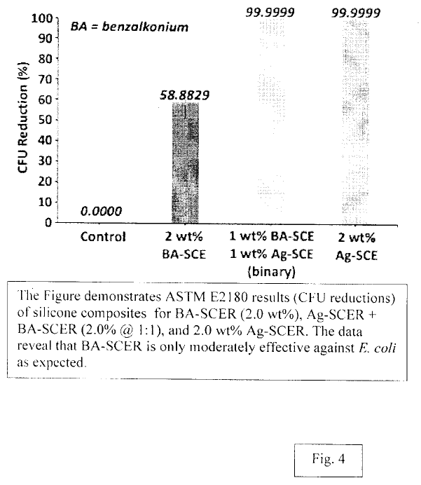

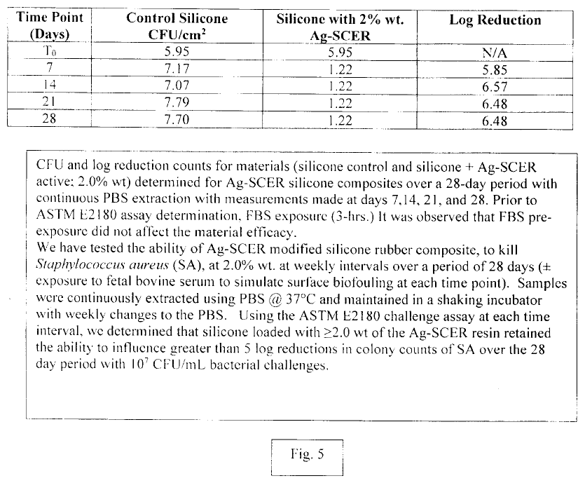

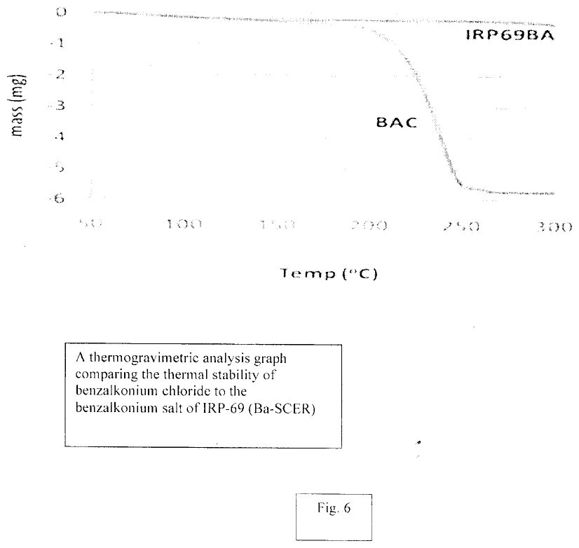

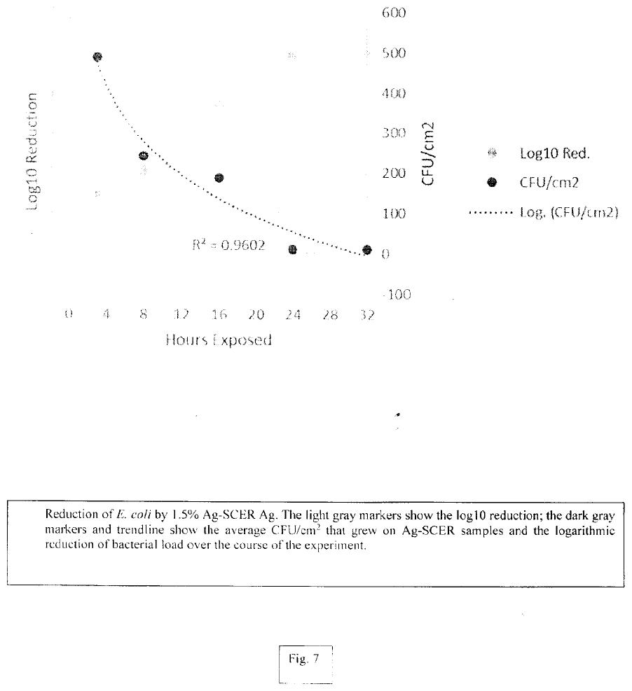

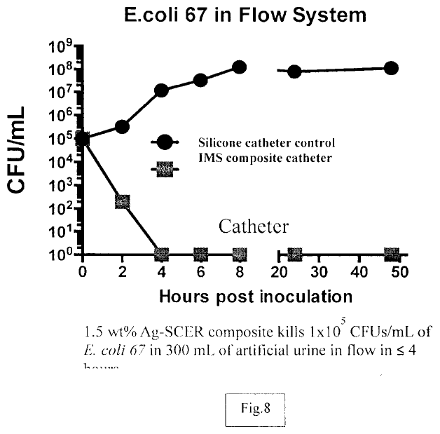

Amberlite IRP69 strong cation-exchange material was stirred in a minimal amount of deionized water and a large excess (~10-500 molar excess) of the salt (containing the cation of interest, such as benzalkonium chloride) was added and the mixture stirred by the addition of a mechanical stirrer for 60 minutes. The solid was filtered washed with copious amounts of deionized water (until the filtrate does not contain any benzalkonium chloride (for example) as evidence from ultraviolet spectroscopic evaluation of the filtrate. The active IRP69 was dried under vacuum at 130° C. and the material was milled with an IKA homogenizer and the resulting particles were put through a sieve with a 35 μm cutoff. The powder was dried under vacuum and used for incorporation within various polymer composite mixtures. Thermogravimetric analysis of IRP-69-Ag as shown in FIG. 1 demonstrates that the little degradation of IR69F-Ag below 400° C.

Another method of synthesizing the active fine particulate polymer salts involves the use of Amberlite IRP69F-H (acid form) strong cation-exchange material that can be stirred in a minimal amount of deionized water and a molar equivalent or excess amount of the acetate salt (containing the cation of interest, such as silver acetate, iron acetate, copper acetate, or organic acetates to include chlorhexidine diacetate for example). Following the addition of the acetate salt the mixture can be stirred using a mechanical stirrer for 2-24 hours depending on size of the reaction. The solid can then be filtered, washed with copious amounts of deionized water (until the filtrate does not contain the cation of interest silver acetate (for example) as evidenced from silver test strips (Macherey-Nagel, Bethlehem, PA). The pH of the wash was also monitored using pH test strips in order to gauge the presence of byproduct HOAc and the washes can be continued until the pH is neutral. The active IRP69 resin can then be dried under vacuum at 130° C. and the material was milled using a Retsch PM100CM planetary mill. The powder was dried under vacuum and used for incorporation within various polymer composite mixtures. Yields of the modified resin approach 100% (of cation exchange capacity) using this method.

### Example 2

### Preparation of IRP64-Benzalkonium

In another procedure, Amberlite IRP64-Na+ (sodium form) strong cation or Amberlite IRP64 ion-exchange material was stirred in a minimal amount of deionized water with an equal amount of isopropanol. A large excess (~10-500 molar excess) of the salt (containing the cation of interest, such as benzalkonium chloride) was added and the mixture was heated to 65° C. (optimal temperatures are between 55-70° C.) and continuously stirred with a mechanical stirrer for up to 72 hours. The active ion-exchange material was subsequently filtered and washed with isopropanol and deionized water until the filtrate did not contain any benzalkonium chloride (for example) as evinced by ultraviolet spectroscopic evaluation of the filtrate. The modified IRP64 was dried under vacuum at 130° C. and the active resin was milled using a Retsch PM100 planetary ball mill with appropriate grinding media (yielding a particulate active resin product milled to approximately 100-1000 nm particle sizes as measured by light scattering).

### Example 3

### Preparation of IRP69-Cu

Amberlite IRP69-H (acid form) strong cation-exchange material was prepared by stirring the sodium form of IRP69, IRP69-Na in an excess of 0.1N HCl. The material was filtered and washed with deionized water until the pH is neutral. The resin was titrated to determine the exchange capacity using established methods and the material is combined with an equimolar amount of copper acetate. After a period of a couple of hours the material was filtered and washed until the eluent was free of Cu as indicated by a copper test strip (BRAND). The resin was dried and milled to a particle size of 1-10-micron distribution. Elemental analysis of the compound for copper revealed copper content of 11-15 wt % by ICP-OES.

### Example 4

### Formation of Silicone Gel Containing IRP-69-Ag

MED 6345 Silicone Gel (Nusil Silicone Technology, Carpinteria CA 93013) with IRP69-Ag 12 g part A (catalyst component)+12 g part B (crosslinking component)+2.67 g IRP69-Ag (10% w/w) was mixed in Speedmixer, poured into mold and air bubbles allowed to escape (~20 min) and cured at 70° C. for 1 hr. Cure was not inhibited.

### Example 5

### Formation of Silicone Gel Containing IRP-69-Benzalkonium

MED 6345 Silicone Gel (Nusil Silicone Technology, Carpinteria CA 93013) with IRP69-Benzalkonium 3.07 g part A+3.07 g part B+0.3231 g IRP69-BA (5% w/w) was mixed by hand, poured onto release liner and air bubbles allowed to escape (~20 min) and cured at 84° C. for 23 min. Cure was complete and not inhibited by the ammonium compound.

### Example 6

### Formation of Silicone Gel Containing IRP-69-Cetylpyridinium

MED 6345 Silicone Gel (Nusil Silicone Technology, Carpinteria CA 93013) with IRP69-Cetylpyridinium 3.02 g part A+3.02 g part B+0.3179 g IRP69-BA (5% w/w) was mixed by hand, poured onto release liner and air bubbles allowed to escape (~20 min) and cured at 84° C. for 23 min. Cure was complete and not inhibited by the ammonium compound.

### Example 7

### Formation of Silicone Gel Containing IRP69-Octenidine

MED 6345 Silicone Gel (Nusil Silicone Technology, Carpinteria CA 93013) with IRP69-Octenidine 3.015 g part A+3.015 g part B+0.3174 g IRP69-BA (5% w/w) was mixed by hand, poured onto release liner and air bubbles allowed to escape (~20 min) and cured at 84° C. for 50 min. Cure was complete and not inhibited by the ammonium compound.

### Example 8

### Formation of Liquid Silicone Rubber with IRP69

MED-4955 liquid silicone rubber (Nusil Silicone Technology, Carpinteria CA 93013) with IRP69-Ag 8 g part A+8 g part B+0.4948 g IRP69-Ag (3% w/w) was mixed in Speedmixer, spread on release liner and placed in vacuum oven at room temperature to remove air bubbles. Another release liner was placed on top, rolled with a jar to flatten and then cured (~80° C., ~10 min). Cure was complete.

### Example 9

### Formation of Liquid Silicone Rubber with IRP69-BA

MED-4955 liquid silicone rubber (Nusil Silicone Technology, Carpinteria CA 93013) with IRP69-BA 18 g part A+18 g part B was mixed in Speedmixer, 6.0844 g removed and 0.3202 g IRP69-BA (5% w/w) mixed in by hand, spread on release liner and placed in vacuum oven at room temperature to remove air bubbles. Another release liner was placed on top, rolled with a jar to flatten and then cured (~80° C., ~10 min). Cure was complete and not inhibited by the ammonium compound.

### Example 10

### Formation of Liquid Silicone Rubber with IRP69-CP

MED-4955 liquid silicone rubber (Nusil Silicone Technology, Carpinteria CA 93013) with IRP69-CP 9 g part A+9 g part B was mixed in Speedmixer, 6.095 g removed and 0.3208 g IRP69-CP (5% w/w) mixed in by hand, spread on release liner and placed in vacuum oven at room temperature to remove air bubbles. Another release liner was placed on top, rolled with a jar to flatten and then cured (~80° C., ~10 min). Cure was not inhibited.

### Example 11

### MED-4955 Liquid Silicone Rubber with IRP69-Octenidine

MED-4955 liquid silicone rubber (Nusil Silicone Technology, Carpinteria CA 93013) with IRP69-Octenidine 12 g part A+12 g part B was mixed in Speedmixer, 6.041 g removed and 0.318 g IRP69-Oct (5% w/w) mixed in by hand, spread on release liner and placed in vacuum oven at room temperature to remove air bubbles. Another release liner was placed on top, rolled with a jar to flatten and then cured (~80° C., ~10 min). Cure was not inhibited.

### Example 12

### Formation of UV-Curing Liquid Silicone Rubber with IRP69-Ag

Momentive 2060B UV-Curing liquid silicone rubber (Momentive Performance Materials, Albany, NY 12211-2374) with IRP69-Ag 41.9 g part B (crosslinking component)+1.52 g UV catalyst (photoinitiator) was mixed in Speedmixer, 7.16 g removed and 0.3768 g IRP69-Ag (5% w/w) mixed in by hand, spread on release liner and placed in vacuum oven at room temperature to remove air bubbles. The material was passed through a UV curing system (Fusion UV Systems, Inc.) at 4 ft/min with each side of the gel exposed to UV lamp once. Cure was not inhibited.

### Example 13

### Formation of UV-Curing Liquid Silicone Rubber with IRP69-BA

Momentive 2060B UV-curing liquid silicone rubber (Momentive Performance Materials, Albany, NY 12211-2374) with IRP69-BA 41.9 g part B+1.52 g catalyst was mixed in Speedmixer, 7.28 g removed and 0.383 g IRP69-BA (5% w/w) mixed in by hand, spread on release liner and placed in vacuum oven at room temperature to remove air bubbles. The material was run through a UV curing system (Fusion UV Systems, Inc.) at 4 ft/min with each side of the gel exposed to UV lamp once. Cure was not inhibited.

### Example 14

### Formation of UV-Curing Liquid Silicone Rubber with IRP69-CP

Momentive 2060B UV-Curing Liquid Silicone Rubber (Momentive Performance Materials, Albany, NY 12211-2374) with IRP69-CP 41.9 g part B+1.52 g catalyst was mixed in Speedmixer, 7.11 g removed and 0.374 g IRP69-Ag (5% w/w) mixed in by hand, spread on release liner and placed in vacuum oven at room temperature to remove air bubbles. The material was run through a UV curing system (Fusion UV Systems, Inc.) at 4 ft/min, requiring 4 passes before gel could be removed and turned over, then one more pass on the reverse side. Cure was not inhibited.

### Example 15

### Formation UV-Curing Liquid Silicone Rubber with IRP69-Oct

Momentive 2060B UV-curing liquid silicone rubber (Momentive Performance Materials, Albany, NY 12211-2374) with IRP69-Oct 15 g part B+0.544 g catalyst was mixed in Speedmixer, 6.3327 g, removed and 0.3333 g IRP69-Oct (5% w/w) mixed in by hand, spread on release liner and placed in vacuum oven at room temperature to remove air bubbles. The material was run through a UV curing system (Fusion UV Systems, Inc.) at 4 ft/min with each side of gel exposed to UV lamp once.

### Example 16

### Formation of Tecophilic Polyurethane with IRP69-Ag

Tecophilic TG-500 polyurethane (The Lubrizol Corporation, Wickliffe, Ohio 44092) with IRP69-Ag 3.01 g Tecophilic SP-80A-150 was dissolved in 38.547 mL CHCl3 on roller mill, 19.995 g solution removed (1.0 g Tecophilic) and mixed with 0.0528 g IRP69-Ag by hand, poured on release liner to allow CHCl3 to evaporate. Cure was not inhibited.

### Example 17

### Formation of Tecoflex Polyurethane with IRP69-Ag

Test articles were prepared using Tecoflex EG-80A (The Lubrizol Corporation, Wickliffe, Ohio 44092) polyurethane with IMS additives, Ag-SCER and BA-SCER. Tecoflex MG-8020 was dissolved in CHCl3 on a roller mill, mixed with either IMS additive Ag-SCER or BA-SCER by hand. The mixture was then poured on a release liner to allow CHCl3 to evaporate. Cure was not inhibited. Samples were evaluated per ASTM E2180.

TABLE
Log10 Reductions of Bacteria by Antimicrobial Resins incorporated
into Polyurethane

| IASIS | | | | P. |
|---|---|---|---|---|
| Active | % Active Load | S. aureus | E. coli | aeruginosa |
| Ag- | 0.50% | 5.90 | 4.95 | 6.26 |
| SCER | | | | |
| Ag- | 1.00% | 6.06 | 4.95 | 7.00 |
| SCER | | | | |
| Ag- | 2.00% | | 4.68 | |
| SCER | | | | |
| BA- | 4.00% | | 4.13 | |
| SCER | | | | |

### Example 18

### Formation of Silicone Gel with IRP64-Ag

MED 6345 Silicone Gel (Nusil Silicone Technology, Carpinteria CA 93013) with IRP64-Ag 12 g part A+12 g part B+2.67 g IRP64-Ag (10% w/w) was mixed in Speedmixer, poured into mold and air bubbles allowed to escape (~20 min) and cured at 70° C. for 1 hr. Cure was not inhibited.

### Example 19

### Formation of Silicone Gel with IRP64-Benzalkonium

MED 6345 silicone gel (Nusil Silicone Technology, Carpinteria CA 93013) with IRP64-benzalkonium 3.07 g part A+3.07 g part B+0.323 g IRP64-BA (5% w/w) mixed by hand, poured onto release liner and air bubbles allowed to escape (~20 min) and cured at 84° C. for 23 min. Cure was complete and not inhibited by the ammonium compound.

### Example 20

### Formation of Silicone Gel with IRP64-Cetylpyridinium

MED 6345 Silicone Gel (Nusil Silicone Technology, Carpinteria CA 93013) with IRP64-cetylpyridinium 3.00 g part A+3.00 g part B+0.3305 g IRP64-BA (5% w/w) was mixed by hand, poured onto release liner and air bubbles allowed to escape (~20 min) and cured at 84° C. for 23 min. Cure was complete and not inhibited by the ammonium compound.

### Example 21

### Formation of Silicone Gel with IRP64-Octenidine

MED 6345 Silicone Gel (Nusil Silicone Technology, Carpinteria CA 93013) with IRP64-octenidine 3.060 g part A+3.060 g part B+0.3255 g IRP64-BA (5% w/w) was mixed by hand, poured onto release liner and air bubbles allowed to escape (~20 min) and cured at 84° C. for 50 min. Cure was complete and not inhibited by the ammonium compound.

### Example 22

### Formation of Liquid Silicone Rubber with IRP64-Ag

MED-4955 liquid silicone rubber (Nusil Silicone Technology, Carpinteria CA 93013) with IRP64-Ag 7.9 g part A+7.9 g part B+0.4997 g IRP64-Ag (3% w/w) was mixed in Speedmixer, spread on release liner and placed in vacuum oven at room temperature to remove air bubbles. Another release liner was placed on top, rolled with a jar to flatten and then cured (~80° C., ~10 min). Cure was complete.

### Example 23

### Formation of Liquid Silicone Rubber with IRP64-BA

MED-4955 liquid silicone rubber (Nusil Silicone Technology, Carpinteria CA 93013) with IRP64-BA 18.2 g part A+18.2 g part B was mixed in Speedmixer, 6.214 g removed and 0.3202 g IRP64-BA (5% w/w) mixed in by hand, spread on release liner and placed in vacuum oven at room temperature to remove air bubbles. Another release liner was placed on top, rolled with a jar to flatten and then cured (~80° C., ~10 min). Cure was complete and not inhibited by the ammonium compound.

### Example 24

### Formation of Liquid Silicone Rubber with IRP64-CP

MED-4955 liquid silicone rubber (Nusil Silicone Technology, Carpinteria CA 93013) with IRP64-CP 9.1 g part A+9.1 g part B mixed in Speedmixer, 6.195 g removed and 0.3208 g IRP64-CP (5% w/w) mixed in by hand, spread on release liner and placed in vacuum oven at room temperature to remove air bubbles. Another release liner was placed on top, rolled with a jar to flatten and then cured (~80° C., ~10 min). Cure was not inhibited.

### Example 25

### Formation of Liquid Silicone Rubber with IRP64-Oct

MED-4955 Liquid Silicone Rubber with IRP64-Oct 12 g part A+12 g part B was mixed in Speedmixer, 6.041 g removed and 0.318 g IRP64-Oct (5% w/w) mixed in by hand, spread on release liner and placed in vacuum oven at room temperature to remove air bubbles. Another release liner was placed on top, rolled with a jar to flatten and then cured (~80° C., ~10 min). Cure was not inhibited.

### Example 26

### Formation of UV-Curing Liquid Silicone Rubber with IRP64-A

Momentive 2060B UV-curing liquid silicone rubber with IRP64-A 41.9 g part B+1.52 g catalyst was mixed in Speedmixer, 7.16 g removed and 0.3768 g IRP64-Ag (5% w/w) mixed in by hand, spread on release liner and placed in vacuum oven at room temperature to remove air bubbles. The material was passed through UV curing system (Fusion UV Systems, Inc.) at 4 ft/min; each side of gel exposed to UV lamp once. Cure was not inhibited.

### Example 27

### Formation of UV-Curing Liquid Silicone Rubber with IRP64-BA

Momentive 2060B UV-curing liquid silicone rubber with IRP64-BA 41.9 g part B+1.52 g catalyst was mixed in Speedmixer, 7.28 g removed and 0.383 g IRP64-BA (5% w/w) mixed in by hand, spread on release liner and placed in vacuum oven at room temperature to remove air bubbles. The material was passed through a UV curing system (Fusion UV Systems, Inc.) at 4 ft/min with each side of gel exposed to UV lamp once. Cure was not inhibited.

### Example 28

### Formation of UV-Curing Liquid Silicone Rubber with IRP64-CP

Momentive 2060B UV-curing liquid silicone rubber with IRP64-CP 41.9 g part B+1.52 g catalyst was mixed in Speedmixer, 7.11 g removed and 0.374 g IRP64-Ag (5% w/w) mixed in by hand, spread on release liner and placed in vacuum oven at room temperature to remove air bubbles. The material was run through a UV curing system (Fusion UV Systems, Inc.) at 4 ft/min, with 4 passes required before gel could be removed and turned over, then one more pass on the reverse side. Cure was not inhibited.

### Example 29

### Formation of UV-Curing Liquid Silicone Rubber with IRP64-Octenedine

Momentive 2060B UV-curing liquid silicone rubber with IRP64-octenedine 15 g part B+0.544 g catalyst was mixed in Speedmixer, 6.3327 g removed and 0.3333 g IRP64-Oct (5% w/w) mixed in by hand, spread on release liner and placed in vacuum oven at room temperature to remove air bubbles. The material was passed through a UV curing system (Fusion UV Systems, Inc.) at 4 ft/min with each side exposed to UV lamp once.

### Example 30

### Formation of Tecophilic Polyurethane with IRP64-Ag

MED-4950 Tecophilic TG-500 Polyurethane with IRP64-Ag 3.07 g Tecophilic SP-80A-150 was dissolved in 38 mL CHCl3 on a roller mill. Subsequently, 19.975 g of the lacquer was removed from the container (equating to a solids content of 1.0 g Tecophilic TG-500) and the lacquer was combined with 0.0589 g IRP64-Ag and the mixture stirred by hand. The mixture was subsequently dispersed onto release liner and the CHCl₃ allowed to evaporate. The resulting film was durable, cosmetically acceptable, and demonstrated efficacy against several bacteria using a Kirby-Bauer disk diffusion assay.

### Example 31

### Formation of Tecoflex Polyurethane with IRP64-Ag

Tecoflex EG-80A Polyurethane with IRP64-Ag 5.012 g Tecoflex MG-8020 dissolved into 64.209 mL CHCl3 on a roller mill. Subsequently, 20.08 g of the lacquer was removed from the container (equating to a solids content of 1.0 g Tecoflex MG-8020) and the lacquer was combined with 0.0528 g IRP64-Ag g IRP64-Ag and the mixture stirred by hand. The mixture was subsequently dispersed onto release liner and the CHCl3 allowed to evaporate. The resulting film was durable, cosmetically acceptable, and demonstrated efficacy against several bacteria using a Kirby-Bauer disk diffusion assay.

### Example 32

### Preparation of IRP69-Cu-Modified Glidden Duo Premium Paint+Primer

IRP69-Cu (1-10 μm) was blended into acrylic latex enamel paint (Glidden Duo) at concentrations ranging from 0.5-2.0 wt %. The fluid compositions were painted (brushed and rolled) onto Leneta scrub test panels and the paint allowed to dry. Following extraction in deionized water, the painted samples were tested by ASTM E2180 to determine the efficacy of the Cu(II) modified acrylic latex enamel. As compared to unmodified control coated and extracted test panels, at 1.5 wt % loading the copper-modified paint demonstrated >5-log reductions against drug-resistant forms of Staphylococcus aureus, Enterococcus faecalis, Pseudomonas aeruginosa, Acinetobacter baumanii, Klebsiella pneumoniae, and Escherichia coli.

### Example 33

### Preparation of MAC-3-Ag

10.29 g of Dowex MAC-3 (The Dow Chemical Company, Liquid Separations, Midland, MI 48641-1206) material was slurried into 150 mL of 0.1N NaOH solution for 20 minutes and the MAC-3 filtered rinsed until the filtrate was neutral pH and the solid slurried in 150 cc deionized water and 8.5 g AgNO₃ (Note: 3.8 mEq/g requires 6.64 g to yield 100% of the exchange capacity) was (Fluka) added and the mixture stirred for 1.5 hours in the absence of light. The material was filtered, washed and dried under vacuum at 130° C. and the mass balance determined (13.45 g). This represents an increase in mass of 3.16 grams represents approximately a yield of 56%.

### Example 34

### Preparation of MED-4955 Liquid Silicone Rubber with MAC-3-Ag

8 g part A+8 g part B+0.5105 g MAC-3-Ag (3% w/w) mixed in Speedmixer, spread on release liner and placed in vacuum oven at room temperature to remove air bubbles. Another release liner was placed on top, rolled with a jar to flatten and then cured (~80° C., ~10 min). Cure was complete.

### Example 35

### Preparation of MAC-3-Cu and Amberlite IRP64-Cu

Dowex MAC-3 (The Dow Chemical Company, Liquid Separations, Midland, MI 48641-1206) or Amberlite IRP64 (Rohm and Haas Company, a subsidiary of Dow Chemical Company, Philadelphia, PA 19106-2399) weak cation-exchange material (Na+ form) was stirred in a minimal amount of deionized water and a large excess (~10-500 molar excess) of copper sulfate (Cu(SO4)2), copper acetate, or copper chloride and the mixture stirred at 80° C. for 1 hour using a mechanical stirrer. The (blue-colored) salt was filtered and rinsed with deionized water until the filtrate was clear (no blue). The salt was dried at 130° C. under vacuum, milled dry in a stainless-steel vessel and used in the formulation of silicone rubber materials. Neither MAC-3-Cu nor Amberlite IRP64-Cu inhibited the cure of silicone elastomers (Momentive Performance Materials UV-curing silicone (2060) or Nusil MED-4955. Elemental analysis (Cu) by ICP-OES demonstrated copper loading from 20-27 wt %.

### Example 36

### Preparation of MAC-3-Cu-Modified Glidden Duo Premium Paint+Primer

MAC-3-Cu as above as a finely divided powder was added to Glidden Duo at levels to yield dry paints with Cu concentrations in the 0.5-2.0 wt % range. The paint remained fluid with mixing and did not result in precipitation of the paint formulation. The paint was easily applied to varieties of surfaces and with drying a very light (baby blue) coating resulted. When coated onto a Leneta test panel, and the panel extracted to remove surfactants the surfaces of ≥1.0 wt % demonstrated broad spectrum antimicrobial effectiveness against an array of gram positive and gram-negative organisms as determined using the ASTM E2180 assay method.

### Example 37

### Antimicrobial Activity of Silicones, Rubbers and Other Polymer Composites Containing Activated Ion-Exchange Polymer Salts

Results demonstrate that silicone and other composite materials fabricated to include the active fine particulate polymer salts as additives demonstrate surprisingly potent antimicrobial effectiveness, for example as demonstrated by zones of inhibition in Kirby-Bauer disk diffusion assays.

### Example 38

### Preparation of Polymer Salts Using Crosslinked Polycarboxylated Weak Cation-Exchange Materials (General Procedure)

Amberlite IRP64 and MAC-3 weak cation-exchange material was stirred in a minimal amount of deionized water and a large excess (~10-500 molar excess) of the salt (containing the biologically active exchange cation of interest, such as benzalkonium chloride) was added and the mixture stirred by the addition of a mechanical stirrer for 60 minutes. The solid was filtered washed with copious amounts of deionized water (until the filtrate does not contain any of the active ionic agent (benzalkonium chloride for example) as evidence from ultraviolet spectroscopic evaluation of the filtrate. The modified IRP64/MAC-3 was dried under vacuum at 130° C. and the material was milled with the aid of an IKA homogenizer and the milled particulate polymer salt put through a sieve with a 35 μm cutoff. The powder was dried under vacuum and used for addition to various polymer composite formulations.

### Example 39

### Preparation of Amberlyst A21-Acetylsalicylic Acid (ASA)

Amberlyst A21 (4.6 mEq/g exchange capacity) and acetyl salicylic acid (ASA) were stirred together at room temperature in a solution of water/isopropyl alcohol (2:1) for 12 hours and the product filtered, washed with water and soxhlet extracted with isopropyl alcohol (12 hours). The product (Amberlyst A21-ASA) was air and vacuum dried, and the yield determined to be 30% of theoretical exchange capacity) milled to 5 μm particle size and incorporated into LSR silicone rubbers Performance Materials UV-curing silicone (2060) and Nusil MED-4955 (UV curing and thermal curing materials respectively). Both materials cured as expected and the release of ASA from the silicone materials was observed and monitored by UV spectroscopy with exposure of the material to PBS solution. Similarly, ASA was associated with the strongly basic anion-exchange resin AMBERLITE FPA40-CI (exchange capacity >1 mEq/g), a food grade strong base anion-exchanger (a polyamine/polyammonium salt. This resin, a polyaminated ion-exchange material demonstrated effective binding (~1.0 mEq/g) of ASA and similarly binding and releasing dexamethasone sodium phosphate (DexSP) anion.

Under mild conditions room temperature aqueous ethanol, ASA can be bound to Amberlyst A21 to yield an ion-exchange material including about 30% of the theoretical exchange capacity of the material (4.6 mEq/gram). Following soxhlet extraction (isopropanol) and drying, the material was incorporated into Nusil MED 4950 (NuSil Technology LLC, 1050 Cindy Lane, Carpinteria, CA 93013) and the cure of the silicone proceeded uninhibited. ASA is readily released from the resulting composition intact, as determined by UV spectroscopy. ASA cannot be incorporated as the free acid as it is not stable once heated in the curing process. This example is another representation of the stability imparted as a consequence of binding an organic molecule to an organic ion exchange backbone. The above exemplary, antimicrobially active polymer composites all exhibit high levels of antimicrobial activity according to the various assays for bactericidal, bacteriostatic, and inhibition of bacterial transfer contamination risk described herein.

### Example 40

### Incorporation of Amberlyst A21-ASA into Silicone Rubber

Amberlyst A21-ASA was successfully incorporated into Momentive Performance Materials 2060 UV-curing LSR without any inhibition of cure.

### Example 41

### Incorporation of Chlorhexidine in Silicone Rubber

Chlorhexidine (CHX), a molecule that is susceptible to thermal degradation to yield the carcinogen, p-chloroaniline, above 70° C. is stabilized when bound to the ion-exchange material (polystyrene sulfonate (PSS) as well the crosslinked version IRP69). In binding the active antiseptic to an organic ion exchange material (IRP64) using the reaction of IRP64-H (acid form) with chlorhexidine diacetate. The yield of IRP64-chlorhexidine is approximately 80% of the theoretical exchange capacity of 10 mEq/g (5 mEq/g for a dication such as chlorhexidine). Once the resin is thoroughly dried, it can be readily milled to submicron size where within the jar the temperature can reach approximately 80° C. Under these circumstances, in the presence of water, the risk of hydrolysis to yield p-chloroaniline increases substantially.

### Example 42

### Octenidine Hydrochloride

Octenidine hydrochloride has been observed to inhibit the cure of thermal and UV-curing silicone rubber materials at levels of less than 2 wt % loading. As such, it is of little utility to be included into a silicone material. In addition to the inhibition of cure, such an approach can lead to porosity once the compound has eluted from its matrix. However, the binding of octenidine to IRP64 by the reaction of IRP64-Na with octenidine dihydrochloride. In this reaction, approximately 3.0 mEq/g of the dicationic octenidine (6.0 mEq of IRP64 sites). The material was milled to 1-10-micron particle size and incorporated into silicone rubber as high as 5.0 wt. % without any inhibition of thermal and UV curing silicone rubber.

### Example 43

### Preparation of Copper Cellulose Phosphate

Copper cellulose phosphate can be prepared by exposing sodium cellulose phosphate to an excess of copper (II) sulfate in deionized water, filtering and washing the solid until no residual copper (II) sulfate is detected in the filtrate. Similarly, cellulose phosphate (acid form) can be used in conjunction with copper (II) acetate to yield the cellulose phosphate copper salt and acetic acid. Cellulose phosphate materials derivatized to include metal ions such as copper may be provided as additives for the manufacture of articles to include drywall construction material for example.

Strong and weak cation-exchange resins modified to incorporate Cu(II) can be incorporated into polymeric materials to render the surfaces effective against bacteria, viruses, and fungi for example. In the case of a polymer matrix surface, to include laminate materials, incorporating Cu(II) or Fe(II) modified ion-exchange resins, the use of hydrogen peroxide solution with saline or HEPES buffer (Fenton or modified-Fenton reaction) can be used to aid in the disinfection of such surfaces. The addition of peroxide to surfaces comprising metal ion-modified ion-exchange resins can result in the formation of free radical species (hydroxyl radicals) that can be efficient at killing microbial and viral pathogens.

### Example 44

### Antimicrobial Efficacy

Experiments that have evaluated the antimicrobial capability of Dow Chemical Dowex MAC-3 (The Dow Chemical Company, Midland, Michigan 48674) weak cation-exchange material and Amberlite IRP64 (Rohm and Haas Company, a subsidiary of Dow Chemical Company, Philadelphia, PA 19106-2399) weak cation-exchange material in Cu(II) and Ag(I) forms were shown to have significant zones of inhibition in Kirby-Bauer assays and the zones were determined to be similar to those of strong cation-exchange materials such as Amberlite IRP69 modified to include the same cations of (Cu(II) and Ag(I)) when tested against Staphylococcus epidermidis, Staphylococcus aureus, Pseudomonas aeruginosa, and Enterococcus faecalis.

### Example 45

### Incorporation of Activated Ion-Exchange Polymer Salts into Silicone Rubber

Dowex Mac-3 and IRP64 weak cation (polycarboxylate) exchange resins were modified to include silver ion (See, for example published US patent application No. US20100247544A1 entitled “Compositions and Methods for Promoting the Healing of Tissue of Multicellular Organisms” and published Sep. 30, 2010, the entirety of which is incorporated by reference herein). This silver Mac-3 can be dried under vacuum (135° C.) to yield an off-white solid that was ground to particles and sieved with a 35 μm cutoff sieve. The particles can be dried again under vacuum and formulated into two silicone materials and these materials (silicones with Ag-Mac-3 and the Ag-Mac-3 alone as particles) were evaluated using a Kirby-Bauer assay. Amberlite IRP64 was treated with 0.1N NaOH solution and the sodium salt (Amberlite IRP-64 (Na+)) was filtered and washed with deionized water until the pH of the filtrate was neutral.

The salt is used in alternate examples to prepare Ag+, Cu++, benzalkonium+, chlorhexidine++, octenidine++, doxycycline+, minocycline+, as well as mixed ion material salts, such as materials incorporating silver and copper, silver and zinc, or copper and zinc ions simultaneously. As salts, these particles were incorporated into LSR silicone rubber materials at 5 and 10% loading w/w. Silicones and polyurethanes including various additives comprising a variety of metal and organic ions have been prepared at loading of up to 50 wt %. It is feasible to incorporate the active fine particulate polymer salts as additives in composite mixtures as described herein at levels greater than 25 wt %, however loadings between 1 wt % to 10 wt % appear to be highly active for most materials and uses.

### Example 46

### Platinum Catalyzed Silicone Rubber with Ammonium Salts

Previous attempts to cure platinum catalyzed silicone rubber formulations compounded with benzalkonium chloride, cetyl pyridinium chloride and other ammonium salts have met with failure. The two-part rubber remained liquid-like of petrolatum consistency. In addition, when curing thermal silicone systems, temperatures of 150° C. are required for periods of generally at least five minutes. Because benzalkonium chloride melts at 35° C., the salt, if dispersed as a particle will become molten during the attempt to cure the polymer thus causing the compound to flow/ooze from the material.

However, upon exchange of chloride for polysulfonated anions such as for strong cation-exchange materials including non-crosslinked polystyrene sulfonate and crosslinked polystyrene sulfonate such as Amberlite IRP69 or crosslinked carboxylated weak cation-exchangers such as Amberlite IRP64, the silicone rubber materials (Nusil MED 4950 thermal curing system and the UV curing system, i.e. Momentive Performance Materials 2060 UV-curing liquid silicone rubber or LSR), demonstrated full cure under normal curing conditions without any melting of the added material and resulted in the absence of any voids in the material. This unexpected result provides for the preparation of silicone rubber materials that demonstrate zones of inhibition against bacterial species amenable for medical uses to combat bacterial infection and surface transmission. The effectiveness of several 5% Amberlite IRP69-Benzalkonium (Amb-BA), Amberlite IRP69-cetyl pyridinium (69-CP), and linear (water-soluble) polystyrene sulfonate salts of benzalkonium (PSS-BA) and cetylpyridinium (PSS-CP) composited with silicone rubbers (Nusil MED 4955 and Momentive 2060 LSR) against Staphylococcus aureus and Enterococcus faecalis.

This demonstrates activation of silicon polymer composites using ammonium polymer salts using a (platinum) curing silicone system with a non-crosslinked strong cation-exchanger (PSS), further expanding the compositions and uses provided here for constructing ion-exchange polymer salt composites with silicone and other thermoset and thermoplastic polymers. These various exemplary composites were also effective against Staphylococcus epidermidis and to a lesser degree against the Gram-negative Pseudomonas aeruginosa. Effective milling/particle size reduction has been demonstrated for a variety of commercially available resins, including IRP69-Na through two consecutive milling steps where, A) is Poly(Sulfonated Styrene-divinylbenzene) IRP69-Na (Rohm & Haas) as received, B) post milling with 5 mm stainless steel media in heptane non-solvent, C) post milling with 0.5 mm zirconia media in heptane non-solvent. With each milling step the size distribution becomes more refined around the median value. As received, IRP69-Na size distribution spans approximately 10-1400 μm (Δ=1300 μm). Following the first milling step the range spans approximately 0.8-10 μm (Δ=9.2 μm), and after the second milling step the distribution spans approximately 0.1-0.8 μm (Δ=0.7 μm). The first milling step utilizes stainless steel and the non-solvent medium heptane and in the second milling step zirconia ceramic is utilized with heptane non-solvent.

Demonstration of effective particle size reduction in a single (dry milling) step has been demonstrated for a variety of resin derivatives in stainless-steel with ceramic milling jars and media.

### Example 47

### Analysis of Surface Characteristics and Bacterial Adhesion to Extruded Silicone Rod Incorporating 2 wt % IRP69-Ag

Data shows that extruded Dow Corning Q7-4750 formulated to include 2 wt % IRP69-Ag silicone possesses overtly active surfaces as determined by a bacterial adhesion/proliferation assay and subsequent image analyses of the surfaces. Field emission scanning electron micrographs (FE-SEM) and silicone samples containing Ag-SCER (500 nm mean particle size) at 2% w/w reveal no surface irregularities after being challenged with 10⁵ CFU of E. coli for 24 hours. (D) Silicone slab sample of IR69F-Ag at 2% w/w showing no surface irregularities after being challenged for 4 hours with 10° CFU of S. aureus. Following exposure, a small number of bacterial colonies are observed on the surface. These cells are rounded and appear dead.

### Example 48

### Adhesion Assays

Formulation of silicone rubber samples of Dow Corning Q7-4750 formulated to include 2 wt % IRP69 modified with Copper (Cu), Benzalkonium, Silver (Ag), and Ag/Cu, as well as a blank unmodified silicone sample following exposure to 10° CFUs of E. coli and cultured in tryptic soy broth at 37° C. for 18 hours at which time the samples were lightly rinsed with phosphate buffered saline (PBS) PBS to remove the loosely adhered bacteria and subsequently sonicated in PBS to remove adherent cells. Serial dilutions of the sonicated samples were made prior to plating on standard plate count agar. The data reveal that the modified surfaces showed very little reduction in bacterial adhesion when compared to the controls.

### Example 49

### Biofilm Formation Assay

By way of comparing the number of colony forming units (CFUs) per mm² identified on the surfaces of untreated Dow Corning Q7-4750 silicone (Blank) and Dow Corning Q7-4750 modified to include Cu, Ag, Ag/Cu, and benzalkonium-modified IRP69 (all at 2 wt %) with exposure to 10⁵ CFUs of E. coli (cultured in tryptic soy broth at 37° C., 36 hrs.), no difference in bacterial adhesion was observed in early time points (3 hrs.) vs. controls. The assay demonstrated that reduced biofilm formation results on the composite surfaces when compared to the unmodified silicone Q7-4750.

### Example 50

### Time-to-Kill Assays for Ag-SCER-Modified Silicone Rubber (2.0 wt %) vs. Staphylococcus Aureus

Following an inoculum of 10° C. FU/mL of E. coli in synthetic urine (recipe), composite and control samples were incubated at 37° C. and at time points of 0, 3, 8, 16, 24, and 32 hours samples were removed from test and adherent bacteria removed and counts determined. The data reveal that after 3 hours a one-log reduction is observed and after 32 hours a 6-log reduction is evident. These data may indicate that a surface such as this will have a significant positive impact on the likelihood of bacterial colonization of the inner lumen of urinary catheters where protein is absent except in some extremely sick, renal-compromised patients.

### Example 51

### Antimicrobial Sulfonated Polystyrene-Co-Divinylbenzene (IRP 69)

Data reveals that the surfaces of silicone rubber materials (Q7-4750) modified to include 2.0 wt % of IRP69-Ag, IRP69-benzalkonium, IRP69-Cu, and binary formulations to include 1.0 wt % of each of IRP69-Cu/IRP69-benzalkonium and IRP69-Ag/IRP69-benzalkonium are highly effective at reducing surface (Staphylococcus aureus) bacterial counts even following pretreatment of the surfaces with fetal bovine serum indicating that protein exposure does not limit material efficacy.

### Example 52

### Milling of Ion-Exchange Materials

One exemplary high energy milling process for use within the invention utilizes planetary ball milling in a ceramic (zirconium) lined stainless steel milling vessel. Zirconia milling media (3.0 mm) are added into the chamber to occupy approximately ⅔ of the bulk chamber volume. Approximately ⅓ of the bulk volume is occupied by any of the porous active ion-exchange polymer salt particulate material. A non-solvent liquid is then added in an amount approximately equal to ⅓ of the container bulk volume (typically the non-solvent is added so as to percolate into and fill void spaces between milling media and active polymer salt particles, and to fill void, pore and channel spaces within the porous polymer salt particles. The non-solvent liquid may comprise a heptane non-solvent, or any other suitable non-solvent. Suitable non-solvents more generally can include, for example, intermediate or high boiling point alkanes, exemplified by heptane or mixtures of heptanes, octane, isooctane (2,2,4-trimethylpentane), petroleum distillates (high boiling Pet ether). Lower boiling solvents such as hexane can be used, however this may raise the risk of fire or explosion.

Within the instant example, the milling vessel was topped off with non-solvent, sealed then placed (clamped) into a PM100CM planetary ball mill. The sample milled for approximately 2 hours at 500 rpm. After this milling was stopped (more generally, when a desired milled particle size and uniformity are obtained), the fine particulate ion-exchange polymer salt is separated from the non-solvent (e.g., by evaporation) and media (e.g., by sieving).

In other working examples, a second stage of milling was conducted, wherein the active ion-exchange polymer salt particles were second stage-milled using smaller zirconia milling media (0.5 mm).

According to this exemplary two stage milling process, particle size (alternatively expressed as average or median diameter) and size variation for the fine particulate biologically active ion-exchange polymer salt materials were shown to be within a predicted, desired size range and to have a predicted, desired particle size uniformity. Briefly, particles from the second stage of milling exhibited particle sizes and uniformity measured at approximately 500 nm average diameter with standard deviations of approximately ±0.75 μm, in other examples approximately ±0.50 μm, and in other examples about ±0.25 μm. In yet other working examples, milling of various modified resins to distributions of 1-10 μm were achieved using stainless grinding media (5.0 mm) in one hour. The resulting material was sieved to remove the grinding media and subsequently sieved to a fine powdered material.

Temperature of milling is an optional control condition that can yield improved milling results in certain embodiments. In demonstration milling runs, excellent milling results were obtained as described above when the temperature of the milling vessel and contents was maintained, for at least a portion of a milling cycle (measured using an IR thermometer), at approximately 80-85° C.

This elevated, controlled temperature imparted to the milling chamber and contents elevates pressure within the sealed vessel chamber and lowers viscosity if the milling milieu (non-solvent, milling media and active ion-exchange polymer salt material) improved milling outcomes for some samples compared to results observed at lower milling temperatures.

### Example 53

### Surface Characteristics and Stability of Activated and Partially Discharged and Environmentally Exposed Polymer Composites

The use of fine particulate active ion-exchange polymer salt materials in formation of composites (by combining the active polymer salt particles with polymer precursors to form solid composites) yields polymer composites of the invention having surprisingly uniform and smooth surface properties free of voids and cracks or other surface defects. Additionally, these biologically active polymer composites retain their distinctly smooth and unmarred surface character even after exposure to aging and exposure to photodegradative, thermal degradative, microbial degradative, and chemically transforming (e.g., ionizing, oxidizing, hydrolyzing) environmental conditions. Demonstrating exemplary surface characteristics and performance of these inventive composites, Field emission scanning electron micrographic (SEM) images of: silicone rod samples containing IR69F-Ag at 2% silver loading (w/w), magnification 2000× (Panel A), and Silicone rod samples containing IR69F-Ag at 2% (w/w), magnification 7000× (Panel B) revealed very smooth defect free surfaces. These samples were challenged, in 24-well plate format, with 1.0 mL of 10⁹ CFU/mL of E. coli (Panel C) added to a well containing 1.0 mL of tryptic soy broth and the samples exposed for 24 hours at 37° C. In similar Fashion, a sample (Panel D) was challenged, in 24-well plate format, with 1.0 mL of 10⁸ CFU/mL of S. Aureus added to a well containing 1.0 mL of tryptic soy broth and the samples exposed for 24 hours at 37° C. In these examples, the samples were exposed to microbial, and ionic factors that may be expected for the most challenging, practical microbial contamination conditions for a product to experience. As shown in Panels A-B, silicone slab samples of IR69F-Ag, at 2% (w/w) without any bacterial exposure (Panels A and B) and tubing (IR69F-Ag/2.0 wt %) following exposure to 10⁵ CFUs of E. coli and after 10⁸ CFUs S. Aureus. Samples were removed from the wells after 24 hours of exposure, lightly rinsed with deionized water, dehydrated by serial dilution with EtOH and subsequently fixed using formalin solution. As evident from the photomicrographs, the ionic/chemical challenges exhibited no detectable surface irregularities at magnification up to 7000×. These assays further demonstrate that, although bacteria do adhere to the composite surfaces (as indicated by arrows), they are not prevalent on the modified silicone surfaces, and exposures to both the bacteria and ionic and chemical degradative factors present in the experimental growth media do not appreciably alter the smooth, defect-free surface characteristics of the active polymer composites.

In more detailed embodiments, the surfaces of active polymer composites of the invention remain essentially free of surface irregularities and defects that could promote microbial colonization, under a range of storage and use conditions, for extended storage and use periods. Under various environmental challenges (photodegradative, thermal, chemical degradative, microbial degradative), the surfaces of active polymer composites remain free of cracks, pits, voids or other defects of sizes that could receive and shelter any microbial cell or colony. Expressed more distinctly, the active composites of the invention possess smooth surfaces essentially free of pits, voids or cracks. In certain embodiments, the surfaces of active polymer composites of the invention remain free of structural defects including voids, pits or cracks having a largest void (i.e., wall to wall, or floor to opening) dimension of 1-5 μm or less, often no larger than 500 nm, 400 nm, 200 nm or even smaller.

Activated polymer composite surfaces thus defined will have no more than 1-5 of these types of defects per square cm of surface area, and thus satisfy the definition of these polymer composites as having “microbially resistant” surface integrity (smooth, defect-free micro-texture).

Of additional surprising advantage, the active polymer composites of the invention retain their novel “microbial surface resistance” marked by a smooth, defect-free surface architecture even after extended periods of use and exposure to environmental degradative influences. This is shown here following prolonged exposure to combined ionic, chemical and microbial degradative effects. In one important aspect, the polymer composites retain their microbial resistant surface character even after prolonged exposure to ionizing solutions (e.g., microbial growth media). Such solutions cause ion-exchange that leaches or dissociates some of the biologically active counter-ions from the polymer composite surface. More specifically, counter-ions present in ionic solutions uncouple ionic salt associations of the biologically active counter-ions with ion-exchange groups on the functionalized ion-exchange polymer salt (incorporated in fine particulate form in the polymer composite). This replaces some of the active counter-ions by salt exchange with new substitute counter-ions present in the ionic solution (e.g., Na+). This ionic degradative process is in fact a mechanism for “controlled activation and drug release” desired for some applications of the active polymer composites. In these uses, the composites not only function by way of surface active chemistry, but in contact with physiological fluids and tissue and other ionic media they are able to dissociate some of the biologically active ionic agent in soluble form to exert biological activity away from the polymer surface (e.g., in a wound environment, or target tissue or compartment proximal to the polymer surface and contactable by solubilized biologically active ionic agent).

Of significance, “ionic degradation” influences (exemplified by prolonged exposure to physiological or other ionic solutions for prolonged periods of 6-24 hours or more, one to several days or weeks, even 1-6 months or longer) do not substantially alter the microbially resistant surface texture of the active polymer composites. Despite the observed (and in many embodiments desired) mechanism and operation of “controlled activation and drug release” (discharging biologically active counter-ion from the exposed composite surface), the polymer composites do not lose their smooth surface architecture. They remain free of defects so as to retain “microbially surface resistance” (i.e., remain substantially free of defects large enough to provide anchorage or shelter for any microorganism or microorganism colony), despite this ionic degradation or discharge. In part, this is mediated by replacement of discharged, biologically active ionic agent on the polymer composite surface by counter-ions in the offending ionic medium, solution or tissue. (Ion) exchange leaves no detectable surface defects, due to the generally small size of original, biologically active counter-ions loaded within the polymer composite (which will generally be replaced by similar small physiological ions, such as Na+). In some embodiments, surface maintenance and restoration will involve “recharging” the polymer composite surface using a salt solution comprising the original biologically active counter-ion to replace discharged counter ions (either by salt exchange to replace substituted counter-ions, or by re-association of the biologically active counter-ion with a functional group on the ion-exchange polymer left vacant after counter-ion discharge). Alternatively, the surfaces can be recharged through the act of polishing the composite surface to reveal unaffected and fully charged particles able to exchange-release active cations into the environment. Notably, the studies here show that, despite prolonged exposure to ionic degradative factors, i.e. extraction, the biologically active polymer composites of the invention do not shed or dislodge fine particulate ion-exchange polymer salt particles (embedded in the composite or composite surface), despite the observed discharge of biologically active ionic agent from association with the polymer salt particles. Predictably, discharge by dissociation of a substantial portion of biologically active counter-ions from salt association with the fine polymer salt particles could diminish their size and structural integration within the polymer composite, allowing them to be shed, dislodged or otherwise disintegrated from the surface of the composite. Natural ionic replacement (and artificial “recharging” as described above) of the polymer salts by organic ion exchange in physiological and other ionic solutions surprisingly overcomes this problem. Unexpectedly, there is substantially no detectable loss of intact fine particulate ion-exchange polymer salt particles observed from polymer composite surfaces of the invention following prolonged exposure to ionic degradative factors as described. The surfaces of the polymer composites remain substantially free of defects (no greater than one defect per square centimeter of surface) of approximately equal or greater size than any of the fine particulate polymer salt materials employed (e.g., 200-500 nm, 500 nm-800 nm, 1-2 μm, 5-10 μm). This is also the case observed following prolonged storage and use of the subject polymer composites even under extreme conditions of thermal degradation (e.g., at temperatures above 200 degrees, 300 degrees, even 400 degrees for periods from one to several hours), photodegradation and chemical degradation. Within the instant example, the milling vessel was topped off with non-solvent sealed then placed (clamped) into a PM100CM planetary ball mill. The sample milled for approximately 2 hours at 500 rpm. After this milling was stopped (more generally, when a desired milled particle size and uniformity are obtained), the fine particulate ion-exchange polymer salt is separated from the non-solvent (e.g., by evaporation) and media (e.g., by sieving).

### Example 54

### Preparation of Epoxy Incorporating IRP69-Ag

EPO-TEK 301 [Epoxy Technologies] was formulated to prepare a total 9.5 grams of epoxy for cure (4.00 grams of A and 1.00 grams of B. 0.57 grams of IRP69-Ag (SULFONATED POLYSTYRENE-CO-DIVINYLBENZENE Ag) in a Speedmixer cup. The mixture was mixed to evenly disperse the composite blend and the mixture cured by heating to 80° C. The antimicrobial properties of the surface were evaluated using an ASTM E2180 method.

MAX CLEAR epoxy resin (Food Safe, FDA Compliant) was formulated in identical fashion to include IRP69-Ag and the resulting material coated onto a scrub test panel (Leneta) for evaluation by ASTM E2180.

### Example 55

### Preparation of Acrylic Incorporating IRP69-Ag (2.0 wt %)

Acrylic (SCIGRIP 40), a two-part compound was combined with IRP69-Ag (2.0 wt %). Handle with care and avoid the two components to come into contact with each other during the process. For this particular trial, IR69F-Ag was the additive utilized. IR69F is a crosslinked polymer of polystyrene sulfonate (PSS-DVB), with silver ion exchanged onto it. The resin used in this example was milled to approximately 400 nm. The acrylic material was evaluated against P. aeruginosa, E. coli, and S. aureus using an optimized (modified) ASTM E2180 assay (ASTM International, West Conshohocken, PA, 2007) and the results demonstrated significant knockdown of the referenced pathogens.

### Example 56

### Preparation of Polyurethane (Tecoflex) IRP69 Composite

Tecoflex EG-80A (9.8 grams) was dissolved into either THF or methylene chloride to about 25% solids and 0.20 grams of IRP69-Ag are added, and the mixture homogenized with stirring by hand. The solution was dispensed onto a glass plate and the solvent allowed to evaporate in a hood. The film was transferred to an oven set at 65° C. to completely remove residual solvent from the sample. The resulting material was a cosmetically acceptable tan color, maintained the characteristics of the parent polyurethane, and demonstrated antimicrobial effectiveness versus S. aureus, E. coli, and P. aeruginosa as determined from Kirby-Bauer disk diffusion assays.

### Example 57

### Microbial Self-Decontamination Surface Activity and Prevention of Contaminant Transfer Risk Potential by IRP69Ag-Silicone

The instant example demonstrates novel “self-decontaminating” surface activity of active polymer composites of the invention. Additionally, and by virtue of this novel surface-active property, the active polymer composite biomaterials provided herein secondarily function by reducing contaminant transfer risk in hospital, industrial and other environments. In exemplary hospital settings, traditional fomite surfaces made or coated with antimicrobially active polymer composites of the invention are “self-decontaminating”, in that the original polymer composite surface (or regenerated or recharged composite surface) effects potent antimicrobial (e.g., bactericidal and bacteriostatic) activity, by killing microbes in prolonged contact (sufficient for surface activity expression) with the composite surface, and also through microbiostasis (without destroying or killing the microbe, rendering it functionally static as marked by an inability to colonize another surface or subject and survive or proliferate new microbes). In this exemplary study, the ability of E. coli to adhere to and persist on an exemplary extruded silicone rod (0.008 in. OD, Helix Medical Inc., Carpinteria CA) containing 2.0 wt % IRP69-Ag was tested for purposes of determining both contamination “resistance” of the composite surface, and its self-decontaminating activity. This assay in certain constructions also provides “time of kill” values for determining bactericidal activity (by providing values for how long test bacteria can remain adhered to the composite surface before dying). From silicone slabs fabricated to include 2.0 wt % antimicrobially active fine particulate ion-exchange polymer salts, disks were cut using a punch (hole) die, mm). The silicone “punch-outs” comprising the active fine particulate ion-exchange polymer salts are exposed to 10⁸ cfu/mL of bacteria to determine ability of the bacteria to adhere to the surfaces over time (as compared to non-active silicone controls without antimicrobially active fine particulate ion-exchange polymer salt added).

### Example 58

### Discoloration Reversal Process for Novel Silicone Polymers Containing Oligodynamic Metal as Biologically Active Ionic Agent

Exemplary active polymer composites of the invention incorporate an oligodynamic metal such as silver as the biologically active ionic agent integrated within the composite through salt association with fine particulate ion-exchange polymer resins, i.e. addition onto the backbone of the resin via ion exchange to create a biologically active polymer salt. These active silicone rubber composites can be readily extruded (e.g., they have excellent green strength) to yield uniform tubing or other biomaterials, sheets, films and components. Upon standard curing of these and other, related biomaterials, it was observed that the silicone/metal composites develop darkened, reddish coloration characteristics. These cure-darkened color features are undesirable for many consumer, industrial and clinical uses. In particular they are simply less aesthetically pleasing in consumer contexts, and more so in clinical and industrial applications. As many of the uses contemplated for these materials relate to hygiene, where light coloration of materials is much preferred. Light coloration further enhances ability to detect soiling, surface defects, and contaminants (e.g., body fluids, caustic or toxic contaminants, etc.) The instant example details an important discovery for providing novel products and methods featuring lightened or non-discolored, biologically active silicone/metal composite materials (actually lightened by reversal of cure-mediated discoloration. The silicone polymer composite containing silver (2.0 wt. % IRP69-Ag in Dow Corning Q7-4750, as fabricated by Helix Medical for Vachon) was cured for a standard curing period of 5-10 minutes at 150° C. This standard curing process yielded a darkly discolored, conventionally cured silicone composite material. Surprisingly, this pronounced discoloration was discovered to be reversible following alternate methods of extended or elevated temperature curing. In this example, the silver-silicone composite material was post-cured for an extended period of 1-2 hours at 150° C., during which extended curing the silicone-silver composite material lightened to a much more desired manila color.

### Example 59

### Activation of Chargeable Polymer Composite Surfaces by Post-Fabrication Surface Treatment

IRP69 (acid form, —SO3H) was placed into DI water and stirred. 4.6 mEq/gram of Fe(OAc)₂ was added to the mixture (note: Fe(II) necessitates the use of ½ the molar amount given the divalent nature of iron. The reaction was allowed to stir for 1-2 hours at room temperature and the presence of acetic acid (HOAc) was noted.

The resulting resin (IRP69-Fe) is filtered, washed and dried. Milled to 1-10 μm and incorporated into silicone at 1-5 wt %. The silicone is exposed to saline+hydrogen peroxide 3% (non-stabilized) in the presence of methylene blue and the observation of a decolorizing from blue to gray is indicative of the formation of superoxide.

This novel “Fenton Chemistry” charging reaction to yield potently antimicrobial hydroxyl radicals at the surfaces of polymer composites (through manual post-fabrication activation, involving “surface activation” or “surface charging” by exposure of the composite surface to a peroxide or other charging chemical or solution (to generate a new, biologically active chemical by-product at the active or charged surface of the polymer composite), provides yet another conceptual breakthrough in the fields of biomaterials production and application. Comparable results were obtained with another polymer composite of the invention incorporating an iron additive. The colorimetric results were readily observed. As exemplified by the Fenton Chemistry model for surface activation, surface charging of the foregoing exemplary polymer composites occurs when a divalent metal ion, typically iron, is exposed to peroxide, leading to the formation of a radical species (e.g., hydroxyl radical (HO·)). Reactive oxygen species (ROS) have strong antimicrobial properties, and thus their renewable production by surface activation here evinces one embodiment of a surface active or surface chargeable polymer composite. This activation potential is renewable in the sense that the activation can be repeated for the same polymer composite surface, to yield multiple rounds of activation (e.g., successive events of ROS production at the polymer composite surface, manually controlled by simply spraying or wiping the surface with an activating solution). These exemplary results for hydroxyl radical were easily observed with methylene blue bleaching Decolorization of the methylene blue solution was observed almost instantaneously, with full decolorization (bleaching) of the solution complete after 2 min. © Silicone test swatches containing the iron additive (2.0 wt %) were also evaluated by adding methylene blue solution (0.1 millimolar) on to the surface and then adding peroxide (300 μL of 30% non-stabilized peroxide). After 15 min, decolorizing of the solution (bleaching) above the test was observed in the presence of peroxide. It is important to note that this bleaching effect was not observed when peroxide+saline was added to resin in the acid and sodium forms (no Fe(II) or Cu(II)).

### Example 60

### Preparation and Testing of Hydrophilic Polyurethane Foam Incorporating 0.25 wt % IRP69-Ag

A 10-gram sample of IRP69-Ag (1-10 micron particle size) was provided to Rynel Inc. (Wicasset, ME) and approximately 100 grams of hydrophilic open cell hydrophilic polyurethane foam (SE-3) was provided for testing. Kirby-Bauer disk diffusion assays using a 6 mm punched disk against Pseudomonas aeruginosa (PA), Enterococcus faecalis (EF), and Staphylococcus aureus (SA). The samples demonstrated zones of 0.08 mm vs. PA, 0.08 mm vs. SA, and 0.08 mm vs. EF were recorded. Control foams (polyurethane only) showed no zones under the same conditions.

### Example 61

### Preparation and Testing of Hydrophilic Polyurethane Foam Incorporating 5 wt % IRP69-Benzalkonium

A 10-gram sample of IRP69-benzalkonium (1-10 micron particle size) was provided to Rynel Inc. (Wicasset, ME) and approximately 100 grams of hydrophilic open cell hydrophilic polyurethane foam (A4) was provided for testing. Kirby-Bauer disk diffusion assays using a 6 mm punched disk against Pseudomonas aeruginosa (PA), Enterococcus faecalis (EF), and Staphylococcus aureus (SA). The samples demonstrated zones of 0.00 mm vs. PA, 0.17 mm vs. SA, and 0.08 mm vs. EF were recorded. Control foams (polyurethane only) showed no zones under the same conditions.

### Example 62

### Preparation and Testing of Hydrophilic Polyurethane Foam Incorporating 0.50 wt % IRP69-Ag

A 10-gram sample of IRP69-Ag, (1-10) micron particle size, was combined with 199 total grams of Covestro FP505 polyurethane foam formulation and the material allowed to cure onto the surface of wax paper. Kirby-Bauer disk diffusion assays using a 6 mm punched disk against Pseudomonas aeruginosa (PA), Enterococcus faecalis (EF), and Staphylococcus aureus (SA). The samples demonstrated zones of 0.08 mm vs. PA, 0.08 mm vs. SA, and 0.08 mm vs. EF were recorded. Control foams (polyurethane only) showed no zones under the same conditions.

In another demonstration of the antimicrobial efficacy of the IRP69-Ag-modified foam material, the modified and control foams were punched to a disk format and inoculated with 10⁶ CFUs of either Staphylococcus aureus or Pseudomonas aeruginosa from a 1:20 dilution of an overnight culture (0.40-0.55 mL) in simulated wound fluid with a control foam receiving the same inoculum. The foam test articles were incubated overnight in an incubator (37° C., 5% CO₂, 95% RH) and each of the samples subsequently neutralized using Dey Engle broth with vortexing to recover colony forming units. The subsequent extracts are plated and counted to determine log reductions. The dressing demonstrated strong preservative capability with a demonstrated log reduction for each bacterium in excess of 5 logs.

In an analogous evaluation a foam dressing loaded at 0.25 wt % was evaluated in identical fashion and an identical log reduction for each of the bacteria were measured.

### Example 63

### Preparation of Polypropylene (PP) Incorporating 2.0 wt % IRP69-Ag 36

75 grams of IRP69-Ag (500 nm average particle size) was provided to LTL Color Compounders, Inc. (Morrisville, PA) and the material compounded into medical grade polypropylene. The compounding effort yielded 4 lbs. of modified PP and 20 molded coupons of a light tan coloration. Examination of the surface revealed excellent characteristics and antimicrobial testing by ASTM E2180 demonstrated excellent effectiveness against Pseudomonas aeruginosa. In addition to the above-mentioned fabrication constraints, we have observed improved stability of the attached cations based on thermal gravimetric analysis work.

For example, benzalkonium chloride (melting point=35° C.) is an inhibitor of cure in 2-part platinum curing silicones. In the event that the simple salt does not inhibit cure, it would be molten during cure and thus leak or ooze from any curing (molded or extruded) material. Once bound to the organic ion exchange resin, the concern over melting is eliminated because of the bulk nature of the crosslinked organic ion exchange copolymers. Further, to our surprise, cure is not inhibited when incorporated into 2-part platinum cured silicones.

Measurement of the thermal stability of the benzalkonium salt of IRP69 (IRP69-benzalkonium) by thermal gravimetric analysis was carried out and compared it to the stability of benzalkonium chloride. The thermogravimetric profile demonstrates that benzalkonium chloride begins to decompose at about 200° C. whereas the IRP69-benzalkonium (BA-SCER) salt is very stable to a temperature approaching 300° C. (FIG. 6).

### Example 64

### Preparation of Strong Cation Exchange Resin (SCER) Trimethoprim Complex

Strong cation exchange resin is treated with diluted HCl (8%) either with the use of a column or by repetitive acidification in a glass chemical reactor to transform the sodium salt of the ion exchange resin into the acid form. The resin is rinsed free of free acid and the resin is filtered under vacuum and allowed to air dry. Trimethoprim (2,4-diamino-5-(3,4,5-trimethoxy-benzyl)-pyrimidine) is added to the acidified resin and the mixture allowed to stir. Trimethoprim is soluble at 0.4 mg/mL. The solution is then stirred for 2 hours and the resin then filtered. The resin is dried under vacuum and milled to 1-10 micron.

### Example 65

### Preparation of Strong Cation Exchange Resin (SCER) Sulfamethoxazole Complex

Strong cation exchange resin is treated with diluted HCl (8%) either with the use of a column or by repetitive acidification in a glass chemical reactor to transform the sodium salt of the ion exchange resin into the acid form. The resin is rinsed free of free acid and the resin then filtered under vacuum and allowed to air dry. Sulfamethoxazole (4-amino-N-(5-methyl-1,2-oxazol-3-yl)benzenesulfonamide) is added to the acidified resin and the mixture allowed to stir. Sulfamethoxazole is soluble at <1.0 mg/mL. The solution is then stirred for 2 hours and the resin is filtered. The resin is dried under vacuum and milled to 1-10 micron.

### Example 66

### Preparation of Strong Cation Exchange Resin (SCER) Dapsone Complex

Strong cation exchange resin is treated with diluted HCl (8%) either with the use of a column or by repetitive acidification in a glass chemical reactor to transform the sodium salt of the ion exchange resin into the acid form. The resin is rinsed free of free acid and the resin then filtered under vacuum and allowed to air dry. Dapsone (diaminodiphenylsulfone (DDS)) is added to the acidified resin and the mixture allowed to stir. diaminodiphenylsulfone (DDS) is soluble at <1.0 mg/mL. The solution is then stirred for 2 hours and the resin is filtered. The resin is dried under vacuum and milled to 1-10 micron.

### Example 67

### Antibacterial, Antifungal and Antiviral Testing of Various SCER-Modified Decorative Paint Composites

The following IMS Strong Cation Exchange Resins were evaluated: Benzalkonium (BA-SCER), Benzethonium (BZT-SCER), Copper (II) Cu-SCER, Cetylpyridinium (CP-SCER), BA/Cu-SCER and Zinc (Zn-SCER) activity was evaluated at various % active loading (0.75-5.00%) in a PPG (Glidden) Interior Acrylic Latex Enamel Paint (Glidden Premium Semigloss Paint plus Primer). The methods for testing included ASTM E2180-18 Standard Test Method for Determining the Activity of Incorporated Antimicrobial Agent(s) in Polymeric or Hydrophobic Materials and D3273-16 Standard Test Method of Resistance to Growth of Mold on the Surface of Interior Coatings in an Environmental Chamber (Modified). The following bacterial log 10 knockdowns were achieved following 24-hours of exposure of an inoculum to the material surface. The Table below details ASTM E2180 Log 10 reductions for 2.00-5.00% BA-SCER, 3.00-5.00% BZT-SCER, 0.75-4.00% Cu-SCER, 2.00% CP-SCER, 2.00% Zn-SCER and 0.50-1.00% wt. % BA/Cu-SCER-modified Semi-gloss Glidden Interior Latex Paint.

TABLE
ASTM E2180. Log10 bacterial reductions from paint surfaces modified with IMS actives

| IASIS | % Active | E. | A. | K. | | P. | S. | E. | S. |
|---|---|---|---|---|---|---|---|---|---|
| Active | Load | coli | baumanii | pneumoniae | MRSA | aeruginosa | aureus | faecalis | epidermidis |
| BA-SCER | 2.00% | | | | 4.85 | | 5.80 | | |
| BA-SCER | 3.00% | | | | | | 5.26 | | |
| BA-SCER | 5.00% | | | | | | 5.26 | | |
| BZT-SCER* | 3.00% | | | 4.69 | | | | | |
| BZT-SCER* | 5.00% | | | 5.26 | | | | | |
| Cu-SCER | 0.75% | 5.80 | | 5.80 | | 3.70 | 5.80 | | 5.80 |
| Cu-SCER | 1.00% | | | 4.91 | 4.85 | | | | |
| Cu-SCER | 1.25% | | | 5.92 | 4.85 | | | | |
| Cu-SCER | 1.50% | 5.80 | 4.30 | 5.80 | 4.68 | 5.80 | 5.80 | 5.80 | 5.80 |
| Cu-SCER | 4.00% | 5.40 | | | | | 5.60 | | |
| CP-SCER | 2.00% | 5.00 | | | | | 5.88 | | |
| BA-SCER & | 0.50% | | | | | | 4.68 | | |
| Cu-SCER | 0.50% | | | | | | | | |
| BA-SCER & | 0.75% | | | | 3.63 | 4.19 | | | |
| Cu-SCER | 0.75% | | | | | | | | |
| BA-SCER & | 1.00% | | | 6.09 | 5.57 | 4.56 | 5.11 | | |
| Cu-SCER | 1.00% | | | | | | | | |
| Zn-SCER | 2.00% | 5.00 | | | | | 5.88 | | |

The foregoing data demonstrate significant bacterial log 10 reduction in the paint containing 1.5% SCER-Cu. Eight bacterial microorganisms were evaluated in a 24-hour format by ASTM E2180. The 1.5% SCER-Cu active reduced bacterial pathogens by >4.30 logs. Five additional paints (BA-SCER, BZT-SCER, CP-SCER, BA and Cu-SCER and Zn-SCER) were evaluated against multiple organisms. All paints effectively reduced bacterial pathogens at a minimum of ≥3.63 logs.

Additional studies employed the ASTM D3273-16 Standard Test Method of Resistance to Growth of Mold on the Surface of Interior Coatings in an Environmental Chamber (Modified) utilizing Stachybotrys chartarum as the test organism against Leneta scrub test panels painted with PPG Glidden Premium Semigloss Paint+Primer modified to include IMS 1.5% Cu-SCER. Painted surfaces treated with Cu-SCER at 1.5% load resulted in a surface that inhibited the growth of Stachybotrys chartarum under ideal growth conditions over 4 weeks. The fungal resistance testing demonstrated that under the conditions of treatment, the 1.5 wt. % Cu-SCER loaded paint resulted in a surface that inhibited the growth of Stachybotrys chartarum under the ideal growth conditions that the test article was subjected to over a period of 4-weeks.

TABLE
Antifungal Testing of a latex paint with Cu-SCER additive.

| | % | | | |
|---|---|---|---|---|
| IASIS | Active | Laboratory | | |
| Active | load | Method | Organism | Result |
| Cu- | 1.50% | Nova Biologicals | Stachybotrys | No Growth Observed |
| SCER | | Conroe, TX 77301 | chartarum | on 3 Replicates after 4 |
| | | ASTM D3273-16 | | weeks at 37° C./High |
| | | | | Humidity; All controls |
| | | | | behaved as expected. |

Additional studies employed ISO 21702:2019 Evaluation of the Virucidal Properties of a Treated Non-Porous Material Versus Coronavirus. The virucidal effects of the test surface was compared to the untreated control. Coronavirus strain 229E (ATCC #VR-740) as the test virus against Leneta scrub test panels painted with PPG Glidden Premium Semigloss Paint+Primer modified to include IMS 2.00% Cu-SCER. Painted surfaces treated with Cu-SCER at 2.00% load resulted in a surface that reduced coronavirus 99.68% in 2 hours and 99.92% in 24 hours. Virucidal activity, as exemplified here for compositions and methods of the invention, against the standard Coronavirus strain 229E are widely accepted as predictive against all known human Coronaviruses, including Sudden Acute Respiratory Syndrome (SARS) Coronaviruses (such as SARS-COV-1 and SARS-COV-2 (COVID-19) Coronaviruses).

TABLE
Antiviral testing of a latex paint with Cu-SCER additive.

| | % | | | 2 Hour | 24 Hour |
|---|---|---|---|---|---|
| IASIS | Active | | | Reduction | Reduction |
| Active | load | Laboratory; Method | Organism | % | % |
| Cu-SCER | 2.00% | Bioscience | Coronavirus | 99.68 | 99.92 |
| | | Laboratories Inc.; | strain 229E | | |
| | | Bozeman, MT 59718; | (ATCC#VR-740) | | |
| | | ISO 21702 | | | |

Additional studies employed EPA-#01-1A is the Protocol for Residual Self-Sanitizing Activity of Dried Chemical Residues on Hard, Non-Porous Surfaces used to determine the residual sanitizing efficacy of antimicrobial products after application to inanimate, nonporous, non-food contact hard surfaces. MRSA and P. aeruginosa were evaluated as the test organisms against Leneta scrub test panels painted with PPG Glidden Premium Semi-gloss Paint+IMS Primer modified to include IMS 1.50 and 2.00% Cu-SCER. Positive Controls were prepared using Sherwin Williams Paint Shield®. The IMS modified paints performed as well or better than the Sherwin Williams Paint Shield® control for both organisms as seen in table below.

TABLE
EPA#01-1A.
Log10 bacterial reductions from paint formulations modified to include
with IASIS additives vs. Sherwin Williams Paint Shield ®.

| IASIS Active | % Active load | P. aeruginosa | MRSA |
|---|---|---|---|
| Cu-SCER | 1.50 | 4.77 | |
| Cu-SCER | 2.00 | | 3.88 |
| Sherwin Williams Paint | | 3.08 | 3.64 |
| Shield ® (SWPS) | | | |

Additional studies employed extraction of paint samples with an industrial Lysol formulation and tap water. Leneta scrub test panels painted with PPG Glidden Premium Semigloss Paint+Primer modified to include IMS 1.50% Cu-SCER is effective against eight tested organisms. Extraction of these samples in Lysol® and tap water at room temperature for a week, does not reduce efficacy. The extracted samples were evaluated against K. pneumoniae and MRSA. The slightly lower results for MRSA reduction reflect a total kill with a slower growth rate in the Time “0” control.

TABLE
Extraction of IASIS Actives in Painted Samples. Log10 Reductions

| IASIS | | | | |
|---|---|---|---|---|
| Active | % Active Load | Extracted In | K. pneumoniae | MRSA |
| Cu-SCER | 1.50% | Professional | 4.79 | 3.92 |
| | | Lysol ® | | |
| | | Brand | | |
| | | Cleaner | | |
| Cu-SCER | 1.50% | Tap Water | 4.79 | 3.92 |

### Example 68

### Antibacterial Testing of Benzalkonium, Copper and Silver-Modified SCER Epoxy Composites

Cu-SCER (Cu(II)), BA-SCER (Benzalkonium), BHT-SCER (Benzethonium) and Ag-SCER (Silver) were tested at various wt. % loadings in Max Clear Food Grade Epoxy. The method for testing was ASTM E2180-18 Standard Test Method for Determining the Activity of Incorporated Antimicrobial Agent(s) in Polymeric or Hydrophobic Materials.

Log 10 knockdowns that were achieved at 24-hours post exposure of inoculums to the modified epoxy surfaces are detailed in Table 4.

### Table: ASTM E2180 Log 10 Reductions: Max Clear Food Grade Epoxy Modified to Include Antimicrobial Strong Cation Exchange Resins.

Note: The calculated Log 10 reductions are dependent on the bacterial counts determined from controls at T₀.

*Not Determined

The foregoing data show significant log 10 reductions for all three of the modified epoxies at various wt. % loading (0.50-5.0%), as demonstrated for both E. coli, P. aeruginosa, P. Mirabilis, S. epidermidis and S. aureus. Comparable evaluations for additional microbes on other types of in-house industrial epoxies are ongoing.

Additionally, Cu(II)-SCER loaded at 4.00 wt. %, was extracted in bleach (3-8%) for 1 week at 37° C. and demonstrated a 3.79 log reduction for S. aureus and a 5.29 log reduction for E. coli.

TABLE
Log10 Bacterial Reductions: Max Clear Epoxies modified with AM-IE Resin Additives

| IASIS | % Active | E. | S. | S. | P. | Klebsiella | P. |
|---|---|---|---|---|---|---|---|
| Active | Load | coli | epidermis | aureus | aeruginosa | pneumonaie | Mirabills |
| Ag-SCER | 0.50% | | | 5.72 | | | 5.49 |
| Ag-SCER | 0.75% | | | 5.72 | | | |
| Ag-SCER | 1.00% | | | 4.76 | | | |
| Ag-SCER | 4.00% | | 5.06 | | 6.04 | | |
| BA-SCER | 3.00% | | | 4.32 | | | |
| BZT-SCER | 3.00% | | | 4.31 | | | |
| Cu (II)-SCER | 1.00% | 6.14 | | ND | | | |
| Cu (II)-SCER | 2.00% | 5.36 | | 5.18 | | | |
| Cu (II)-SCER | 4.00% | 5.36 | | 5.08 | | | |
| Cu (II)-SCER | 5.00% | | | | | 4.36 | |

### Example 69

### Silicone Composites; Antibacterial Testing

Silicone slabs containing Ag-SCER additive at 0.75 and 1.5 wt % loadings were fabricated at Bentec Medical (Woodland, CA) and subsequently coated with polyvinylpyrrolidone and the materials were evaluated at IMS. The evaluation method was the ASTM E2180-18 Standard Test Method for Determining the Activity of Incorporated Antimicrobial Agent(s) in Polymeric or Hydrophobic Materials. The results determined from these experiments are detailed in Table 5.

TABLE
Silicone with Ag-SCER (low/high %)

| IASIS | % Active | | | |
|---|---|---|---|---|
| Active | Load | E. coli | P. mirabilis | MRSA |
| Ag-SCER | 0.75% | 5.42 | 5.34 | 5.30 |
| Ag-SCER | 1.5% | 5.77 | 5.34 | 6.02 |

The foregoing data demonstrate significant log 10 reductions in colony counts following exposure of each of the three microorganisms to the Ag-SCER-modified silicones at both 0.75 and 1.5% loadings. For these three microorganisms (E. coli, P. mirabilis and S. aureus) each of two concentrations tested for the Ag-SCER composites reduced pathogens by ≥5.30 logs.

### Example 70

### (Polyvinylchloride (PVC) Composites; Antibacterial Testing by ASTM E2180

Flexible and rigid PVC slabs were formulated for IMS by a domestic custom compounder. Ag-SCER additive concentrations of 0.75 wt. %, 1.00%, 1.25% and 1.5 wt. % were subsequently evaluated. Cu-SCER in Flexible PVC at 1.25% and 1.50 wt. % and in Rigid PVC at 2.00 wt. % PVC was evaluated in the same manner. BA-SCER in PVC at 1.50% was also evaluated. The testing method utilized was the ASTM E2180-18 Standard Test Method for Determining the Activity of Incorporated Antimicrobial Agent(s) in Polymeric or Hydrophobic Materials. Results from the assay are summarized in Table below.

TABLE
Log10 Reductions of Bacteria by Antimicrobial Resins incorporated into PVC

| IASIS | % Active | A. | E. | K. | | P. | P. |
|---|---|---|---|---|---|---|---|
| Active | Load | baumanii | coli | pneumoniae | MRSA | aeruginosa | mirabilis |
| Ag-SCER | 0.75 | 5.70 | 5.55 | 6.13 | 5.71 | 7.18 | |
| Ag-SCER | 1.00 | 5.70 | 5.55 | 6.13 | 5.71 | 7.18 | |
| Ag-SCER | 1.25 | 5.70 | 5.55 | 5.87 | 5.13 | 7.05 | |
| Ag-SCER | 1.50 | 5.70 | 5.55 | 6.13 | 5.13 | 7.05 | |
| Cu-SCER | 1.25 | | 4.97 | | 4.75 | | 4.80 |
| Cu-SCER | 1.50 | | 4.63 | | 4.00 | | |
| Ba-SCER | 1.50 | | 4.63 | | 4.00 | | |
| Cu-SCER | 2.00 | | | 4.47 | 4.79 | | |
| | (Rigid | | | | | | |
| | PVC) | | | | | | |

Additional studies employed extraction of the composite using an accelerate aging method which demonstrated that PVC containing IMS active Ag-SCER at 1.25 wt. % remained antimicrobial throughout the duration of the study. All samples were able to reduce MRSA and P. aeruginosa ≥4.73 logs. All effluents were checked for silver content. No detectable level of silver was shown after 9 weeks in extraction.

### (Polyvinylchloride (PVC) Composites Extracted in Tap Water; Antibacterial Testing; ASTM E2180

Additional studies employed extraction of the composite using an accelerate aging method out to 19 weeks. In this study PVC (1.25% wt. Ag-SCER) was extracted in tap water at 42° C. for 19 weeks. The material remained active for the length of the study (19 weeks) or 2.9 years of accelerated lifetime. See Table below. Effluents were tested for silver content throughout the study. No detectable silver was measured.

### Bacterial Log Reductions in PVC Samples Extracted in Water

| | | Weeks Extracted | | |
|---|---|---|---|---|
| | | at 42º C. | | |
| | % Active | (8x acceleration | | Log |
| IASIS Active | Load | factor, Arrhenius) | Organism | Reduction |
| Ag-SCER | 1.25 | 3 | MRSA | 5.31 |
| Ag-SCER | 1.25 | 3 | P. aeruginosa | 5.91 |
| Ag-SCER | 1.25 | 6 | MRSA | 4.73 |
| Ag-SCER | 1.25 | 6 | P. aeruginosa | 6.82 |
| Ag-SCER | 1.25 | 9 | MRSA | 6.11 |
| Ag-SCER | 1.25 | 9 | P. aeruginosa | 5.83 |
| Ag-SCER | 1.25 | 19 | MRSA | 5.32 |
| Ag-SCER | 1.25 | 19 | P. aeruginosa | 6.87 |

Additional studies employed extraction of the composite using an accelerate aging method out to 5 days. Flexible PVC containing Ag-SCER at 1.5 wt. % was evaluated in an extraction experiment using Artificial Urine (AU). The PVC was effective at killing E. coli and S. aureus at a log reduction of ≥4.34

TABLE
Bacterial Reduction of PVC with 1.5 wt. % Ag-SCER in
AU after 5-days of extraction.

| | % Active | | |
|---|---|---|---|
| IASIS Active | Load | E. coli | S. aureus |
| Ag-SCER | 1.5 | 4.34 | 4.97 |

The foregoing data demonstrate significant log 10 reductions observed for PVCs modified with Ag-SCER at both 0.75 and 1.5 wt % concentrations. Four microorganisms were utilized in the evaluations. Both concentrations of Ag-SCER in PVC reduced the identified bacterial pathogens by ≥5.55 logs (Table 4a). Significant (≥4.00) log 10 reductions in E. coli and MRSA were noted following exposure of inoculums to the PVC containing 1.5% Cu-SCER.

### Example 71

### Antimicrobial Surface Viability Following Extraction with Artificial Urine

Q7-4750 silicone was modified to include 1.50 wt % Ag-SCER and slabs were injection molded. The slab was cut into 1×1 in. test articles and the material extracted in artificial urine (AU) for 28-days (4-weeks) with weekly changes of AU. At each 1-week time point, a (9) samples were removed from test and 3-sets of (3) samples evaluated against each of Escherichia coli, methicillin-resistant Staphylococcus aureus, and Proteus mirabilis using the ASTM E2180 Standard Test Method for Determining the Activity of Incorporated Antimicrobial Agent(s) in Polymeric or Hydrophobic Materials. At each of the time points, the materials yielded Log 10 reductions that were in excess of 5.0. At day 28 the resulting reductions were >5.5 Logs. The results are shown in the following Example.

### Example 72

### Time-to-Kill (TTK) Reductions of Escherichia coli vs. a Silicone-Ag-SCER Composite

The silicone composite described in Example 71 was exposed to a broth containing 108 CFUs of Escherichia coli in artificial urine and the samples were allowed to incubate for 3-hours at 37° C. Samples were removed, lightly rinsed to remove non-adherent bacteria and total counts following exposure determined by removing bacteria from the surfaces (sonication and vortexing) and the samples placed into artificial urine and samples incubated at 37° C. Samples were removed at 3, 8, 16, 24, and 32-hours and counts determined using the same methodology. The Log 10 reduction results are shown in the following Table.

Ag-SCER silicone was assessed in suspension tests for time-kill efficacy over a 32-hour period. Escherichia coli and Methicillin-resistant Staphylococcus Aureus were challenged with 1.5% Ag-SCER doped Q7-4750 (Dow Corning) silicone with a PVP (Hydromer) coating in artificial urine (AU) for 32 hours, with samples screened in triplicate at 3 hours, 8 hours, 16 hours, 24 hours, and 32 hours and compared to Q7-4750 control silicone with no coating. One set of samples was used for plate count enumeration, and one set of samples was used for fluorescent imaging. Coated 1.5% Ag-SCER Ag samples reduced E. coli by >4 log 10 within 16 hours and left no CFU for enumeration within 24-hours (Table below) and reduced MRSA by >3 log 10 within 16 hours and left no CFU for enumeration within 32 hours. The data is represented in the following table and graphically in FIG. 7 . . . . Results for E. coli were confirmed using a live/dead cell fluorescence assay over the time course of the experiment.

TABLE
Average CFU/cm² and Average Log10 Reduction of E. coli by
1.5% Ag-SCER Samples

| Sample No | Hours exposed | CFU/cm² | Log10 Red. |
|---|---|---|---|
| T3 Ag | 3 | 488.1 | 2.44 |
| T8 Ag | 8 | 240.5 | 3.06 |
| T16 Ag | 16 | 187.4 | 4.72 |
| T24 Ag | 24 | 10.61 | 5.95 |
| T32 Ag | 32 | 10.61 | 5.98 |

### Example 73

### Inoculation of 1.5 wt % Ag-SCER-Modified Tubing in a Flow-Loop System Designed to Mimic an Artificial Bladder

Extruded silicone composite tubing (1.5 wt % Ag-SCER) was placed in line in a flow system with a 300 mL reservoir maintained at 37° C. Following a 1-hour conditioning at temperature, the reservoir was inoculated with 10e5 CFUs/mL of Escherichia coli 67 under flow. The graphical representation in FIG. 8 details the time-to-kill revealing demonstrates that all bacteria were killed within 4-hours. In similar fashion, the test system was inoculated with 10e7 CFUs of Escherichia coli the time-to-kill (TTK) increases. The data reveal (FIG. 9) that TTK is >8 hrs, and ≤24-hrs.

### Example 74

### Formation of a Novel Silicone Composite Foley Catheter Retention Balloon

Nusil Technologies Med-6440 (parts A & B) were combined with Ag-SCER to yield a final composition of 2.5 wt % Ag-SCER content. The mixture was dip coated onto a metal rod supported PTFE spheroid and the solvent allowed to evaporate and another coat subsequently added. Once dry the film was placed into a 150° C. oven for 10-minutes. The film (balloon) was subsequently stripped from the ball and inflated to 2.5× the volume using tap water. The balloon was tied off and allowed to sit until the water had evaporated through the thin film. The balloon was reinflated, and the process repeated. The balloon showed no signs of failure or leakage after several weeks of inflation.

### Example 75

### 4-week and 26-Week Implant Study

ISO 10993 4-Wk. implant study-microscopically the test articles caused minimal or no reaction in the tissue as compared to the negative control article. The test articles were determined to be non-reactive.

ISO 10993 26-Wk. implant study macroscopic evaluations showed no visible reaction at any test or control site. Classification=no significant tissue contact irritation. Microscopically each test article caused a minimal or no reaction as compared to the negative control article.

### Example 76

### Lithium Strong Cation Exchange Resin Controlled Release Device for Delivery of Lithium Carbonate to Treat Mood Disorders

Sulfonated polystyrene divinylbenzene copolymer in its acid form was combined with lithium acetate in water and the reaction stirred until exchange was complete. The material was isolated and milled to a fine particulate of about 1-10 micron size distribution. The powder was subsequently incorporated into a silicone matrix at concentrations ranging from 5-75 wt %. In order to provide delivery closer to zero order the silicone was combined with a water soluble, biocompatible polymer such as polyvinylpyrrolidone prior to vulcanization. The devices were placed into saline solution and the release of Li⁺X⁻ monitored using inductively coupled plasma atomic emission spectroscopy (ICP AES).

### Example 77

### Solid State Silicone Composite Lithium Ion Battery Separator

Sulfonated polystyrene divinylbenzene copolymer in its acid form was combined with lithium acetate in water and the reaction stirred until exchange was complete. The material was isolated and milled to a fine particulate of ≤0.4 micron average particle size in the distribution. The powder was subsequently incorporated into a liquid silicone elastomer matrix at concentrations ranging from 40-80 wt % loading and the mixture cast onto PTFE as a 25-micron film using an automatic film coater (doctor blade). The films were subsequently cured at 150° C. for 10-minutes and the films removed from the release liner for electrochemical impedance spectroscopy (EIS) measurement.

### Example 78

### Solid State Polyethylene Composite Lithium Ion Battery Separator

Sulfonated polystyrene divinylbenzene copolymer in its acid form was combined with lithium acetate in water and the reaction stirred until exchange was complete. The material was isolated and milled to a fine particulate of ≤0.4 micron average particle size in the distribution. The electronically active powder was subsequently incorporated into polypropylene by mixing in the melt at concentrations ranging from 40-80 wt % loading and the mixture press melted into thin film (25 micron in a heated press) between PTFE sheets. The films were subsequently removed from the release liner for electrochemical impedance spectroscopy (EIS) measurement.

This process can be applied to any variety of thermoset polymers (epoxies, silicones, urethanes) or thermoplastic materials to include polyethylene, polyurethane, TPEs including styrene-copolymers such as styrene isobutylene, and styrene-ethylene-butylene-styrene copolymers.

### Example 79

### Self-Disinfecting Cu(II)-SCER and Fe(II)-SCER Epoxy Surfaces with Generated Hydroxyl Radical Demonstrated by Methylene Blue (MB) Bleaching

MAX CLEAR epoxy resin (Food Safe, FDA Compliant) was formulated to include 4.0 wt % Cu(II)-SCER (1-10 micron) and the material was roll-coated onto a Leneta scrub test panel. The formulation was allowed to cure at room temperature for 4 hours and placed into a 50° C. oven for 12 hours. A MB bleaching test was adapted from Satoh, A. Y., Trosko, J. E., Masten, S. J., Methylene Blue Test for Rapid Qualitative Detection of Hydroxyl Radicals Formed In a Fenton's Reaction Aqueous Solution, Environ. Sci. Technol. 2007, 41, 2881-2887.

A 1.0 mmol MB dye solution was generated in deionized and deoxygenated water. A pipette was used to dispense approximately 0.25 mL of MB solution onto the Cu-SCER modified epoxy surface and a few drops of 3% hydrogen peroxide (stabilized) solution added to the solution of the dye. No change was observed over a period of 2-minutes. Another several drops of the hydrogen peroxide were added to the pooled liquid and an equal volume of saline added to the pool. Within a matter of seconds bleaching was evident and within 2-minutes the characteristic MB color was absent. The same experiment was conducted with a 4.0 wt % Fe(II)-SCER modified MAX CLEAR formulation. An identical outcome to the Cu(II)-SCER-modified MAX CLEAR epoxy surface was observed.

### Example 80

### Self-Disinfecting Cu(II)-SCER and Fe(II)-SCER Polyvinylchloride (PVC) Surfaces with Generated Hydroxyl Radical Demonstrated by Methylene Blue (MB) Bleaching

PVC was softened on a 2-roll mill and Cu(II)-SCER incorporated at 0.5, 0.75, 1.0, 1.25 and 1.50 wt % loading and the resulting formulations fashioned into plaques. A 1.0 mmol MB dye solution was generated in deionized and deoxygenated water. A pipette was used to dispense approximately 0.25 mL of MB solution onto the Cu-SCER modified PVC surface and a few drops of 3% hydrogen peroxide (stabilized) solution added to the solution of the dye. No change was observed over a period of 2-minutes. Another several drops of the hydrogen peroxide were added to the pooled liquid and an equal volume of saline added to the pool. Within a matter of seconds bleaching was evident and within 2-minutes the characteristic MB color was absent.

### Example 81

### Preparation of Cysteamine Salts of Strong Cation Exchange Resins

Sulfonated polystyrene divinylbenzene copolymer in its acid form (H⁺-SCER) was combined with cysteamine at an exchange capacity determined by the titration of the acid form of the SCER. Cysteamine is an approved drug in its salt form for the treatment of cystinosis, the abnormal accumulation of cystine which leads to crystallization in different tissue regions. Cysteamine is also a potent inhibitor of urease, an enzyme produced by certain bacteria (e.g. P. mirabilis) resulting in increased pH and the precipitation of mineral encrusted biofilms in urinary catheterized patients. Following stirring for 2-hours the resin was filtered, washed with warm DI water and the resin dried under vacuum for 18 hours at 85° C. The resulting resin was weighed, and the mass balance indicated 100% incorporation onto the resin backbone. The resin was milled to 1-10 micron particle size using a dry process and a zirconia-lined jar and media. The material was stable as it showed no signs of discoloration or odor. The material was incorporated into an LSR silicone at 2.0 wt %. Curing at 150° C. for 10 minutes caused no undue harm.

### Example 82

### Preparation of Chitosan Salts of Strong Cation Exchange Resins

Sulfonated polystyrene divinylbenzene copolymer in its acid form (HT-SCER) was combined with chitosan (base) at an exchange capacity determined by the titration of the acid form of the SCER. Chitosan dissolves (leaves solution) as it binds to the polymer via protonation to form the Chitosan-SCER complex. Chitosan is a known hemostatic agent. Following stirring for 2-hours the resin was filtered, washed with warm DI water and the resin dried under vacuum for 18 hours at 85° C. The resulting resin was weighed, and the mass balance indicated significant incorporation of chitosan onto the resin backbone. The resin was milled to 1-10 micron particle size using a dry process and a zirconia-lined jar and media. The material was stable as it showed no signs of discoloration. Subsequently, the resin was incorporated into a Baymedix FP505 foam as a surrogate for a hemostatic dressing.

### Example 83

### Preparation of Lithium Strong Cation Exchange Resin Modified Polypropylene Thin Films

Li-SCER (0.4 micron particle size) was combined with polypropylene in the melt at 50 wt % loading. The resulting slurry was pelletized and subsequently injection molded into a 3×3 in plaque. A 1×1 cm segment of the plaque was placed onto PTFE sheet and a 25-micron thick stainless frame (6×6 in) placed around the plaque and another PTFE sheet placed over the top. The configuration was placed into a heated press set to 180° C. and pressure applied to the level of the SS frame for 45 seconds. The pressure was released, and the entire configuration removed from the press and allowed to cool. The resulting film possessed good flexibility.

### Example 84

### (Polyethylene) Composites; Antibacterial Testing; ASTM E2180

Polyethylene slabs were formulated for IMS by a domestic custom compounder. Ag-SCER additive concentrations of 1.0 wt. % and 2.0 wt. % were evaluated by ASTM E2180-18; Standard Test Method for Determining the Activity of Incorporated Antimicrobial Agent(s) in Polymeric or Hydrophobic Materials. Results from the assay are summarized in Table below. Both 1.00 and 2.00% wt. Ag-SCER polyethylene test articles reduced S. aureus, E. coli and P. aeruginosa by ≥4.3 LR logs.

TABLE
Log10 Reductions of Bacteria by Antimicrobial Resins incorporated
into Polyethylene

| | % | | | |
|---|---|---|---|---|
| IASIS | Active | Staphylococcus | Escherichia | Pseudomonas |
| Active | Load | aureus | coli | aeruginosa |
| Ag-SCER | 1.00% | ≥4.4 LR | | |
| Ag-SCER | 2.00% | ≥4.4 LR | | |
| Ag-SCER | 1.00% | | ≥4.3 LR | |
| Ag-SCER | 2.00% | | ≥4.3 LR | |
| Ag-SCER | 1.00% | | | ≥4.9 LR |
| Ag-SCER | 2.00% | | | ≥4.9 LR |
| Ag-SCER | 1.00% | ≥4.4 LR | ≥4.4 LR | ≥4.7 LR |

### Example 85

### Silicone Composites; Antibacterial Testing; ASTM E2180

Test articles were prepared using either NuSil MED 4950 or Dow Q7-4750. A series of samples were created using IMS Active Ag-SCER at various wt. % loads (0.25-2.00%). Silicone samples were created by weighing out “Part A” of either the NuSil or Dow brand. To Part A, the Active was added and mixed with a speed mixer for a few minutes. “Part B” was then weighed out and directly into the speed mixer cup containing “Part A” plus IMS active. The cup was mixed thoroughly and transferred to a syringe. The mixture was dispensed and leveled onto a stainless-steel mold and cured at 150° C. for ≥30 minutes. Cure was not inhibited. Samples were evaluated per ASTM E2180.

TABLE
Ag-SCER + NuSil MED4950 and Dow Q7-4750 Silicone Log
Reductions

| | % Active | | | | |
|---|---|---|---|---|---|
| Active/Silicone | Load | E. coli | P. mirabilis | MRSA | S. aureus |
| Ag-SCER | 0.25% | 5.63 | | | 5.34 |
| (NuSil) | | | | | |
| Ag-SCER | 0.50% | 5.87 | | | 5.30 |
| (NuSil) | | | | | |
| Ag-SCER | 0.75% | 5.34 | 5.34 | 5.42 | |
| (Dow) | | | | | |
| Ag-SCER | 1.00% | 6.05 | | | 5.46 |
| (NuSil) | | | | | |
| Ag-SCER | 1.25% | 5.78 | | 5.42 | |
| Ag-SCER | 1.50% | 5.34 | 5.34 | 5.77 | |
| (Dow) | | | | | |
| Ag-SCER | 2.00% | | 4.48 | 4.59 | 5.26 |
| (NuSil) | | | | | |

TABLE
BA-SCER, Cetyl Pyridinium-SCER + NuSil MED 4950 Silicone
Log Reductions

| | % Active | | | |
|---|---|---|---|---|
| IASIS Active | Load | E. coli | S. aureus | MRSA |
| Ba-SCER | 1.50% | | 5.08 | |
| CP-SCER | 1.50% | | 5.28 | |

### Example 86

### Silicone Composites; Antibacterial Testing of a Wound Contact Layer; ASTM E2180

Thin rolls of Ag-SCER Dow-4750, extruded by Bentec (x mm thick) were evaluated against the following organisms listed in table below. A subsequent set of samples were extracted in hydrogen peroxide prior to testing A noted color change was observed in samples with exposure to H₂O₂. The color change noted in the thin silicone went from a light caramel to a creamy white after exposure to H₂O₂. All test articles were effective against an array of bacteria (>4.10 LR).

TABLE
0.75% Ag-SCER Dow Q7-4750 Silicone Log10 Reductions

| IASIS | % Active | | | | |
|---|---|---|---|---|---|
| Active | Load | MRSA | S. aureus | P. aeruginosa | E. coli |
| Ag-SCER | 0.75% | 4.10 | | 5.10 | |
| Ag-SCER + | 0.75% | | 4.78 | | 5.43 |
| H2O2 | | | | | |

### Example 87

### Silicone Composites; Artificial Urine Extraction; Antimicrobial Evaluation; ASTM E2180

The following tables summarize the AU extraction experiments for silicone made with 0.75-1.5 wt. % Ag-SCER. Both the 1.25 and 1.5 wt. % Ag-SCER silicones remained effective throughout the 28 day extraction, achieving bacterial log knockdowns of ≥5.54. The 0.75 and 1.00% wt. Ag-SCER silicones were effective for 2 and 3 weeks, respectively. Testing for both of these silicones was halted after week 3 data was observed.

TABLE
1.5% Ag-SCER Dow Q7-4750 Silicone Artificial Urine Extraction
Log10 Reductions

| | Day | | | | |
|---|---|---|---|---|---|
| | 0 | 7 | 14 | 21 | 28 |
| MRSA | 5.77 | 5.74 | 5.94 | 6.04 | 5.83 |
| E. coli | 5.34 | 5.99 | 5.99 | 5.28 | 5.60 |
| P. mirabilis | 5.34 | 5.99 | 5.99 | 6.04 | 5.54 |

TABLE
1.25% Ag-SCER MED4950 Liquid Silicone Rubber Artificial Urine
Extraction Log10 Reductions

| | Day | | | | |
|---|---|---|---|---|---|
| | 0 | 7 | 14 | 21 | 28 |
| MRSA | 5.42 | 5.65 | 5.65 | 5.65 | 5.65 |
| E. coli | 5.78 | 4.07 | 4.07 | 5.96 | 5.96 |

TABLE
1% Ag-SCER Dow Q7-4750 Silicone Artificial Urine Extraction
Log10 Reductions

| | Day | | | | |
|---|---|---|---|---|---|
| | 0 | 7 | 14 | 21 | 28 |
| MRSA | 4.97 | 5.47 | 5.47 | 1.42 | |
| E. coli | 5.42 | 6.09 | 5.51 | 2.04 | |

TABLE
0.75% Ag-SCER Dow Q7-4750 Silicone Artificial Urine Extraction
Log10 Reductions

| | Day | | | | |
|---|---|---|---|---|---|
| | 0 | 7 | 14 | 21 | 28 |
| MRSA | 5.42 | 5.77 | 3.32 | 3.63 | |
| E. coli | 5.34 | 5.98 | 3.75 | 2.13 | |
| P. mirabilis | 5.34 | 6.22 | 3.75 | 2.13 | |

### Example 88

### Alternative Milling Methods

Within certain aspects of the invention higher throughput hammer milling methods may be employed in the first milling step in order to generate particles with sizes of 1-10 microns, as demonstrated using a Hosakowa hammer mill. Similarly, jet milling may be of value in this first step, and cryogenic jet milling can produce finer particles and smaller sizes. Standard jet milling can readily produce particles in the 70-100 micron range. These sizes may be excellent for use in paints. Both hammer milling and jet milling will alleviate the need to use non-solvent milling techniques in all steps in order to provide particles of the optimal size. It is also worth noting that nanoparticulate materials are likely not needed for all applications. In fact, for building materials the incorporation of particles of approximately 10 microns will be acceptable in most cases. Where finer particulates are required, i.e. medical devices and sex toys for example a secondary reduction using planetary milling will be required. However, planetary milling may be vertical or horizontal in equipment much larger than our current jar-based batch method and the process can yield reproducible distributions in the single digit micron range using a dry milling method and stainless or ceramic grinding media.

### LIST OF REFERENCES CITED

Cozad, A., and R. D. Jones. 2003. Disinfection and the Prevention of Infectious Disease. Am. J. Infect. Control 31:243-254.
Aitken, C., and D. J. Jeffries. 2001. Nosocomial spread of viral disease. Clin. Microbiol. Rev. 14:528-546., Barker, J., D. Stevens, and S. F. Bloomfield. 2001
Barker, J., D. Stevens, and S. F. Bloomfield. 2001. Spread and prevention of some common viral infections in community facilities and domestic homes. J. Appl. Microbiol. 91:7-21.
England, B. L. 1982. Detection of viruses on fomites, p. 179-220. In C. P. Gerba and S. M. Goyal (ed.), Methods in environmental virology. Marcel Dekker, Inc., New York, N.Y.;
Haas, C. N., J. B. Rose, and C. P. Gerba. 1999, In Microbial agents and their transmission, p. 35-50. In C. N. Haas, J. B. Rose, and C. P. Gerba (ed.), Quantitative microbial risk assessment. J. Wiley and Sons, Inc., New York, N.Y.;
Reynolds, K. A., P. Watts, S. A. Boone, and C. P. Gerba. 2005. Occurrence of bacteria and biochemical biomarkers on public surfaces. Int. J. Environ. Health Res. 15:225-234;
Sattar, S. A. 2001. Survival of microorganisms on animate and inanimate surfaces and their disinfection, p. 195-205. In W. A. Rutala (ed.), Disinfection, sterilization and antisepsis: principles and practices in healthcare facilities. Association for Professionals in Infection Control and Epidemiology, Inc., Washington, D.C.)
Goldmann, D. A. 2000. Transmission of viral respiratory infections in the home. Pediatr. Infect. Dis. J. 19 (Suppl. 10): S97-S102.)
Standard Test Method for Determining the Activity of Incorporated Antimicrobial Agent(s) In Polymeric or Hydrophobic Materials, ASTM International, West Conshohocken, PA, 2007, Developed by Subcommittee: E35.15, Book of Standards Volume: 11.05, DOI: 10.1520/E2180-07R12, www.astm.org/

We claim:

1. An antiviral polymer composite material comprising a fine particulate polymer salt ionically associated with an antiviral active copper Cu(II) ion, the polymer salt dispersed within a thermoset, thermoplastic, or photocuring polymer, or a polymer lacquer, or a complex polymer composition including an emulsion, wherein the polymer or polymer composition forms a solid upon cooling, curing, or drying, wherein the antiviral active copper Cu(II) ion remains active in the solid composite after hardening, drying or curing, yielding a solid composite that possesses potent antiviral surface activity.

2. (canceled)

3. (canceled)

4. The antiviral polymer composite material of claim 1, wherein the polymer or polymer composition includes a polysiloxane, polyalkylene, polyamide, epoxy, polycarbonate, polyester, latex, vinyl, acrylic, or polyurethane, or combination thereof.

5. The antiviral polymer composite material of claim 1, wherein the thermoset or thermoplastic or photocuring polymer is formed by a process of: A) solidifying liquid or semi-solid polymer precursors; B) casting liquid or semi-solid polymer precursors prior to solidifying the thermoset or thermoplastic or photocuring polymer; C) providing a polymer lacquer comprising a solvent that is evaporated from the polymer lacquer during hardening of the thermoset or thermoplastic or photocuring polymer, or a combination of the processes or A, B and/or C.

6. The antiviral polymer composite material of claim 1, further activated by exposing a surface of the solid composite to an ion-containing hydrogen peroxide mixture or solution, which peroxide mixture or solution reacts with the antiviral active ionic agent to yield a reactive oxygen species that exerts additional antiviral activity.

7. The antiviral polymer composite of claim 1, comprising a coating formed of an epoxy, a polymer lacquer, an acrylic latex enamel, a polyurethane, a polyaspartate, or a polyester, wherein said coating exhibits potent antiviral surface activity.

8. (canceled)

9. The antiviral polymer composite of claim 7, wherein the coating exhibits potent antiviral activity against one or more target viruses selected from enveloped and non-enveloped viruses, including veterinary and human coronaviruses, influenza viruses, polioviruses, herpes viruses, hepatitis viruses, noroviruses, and veterinary and human immunodeficiency viruses.

10. The antiviral polymer composite of claim 9, wherein the coating exhibits potent antiviral activity against human coronaviruses, wherein a surface coated with the polymer composite exhibits a 95% or greater reduction of viral contamination/load after 2 hours post-inoculation compared to a control, un-coated inoculated surface.

11. The antiviral polymer composite of claim 7, wherein the coating is applied to a medical device, medical or institutional facility surface, such as a wall, floor, support structure, furnishing or touch surface of a hospital, prison, long-term care facility, community center, or mass occupancy ships, airliners and vehicles, medical equipment unit, or medical textile to render the device, facility surface, equipment or textile resistant to fungal or viral contamination.

12. The antiviral composite of claim 1, integrated within or applied to a surface of a medical device.

13. The biologically active, stable antiviral polymer composite of claim 12, wherein the medical device is selected from catheters, ureteral stents, pacemaker leads, shunts, wound drains, fluid drains, endotracheal tubes, cannulas, gastronomy tubes, and tracheostomy tubes.

14. The biologically active, stable antiviral polymer composite material according to claim 1, incorporated within or coated upon a surface of a polymeric textile or fabric or incorporated onto a metallic textile or fabric.

15. The antiviral composite material according to claim 14, wherein the textile or fabric forms an antiviral barrier or filtration surface of a personal protective mask, an air filtration filter module, personal protective wear, patient protective drapes, sanitary wipes and towels, bedding and linens, furniture coverings, and other antiviral protective textile materials.

16. An antiviral medical device having a surface construction or coating comprising an antiviral polymer composite material comprising a fine particulate polymer salt ionically associated with an antiviral active copper Cu(II) ion, the polymer salt dispersed within a thermoset, thermoplastic, or photocuring polymer, or a polymer lacquer, or a complex polymer composition including an emulsion, wherein the polymer or polymer composition is applied to or incorporated within the medical device to form a solid upon cooling, curing, or drying, wherein the antiviral active ionic copper Cu(II) ion remains active in the solid composite after hardening, drying or curing upon or within the medical device surface, yielding a solid composite, or coating, at the surface of the medical device that exhibits potent antiviral surface activity.

17. The antiviral medical device of claim 16, wherein the antiviral polymer the polymer or polymer composition includes a polysiloxane, polyalkylene, polyamide, epoxy, polycarbonate, polyester, latex, vinyl, acrylic, or polyurethane, or combination thereof.

18. The antiviral medical device of claim 16, wherein the antiviral polymer composite material of claim 1, is further activated by exposing a surface of the solid composite to an ion-containing hydrogen peroxide mixture or solution, which peroxide mixture or solution reacts with the antiviral active copper Cu(II) ion to yield a reactive oxygen species that exerts additional antiviral activity.

19. The antiviral medical device of claim 16, wherein the antiviral polymer composite material integrated within or coated upon the medical device confers potent antiviral activity of the medical device surface against one or more target viruses selected from enveloped and non-enveloped viruses, including veterinary and human coronaviruses, influenza viruses, polioviruses, herpes viruses, hepatitis viruses, noroviruses, and veterinary and human immunodeficiency viruses.

20. The antiviral medical device of claim 16, wherein the antiviral polymer composite material integrated within or coated upon the medical device confers potent antiviral activity of the medical device surface against human coronaviruses, wherein the device surface constructed of or coated with the antiviral polymer composite exhibits a 95% or greater reduction of viral contamination/load after 2 hours post-inoculation compared to a control, un-coated inoculated device surface.

21. The antiviral medical device of claim 16, wherein the medical device is selected from catheters, ureteral stents, pacemaker leads, shunts, wound drains, fluid drains, endotracheal tubes, cannulas, gastronomy tubes, and tracheostomy tubes.

22. The antiviral medical device of claim 16, wherein the device comprises a medical textile or fabric having the antiviral polymer composite material incorporated within or coated upon a surface of the textile or fabric.

23. The antiviral medical device of claim 21, wherein the textile or fabric forms an antiviral barrier or filtration surface of a personal protective mask, an air filtration filter module, personal protective wear, patient protective drapes, sanitary wipes and towels, bedding and linens, furniture coverings, and other antiviral protective textile materials
